(19) United States
(12) Patent Application Publication
(10) Pub. No.: US 2019/0040413 A1
(43) Pub. Date: Feb. 7, 2019

(54) MAIZE EVENT DP-032218-9 AND METHODS FOR DETECTION THEREOF

(71) Applicants: PIONEER HI-BRED INTERNATIONAL, INC., Johnston, IA (US); E. I. DU PONT DE NEMOURS AND COMPANY, Wilmington, DE (US)

(72) Inventors: MARY BEATTY, EARLHAM, IA (US); KENT BRINK, JOHNSTON, IA (US); VIRGINIA CRANE, DES MOINES, IA (US); SCOTT DIEHN, WEST DES MOINES, IA (US); ALBERT LU, WEST DES MOINES, IA (US); GREGORY J. YOUNG, BURLINGAME, CA (US)

(73) Assignees: PIONEER HI-BRED INTERNATIONAL, INC., JOHNSTON, IA (US); E. I. DU PONT DE NEMOURS AND COMPANY, WILMINGTON, DE (US)

(21) Appl. No.: 16/167,630

(22) Filed: Oct. 23, 2018

Related U.S. Application Data
(63) Continuation of application No. 15/113,626, filed on Jul. 22, 2016, now abandoned.
(60) Provisional application No. 61/756,897, filed on Jan. 25, 2013.

(51) Int. Cl. C12N 15/82; A01H 5/10; C12Q 1/6895

(52) U.S. Cl. CPC C12N 15/8286; Y02A 40/162; C12Q 2600/13; A01H 5/10; C12Q 1/6895; C12N 15/8277; C12Q 2600/158

(57) ABSTRACT

The disclosure provides DNA compositions that relate to transgenic insect resistant maize plants. Also provided are assays for detecting the presence of the maize DP-032218-9 event based on the DNA sequence of the recombinant construct inserted into the maize genome and the DNA sequences flanking the insertion site. Kits and conditions useful in conducting the assays are provided.

### REFERENCE TO SEQUENCE LISTING SUBMITTED ELECTRONICALLY

A sequence listing having the file name “5649USPCT2_SeqList.txt” created on Mar. 31, 2014, and having a size of 92 kilobytes is filed in computer readable form concurrently with the specification. The sequence listing is part of the specification and is herein incorporated by reference in its entirety.

### FIELD

Embodiments of the present disclosure relate to the field of plant molecular biology, specifically embodiment of the disclosure relate to DNA constructs for conferring insect resistance to a plant. Embodiments of the disclosure more specifically relate to insect resistant corn plant event DP-032218-9 and to assays for detecting the presence of corn event DP-032218-9 in a sample and compositions thereof.

### BACKGROUND

Corn is an important crop and is a primary food source in many areas of the world. Damage caused by insect pests is a major factor in the loss of the world's corn crops, despite the use of protective measures such as chemical pesticides. In view of this, insect resistance, via heterologous genes, has been introduced into crops such as corn in order to control insect damage and to reduce the need for traditional chemical pesticides.

The expression of heterologous genes in plants is known to be influenced by their location in the plant genome and will influence the overall phenotype of the plant in diverse ways. For this reason, it is common to produce hundreds to thousands of different events and screen those events for a single event that has desired transgene expression levels, patterns, and agronomic performance sufficient for commercial purposes. An event that has desired levels or patterns of transgene expression can be used for introgressing the transgene into other genetic backgrounds by sexual outcrossing using conventional breeding methods. Progeny of such crosses maintain the transgene expression characteristics of the original transformant. This strategy is used to ensure reliable gene expression in a number of varieties that are well adapted to local growing conditions.

It would be advantageous to be able to detect the presence of a particular event in order to determine whether progeny of a sexual cross contains an event of interest. In addition, a method for detecting a particular event would be helpful for complying with regulations requiring the pre-market approval and labeling of foods derived from recombinant crop plants, or for use in environmental monitoring, monitoring traits in crops in the field, or monitoring products derived from a crop harvest, as well as for use in ensuring compliance of parties subject to regulatory or contractual terms.

Therefore, a reliable, accurate, method of detecting transgenic event DP-032218-9 is needed.

### SUMMARY

Embodiments of this disclosure relate to methods for producing and selecting an insect resistant monocot crop plant. More specifically, a DNA construct is provided that when expressed in plant cells and plants confers resistance to insects. According to one aspect of the disclosure, a DNA construct, capable of introduction into and replication in a host cell, is provided that when expressed in plant cells and plants confers insect resistance to the plant cells and plants. Maize event DP-032218-9 was produced by Agrobacterium-mediated transformation with plasmid PHP36676. This event contains a cry2A.127, cry1A.88, Vip3Aa20, and mo-pat gene cassettes, which confer resistance to certain lepidopteran and coleopteran pests, as well as tolerance to phosphinothricin.

Specifically, the first cassette contains the cry2A. 127 gene encoding the Cry2A.127 protein that has been functionally optimized using DNA shuffling techniques and based on genes derived from Bacillus thuringiensis subsp. kurstaki. The 634-residue protein produced by expression of the cry2A. 127 sequence is targeted to maize chloroplasts through the addition of a 54-amino acid chloroplast transit peptide (CTP) (U.S. Pat. No. 7,563,863 B2) as well as a 4-amino acid linker (Peptide Linker) resulting in a total length of 694 amino acids (approximately 77 kDa) for the precursor protein (the CTP sequence is cleaved upon insertion into the chloroplast), resulting in a mature protein of 644 amino acids in length with an approximate molecular weight of 72 kDa; (SEQ ID NO: 8). The expression of the cry2A. 127 gene and the CTP is controlled by the promoter from the Citrus Yellow Mosaic Virus (CYMV) (Huang and Hartung, 2001, Journal of General Virology 82: 2549-2558; Genbank accession NC_003382.1) along with the intron 1 region from maize alcohol dehydrogenase gene (Adhi Intron) (Dennis et al., 1984, Nucleic Acids Research 12: 3983-4000). Transcription of the cry2A. 127 gene cassette is terminated by the presence of the terminator from the ubiquitin 3 (UBQ3) gene of Arabidopsis thaliana (Callis et al., 1995, Genetics 139: 921-939). In addition, a genomic fragment corresponding to the 3′ untranslated region from a ribosomal protein gene (RPG 3′ UTR) of Arabidopsis thaliana (Salanoubat et al., 2000, Nature 408: 820-822; TAIR accession AT3G28500) is located between the cry2A. 127 and cry1A.88 cassettes in order to prevent any potential transcriptional interference with downstream cassettes. Transcriptional interference is defined as the transcriptional suppression of one gene on another when both are in close proximity (Shearwin, et al., 2005, Trends in Genetics 21: 339-345). The presence of a transcriptional terminator between two cassettes has been shown to reduce the occurrence of transcriptional interference (Greger et al., 1998, Nucleic Acids Research 26: 1294-1300); the placement of multiple terminators between cassettes is intended to prevent this effect.

The second cassette (cry1A.88 gene cassette) contains a second shuffled insect control gene, cry1A.88, encoding the Cry1 A.88 protein that has been functionally optimized using DNA shuffling techniques and based on genes derived from Bacillus thuringiensis subsp. kurstaki. The coding region which produces a 1,182-residue protein (approximately 134 kDa; SEQ ID NO: 9) is controlled by a truncated version of the promoter from Banana Streak Virus of acuminata Vietnam strain [BSV (AV)] (Lheureux et al., 2007, Archives of Virology 152: 1409-1416; Genbank accession NC_007003.1) with a second copy of the maize Adhi intron. The terminator for the cry1A.88 cassette is a portion of the Sorghum bicolor genome containing the terminator from the actin gene (SB-actin) (Genbank accession XM_002441128.1).

The third cassette (vip3Aa20 gene cassette) contains the modified vip3A gene derived from Bacillus thuringiensis strain AB88, which encodes the insecticidal Vip3Aa20 protein (Estruch et al., 1996, PNAS 93: 5389-5394). Expression of the vip3Aa20 gene is controlled by the regulatory region of the maize polyubiquitin (ubiZM1) gene, including the promoter, the 5′ untranslated region (5′ UTR) and intron (Christensen et al., 1992, Plant Molecular Biology 18: 675-689). The terminator for the vip3Aa20 gene is the terminator sequence from the proteinase inhibitor II (pinII) gene of Solanum tuberosum (Keil et al., 1986, Nucleic Acids Research 14: 5641-5650; An et al., 1989, The Plant Cell 1: 115-122). The Vip3Aa20 protein is 789-amino acid residues in length with an approximate molecular weight of 88 kDa (SEQ ID NO: 10).

The fourth gene cassette (mo-pat gene cassette) contains a maize-optimized version of the phosphinothricin acetyl transferase gene (mo-pat) from Streptomyces viridochromogenes (Wohlleben et al., 1988, Gene 70: 25-37). The mo-patgene expresses the phosphinothricin acetyl transferase (PAT) enzyme that confers tolerance to phosphinothricin. The PAT protein is 183 amino acids in length and has an approximate molecular weight of 21 kDa (SEQ ID NO: 11). Expression of the mo-pat gene is controlled by a second copy of the ubiZM1 promoter, the 5′ UTR and intron (Christensen et al., 1992, Plant Molecular Biology 18: 675-689), in conjunction with a second copy of the pinII terminator (Keil et al., 1986, Nucleic Acids Research 14: 5641-5650; An et al., 1989, The Plant Cell 1: 115-122).

According to another embodiment of the disclosure, compositions and methods are provided for identifying a novel corn plant designated DP-032218-9. The methods are based on primers or probes which specifically recognize the 5′ and/or 3′ flanking sequence of DP-032218-9. DNA molecules are provided that comprise primer sequences that when utilized in a PCR reaction will produce amplicons unique to the transgenic event DP-032218-9. The corn plant and seed comprising these molecules is an embodiment of this disclosure. Further, kits utilizing these primer sequences for the identification of the DP-032218-9 event are provided.

An additional embodiment of the disclosure relates to the specific flanking sequence of DP-032218-9 described herein, which can be used to develop specific identification methods for DP-032218-9 in biological samples. More particularly, the disclosure relates to the 5′ and/or 3′ flanking regions of DP-032218-9 which can be used for the development of specific primers and probes. A further embodiment of the disclosure relates to identification methods for the presence of DP-032218-9 in biological samples based on the use of such specific primers or probes.

According to another embodiment of the disclosure, methods of detecting the presence of DNA corresponding to the corn event DP-032218-9 in a sample are provided.

Such methods comprise: (a) contacting the sample comprising DNA with a DNA primer set, that when used in a nucleic acid amplification reaction with genomic DNA extracted from corn event DP-032218-9 produces an amplicon that is diagnostic for corn event DP-032218-9; (b) performing a nucleic acid amplification reaction, thereby producing the amplicon; and (c) detecting the amplicon.

According to another embodiment of the disclosure, methods of detecting the presence of a DNA molecule corresponding to the DP-032218-9 event in a sample, such methods comprising: (a) contacting the sample comprising DNA extracted from a corn plant with a DNA probe molecule that hybridizes under stringent hybridization conditions with DNA extracted from corn event DP-032218-9 and does not hybridize under the stringent hybridization conditions with a control corn plant DNA; (b) subjecting the sample and probe to stringent hybridization conditions; and (c) detecting hybridization of the probe to the DNA. More specifically, a method for detecting the presence of a DNA molecule corresponding to the DP-032218-9 event in a sample, such methods, consisting of (a) contacting the sample comprising DNA extracted from a corn plant with a DNA probe molecule that consists of sequences that are unique to the event, e.g. junction sequences, wherein said DNA probe molecule hybridizes under stringent hybridization conditions with DNA extracted from corn event DP-032218-9 and does not hybridize under the stringent hybridization conditions with a control corn plant DNA; (b) subjecting the sample and probe to stringent hybridization conditions; and (c) detecting hybridization of the probe to the DNA.

In addition, a kit and methods for identifying event DP-032218-9 in a biological sample which detects a DP-032218-9 specific region are provided.

DNA molecules are provided that comprise at least one junction sequence of DP-032218-9; wherein a junction sequence spans the junction between heterologous DNA inserted into the genome and the DNA from the corn cell flanking the insertion site, i.e. flanking DNA, and is diagnostic for the DP-032218-9 event.

According to another embodiment of the disclosure, methods of producing an insect resistant corn plant that comprise the steps of: (a) sexually crossing a first parental corn line comprising the expression cassettes of the disclosure, which confers resistance to insects, and a second parental corn line that lacks insect resistance, thereby producing a plurality of progeny plants; and (b) selecting a progeny plant that is insect resistant. Such methods may optionally comprise the further step of back-crossing the progeny plant to the second parental corn line to producing a true-breeding corn plant that is insect resistant.

A further embodiment of the disclosure provides a method of producing a corn plant that is resistant to insects comprising transforming a corn cell with the DNA construct PHP36676, growing the transformed corn cell into a corn plant, selecting the corn plant that shows resistance to insects, and further growing the corn plant into a fertile corn plant. The fertile corn plant can be self-pollinated or crossed with compatible corn varieties to produce insect resistant progeny. In some embodiments the event DP-032218-9 was generated by transforming the maize line PHWWE with plasmid PHP36676.

Another embodiment of the disclosure further relates to a DNA detection kit for identifying maize event DP-032218-9 in biological samples. The kit comprises a first primer which specifically recognizes the 5′ or 3′ flanking region of DP-032218-9, and a second primer which specifically recognizes a sequence within the foreign DNA of DP-032218-9, or within the flanking DNA, for use in a PCR identification protocol. A further embodiment of the disclosure relates to a kit for identifying event DP-032218-9 in biological samples, which kit comprises a specific probe having a sequence which corresponds or is complementary to, a sequence having between 80% and 100% sequence identity with a specific region of event DP-032218-9. The sequence of the probe corresponds to a specific region comprising part of the 5′ or 3′ flanking region of event DP-032218-9.

The methods and kits encompassed by the embodiments of the present disclosure can be used for different purposes such as, but not limited to the following: to identify event DP-032218-9 in plants, plant material or in products such as, but not limited to, food or feed products (fresh or processed) comprising, or derived from plant material; additionally or alternatively, the methods and kits can be used to identify transgenic plant material for purposes of segregation between transgenic and non-transgenic material; additionally or alternatively, the methods and kits can be used to determine the quality of plant material comprising maize event DP-032218-9. The kits may also contain the reagents and materials necessary for the performance of the detection method.

A further embodiment of this disclosure relates to the DP-032218-9 corn plant or its parts, including, but not limited to, pollen, ovules, vegetative cells, the nuclei of pollen cells, and the nuclei of egg cells of the corn plant DP-032218-9 and the progeny derived thereof. The corn plant and seed of DP-032218-9 from which the DNA primer molecules provide a specific amplicon product is an embodiment of the disclosure.

The following embodiments are encompassed by the present disclosure.

1. A DNA construct comprising:

(a) a first expression cassette, comprising in operable linkage:
(i) a full length Citrus Yellow Mosaic virus (CYMV) promoter;
(ii) a maize adh1 first intron;
(iii) a synthetic chloroplast targeting peptide
(iv) a Cry2A.127 encoding DNA molecule; and
(v) a ubiquitin3 (UBQ3) transcriptional terminator; and
(vi) a 3′ untranslated region of an Arabidopsis ribosomal protein gene;

(b) a second expression cassette, comprising in operable linkage:
(i) a truncated BSV promoter and second adh1 intron;
(ii) a Cry1A.88 encoding DNA molecule; and
(iii) a sorghum actin transcriptional terminator;

(c) a third expression cassette, comprising in operable linkage:
(i) a maize polyubiquitin promoter;
(ii) a 5′ untranslated region and intronl of a maize polyubiquitin gene;
(iii) a Vip3Aa20 encoding DNA molecule; and
(iv) a pinII transcriptional terminator; and

(d) a fourth expression cassette, comprising in operable linkage
(i) a maize polyubiquitin promoter;
(ii) a mo-pat encoding DNA molecule; and
(iii) a pinII transcriptional terminator.
   2. A plant comprising the DNA construct of embodiment 1.
   3. A plant of embodiment 2, wherein said plant is a corn plant.
   4. A plant comprising the sequence set forth in SEQ ID NO: 5.
   5. A corn plant comprising the genotype of the corn event DP-032218-9, wherein said genotype comprises the nucleotide sequences at the junction of the insert and genomic sequence as set forth in the forward and reverse junction primers.
   6. The corn plant of embodiment 5, wherein said genotype comprises the nucleotide sequence set forth in the forward primer.
   7. The corn plant of embodiment 5, wherein said genotype comprises the nucleotide sequence set forth in the reverse primer.
   8. A corn event DP-032218-9, wherein a representative sample of seed of said corn event has been deposited with American Type Culture Collection (ATCC) with Accession No. PTA-13391.
   9. Plant parts of the corn event of embodiment 8.
   10. Seed comprising corn event DP-032218-9, wherein said seed comprises a DNA molecule selected from the group consisting of a forward junction primer and a reverse junction primer, wherein a representative sample of corn event DP-032218-9 seed of has been deposited with American Type Culture Collection (ATCC) with Accession No. PTA-13391.
   11. A corn plant, or part thereof, grown from the seed of embodiment 10.
   12. A transgenic seed produced from the corn plant of embodiment 11 comprising event DP-032218-9.
   13. A transgenic corn plant, or part thereof, grown from the seed of embodiment
   14. An isolated nucleic acid molecule comprising a nucleotide sequence selected from the group consisting of SEQ ID NO: 1, SEQ ID NO: 5, a DP-032218-9 event specific forward junction primer, a DP-032218-9 event specific reverse junction primer, a DP-032218-9 event specific amplicon, and full length complements thereof.
   15. A DP-032218-9 event specific amplicon comprising the nucleic acid sequence selected from the group consisting of a DP-032218-9 event specific forward junction primer, a DP-032218-9 event specific reverse junction primer and full length complements thereof.
   16. A biological sample derived from corn event DP-032218-9 plant, tissue, or seed, wherein said sample comprises a nucleotide sequence which is or is complementary to a sequence selected from the group consisting of a forward junction primer and a reverse junction primer, wherein said nucleotide sequence is detectable in said sample using a nucleic acid amplification or nucleic acid hybridization method, wherein a representative sample of said corn event DP-032218-9 seed of has been deposited with American Type Culture Collection (ATCC) with Accession No. PTA-13391.
   17. The biological sample of embodiment 16, wherein said biological sample comprise plant, tissue, or seed of transgenic corn event DP-032218-9.
   18. The biological sample of embodiment 17, wherein said biological sample is a DNA sample extracted from the transgenic corn plant event DP-032218-9, and wherein said DNA sample comprises one or more of the nucleotide sequences selected from the group consisting of a forward junction primer, a reverse junction primer, and the complement thereof.
   19. The biological sample of embodiment 18, wherein said biological sample is selected from the group consisting of corn flour, corn meal, corn syrup, corn oil, corn starch, and cereals manufactured in whole or in part to contain corn by-products.
   20. An extract derived from corn event DP-032218-9 plant, tissue, or seed and comprising a nucleotide sequence which is or is complementary to a sequence selected from the group consisting of a forward junction primer and a reverse junction primer, wherein a representative sample of said corn event DP-032218-9 seed has been deposited with American Type Culture Collection (ATCC) with Accession No. PTA-13391.
   21. The extract of embodiment 20, wherein said nucleotide sequence is detectable in said extract using a nucleic acid amplification or nucleic acid hybridization method.
   22. The extract of embodiment 21, wherein said extract comprises plant, tissue, or seed of transgenic corn plant event DP-032218-9.
   23. The extract of embodiment 22, further comprising a composition selected from the group consisting of corn flour, corn meal, corn syrup, corn oil, corn starch, and cereals manufactured in whole or in part to contain corn by-products, wherein said composition comprises a detectable amount of said nucleotide sequence.
   24. A method of producing hybrid corn seeds comprising:
(a) planting seeds of a first inbred corn line comprising a nucleotide sequence selected from the group consisting of a forward junction primer, a reverse junction primer, and seeds of a second inbred line having a different genotype;
(b) cultivating corn plants resulting from said planting until time of flowering;
(c) emasculating said flowers of plants of one of the corn inbred lines;
(d) sexually crossing the two different inbred lines with each other; and
(e) harvesting the hybrid seed produced thereby.
   25. The method according to embodiment 24, wherein the plants of the first inbred corn line are the female parents.
   26. The method according to embodiment 24, wherein the plants of first inbred corn line are the male parents.
   27. A method for producing a corn plant resistant to lepidopteran pests comprising:
(a) sexually crossing a first parent corn plant with a second parent corn plant, wherein said first or second parent corn plant comprises event DP-032218-9 DNA, thereby producing a plurality of first generation progeny plants;
(b) selecting a first generation progeny plant that is resistant to lepidopteran insect infestation;
(c) selfing the first generation progeny plant, thereby producing a plurality of second generation progeny plants; and
(d) selecting from the second generation progeny plants, a plant that is resistant to lepidopteran pests;
   wherein the second generation progeny plants comprise the DNA construct according to embodiment 1.
   28. A method of producing hybrid corn seeds comprising:
(a) planting seeds of a first inbred corn line comprising the DNA construct of embodiment 1 and seeds of a second inbred line having a genotype different from the first inbred corn line;
(b) cultivating corn plants resulting from said planting until time of flowering;
(c) emasculating said flowers of plants of one of the corn inbred lines;
(d) sexually crossing the two different inbred lines with each other; and
(e) harvesting the hybrid seed produced thereby.
   29. The method of embodiment 28 further comprising the step of backcrossing the second generation progeny plant of step (d) that comprises corn event DP-032218-9 DNA to the parent plant that lacks the corn event DP-032218-9 DNA, thereby producing a backcross progeny plant that is resistant to at least lepidopteran insects.
   30. A method for producing a corn plant resistant to at least lepidopteran insects, said method comprising:
(a) sexually crossing a first parent corn plant with a second parent corn plant, wherein said first or second parent corn plant is a corn event DP-032218-9 plant, thereby producing a plurality of first generation progeny plants;
(b) selecting a first generation progeny plant that is resistant to at least lepidopteran insects infestation;
(c) backcrossing the first generation progeny plant of step (b) with the parent plant that lacks corn event DP-032218-9 DNA, thereby producing a plurality of backcross progeny plants; and
(d) selecting from the backcross progeny plants, a plant that is resistant to at least lepidopteran insects infestation;
   wherein the selected backcross progeny plant of step (d) comprises SEQ ID NO: 5.
   31. The method according to embodiment 28, wherein the plants of the first inbred corn line are the female parents or male parents.
   32. Hybrid seed produced by the method of embodiment 28.
   33. A method of determining zygosity of DNA of a corn plant comprising corn event DP-032218-9 in a biological sample comprising:
(a) contacting said sample with a first primer selected from the group consisting of one or more forward junction primer sequences, and a second primer selected from the group consisting of one or more reverse junction primer sequences, such that
   (1) when used in a nucleic acid amplification reaction comprising corn event DP-032218-9 DNA, produces a first amplicon that is diagnostic for corn event, DP-032218-9 and
   (2) when used in a nucleic acid amplification reaction comprising corn genomic DNA other than DP-032218-9 DNA, produces a second amplicon that is diagnostic for corn genomic DNA other than DP-032218-9 DNA;
(b) performing a nucleic acid amplification reaction; and
(c) detecting the amplicons so produced, wherein detection of presence of both amplicons indicates that said sample is heterozygous for corn event DP-032218-9 DNA, wherein detection of only the first amplicon indicates that said sample is homozygous for corn event DP-032218-9 DNA.
   34. A method of detecting the presence of a nucleic acid molecule that is unique to event DP-032218-9 in a sample comprising corn nucleic acids, the method comprising:
(a) contacting the sample with a pair of primers that, when used in a nucleic-acid amplification reaction with genomic DNA from event DP-032218-9 produces an amplicon that is diagnostic for event DP-032218-9;
(b) performing a nucleic acid amplification reaction, thereby producing the amplicon; and
(c) detecting the amplicon.
   35. A pair of polynucleotide primers comprising a first polynucleotide primer and a second polynucleotide primer which function together in the presence of event DP-032218-9 DNA template in a sample to produce an amplicon diagnostic for event DP-032218-9.
   36. The pair of polynucleotide primers according to embodiment 35, wherein the sequence of the first polynucleotide primer is or is complementary to a corn plant genome sequence flanking the point of insertion of a heterologous DNA sequence inserted into the corn plant genome of event DP-032218-9, and the sequence of the second polynucleotide primer is or is complementary to the heterologous DNA sequence inserted into the genome of event DP-032218-9.
   37. A method of detecting the presence of DNA corresponding to the DP-032218-9 event in a sample, the method comprising:
(a) contacting the sample comprising maize DNA with a polynucleotide probe that hybridizes under stringent hybridization conditions with DNA from maize event DP-032218-9 and does not hybridize under said stringent hybridization conditions with a non-DP-032218-9 maize plant DNA;
(b) subjecting the sample and probe to stringent hybridization conditions; and
(c) detecting hybridization of the probe to the DNA;
   wherein detection of hybridization indicates the presence of the DP-032218-9 event.
   38. A kit for detecting nucleic acids that are unique to event DP-032218-9 comprising at least one nucleic acid molecule of sufficient length of contiguous polynucleotides to function as a primer or probe in a nucleic acid detection method, and which upon amplification of or hybridization to a target nucleic acid sequence in a sample followed by detection of the amplicon or hybridization to the target sequence, are diagnostic for the presence of nucleic acid sequences unique to event DP-032218-9 in the sample.
   39. The kit according to embodiment 42, wherein the nucleic acid molecule comprises a nucleotide sequence from SEQ ID NO: 5.
   40. The kit according to embodiment 43, wherein the nucleic acid molecule is a primer selected from the group consisting of one or more junction primer sequences, and the complements thereof.
   41. A method of detecting in a sample comprising corn nucleic acids the presence of a nucleic acid molecule unique to event DP-032218-9, the method comprising:
(a) contacting the sample with a first polynucleotide primer of SEQ ID NO: 2 and a second polynucleotide primer of SEQ ID NO: 3;
(b) performing a nucleic acid amplification reaction, thereby producing an amplicon of SEQ ID NO: 12; and
(c) detecting the amplicon using an DP-032218-9 event specific probe of SEQ ID NO: 4.
   42. The method of embodiment 41, wherein the method further comprises contacting the sample with a first polynucleotide primer unique to maize HMG of SEQ ID NO: 13 and a second polynucleotide primer unique to maize HMG of SEQ ID NO: 14; and detecting the amplicon of SEQ ID NO: 16 with a maize HGM specific probe of SEQ ID NO: 15, wherein the HGM amplification serves to determine the quality of the nucleic acid in the sample.
   43. The method of embodiment 41 or 42, wherein the amplification method is a real-time PCR method.
   44. The method of any one of embodiments 41, 42 or 43, wherein the real-time PCR is performed in thermo-cycler instrument.
   45. The method of any one of embodiments 41, 42, 43 or 44, wherein the probe is attached to a conventional detectable label, reporter molecule, and/or quencher molecule.
   46. The method of embodiment 45, wherein the detectable label is a fluorescent dye.
   47. The method of embodiment 46, wherein the fluorescent dye is selected from FAM™ and VIC®.
   48. The method of any one of embodiments 45, 46, or 47, wherein the quencher molecule is a non-fluorescent quencher dye attached to a minor groove binding moiety.
   49. A kit for detecting nucleic acids that are unique to event DP-032218-9 comprising the nucleic acid molecules of SEQ ID NO: 2, SEQ ID NO: 3 and SEQ ID NO: 4.
   50. A kit for detecting nucleic acids that are unique to event DP-032218-9 comprising SEQ ID NO: 12.
   51. A method for determining in a sample comprising corn nucleic acids the zygosity of a nucleic acid unique to event DP-032218-9, the method comprising:
(a) contacting the sample with a first polynucleotide primer unique to event DP-032218-9 of SEQ ID NO: 2 and a second polynucleotide primer unique to event DP-032218-9 of SEQ ID NO: 3;
(b) contacting the sample with a first polynucleotide primer unique to maize HMG of SEQ ID NO: 13 and a second polynucleotide primer unique to maize HMG of SEQ ID NO: 14;
(c) performing a nucleic acid amplification reaction, thereby producing an amplicon unique to detecting the amplicon of SEQ ID NO: 12 and an amplicon unique to maize HMG of SEQ ID NO: 16;
(d) detecting the amplicon of SEQ ID NO: 12 with tan event DP-032218-9 specific probe of SEQ ID NO: 4;
(e) detecting the amplicon of SEQ ID NO: 16 with a maize HMG specific probe of SEQ ID NO: 15; and
(f) determining the zygosity of the DP-032218-9 event.
   52. The method of embodiment 51, wherein the amplification method is a real-time PCR method.
   53. The method of embodiment 51 or 52, wherein the real-time PCR is performed in thermo-cycler instrument.
   54. The method of any one of embodiments 51, 52 or 53, wherein the probes are attached to a conventional detectable label, reporter molecule and/or quencher molecule.
   55. The method of embodiment 54, wherein the detectable label is a fluorescent dye.
   56. The method of embodiment 55, wherein the fluorescent dye is selected from FAM™ and VIC®.
   57. The method of embodiment 54, 55 or 66, wherein the quencher molecule is a non-fluorescent quencher dye attached to a minor groove binding moiety.
   58. The method of any one of embodiments 51 to 57, wherein the zygosity is determined by the 2^{-ΔΔC}_{T} method.
   59. A kit for determining in a sample comprising corn nucleic acids the zygosity of a nucleic acid unique to event DP-032218-9 comprising SEQ ID NO: 2, SEQ ID NO: 3, SEQ ID NO: 4, SEQ ID NO: 13, SEQ ID NO: 14 and SEQ ID NO: 15.

### BRIEF DESCRIPTION OF THE DRAWINGS

FIG. 1 shows a schematic diagram of plasmid PHP36676 with genetic elements indicated.

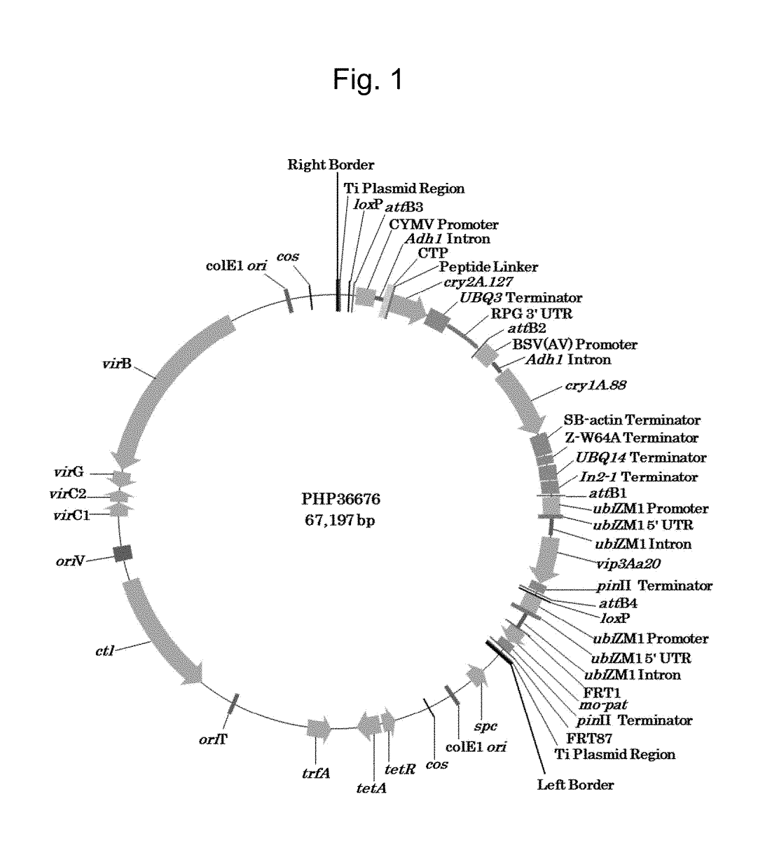

FIG. 2 shows a schematic diagram of the T-DNA region from plasmid PHP36676 with the identification of the cry2A.127, cry1A.88, vip3Aa20, and mo-patgene cassettes. The size of the T-DNA is 24,266 base pairs.

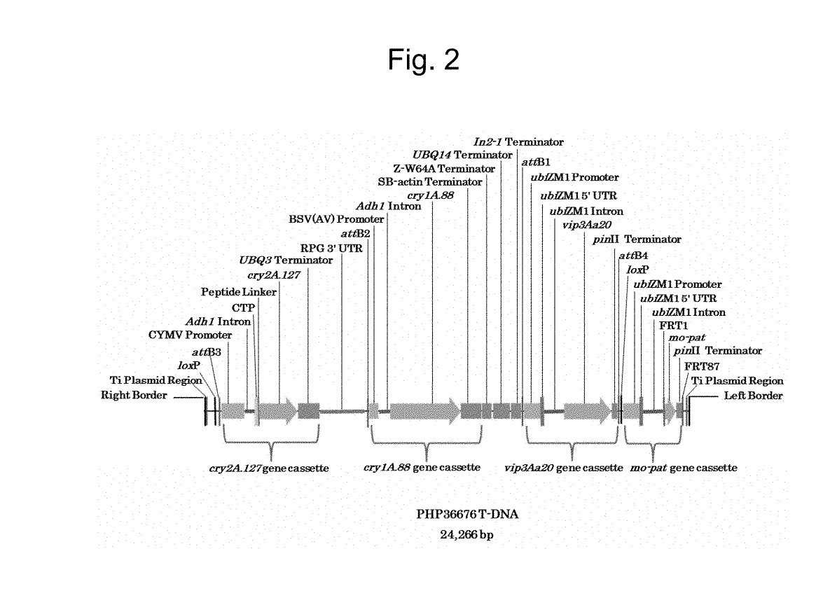

### DETAILED DESCRIPTION

This disclosure relates to the insect resistant corn (Zea mays) plant DP-032218-9, also referred to as “maize line DP-032218-9,” “maize event DP-032218-9,” and “032218 maize,” and to the DNA plant expression construct of corn plant DP-032218-9 and the detection of the transgene/flanking insertion region in corn plant DP-032218-9 and progeny thereof.

According to one embodiment of the disclosure, compositions and methods are provided for identifying a novel corn plant designated DP-032218-9. The methods are based on primers or probes which specifically recognize the 5′ and/or 3′ flanking sequence of DP-032218-9. DNA molecules are provided that comprise primer sequences that when utilized in a PCR reaction will produce amplicons unique to the transgenic event DP-032218-9. The corn plant and seed comprising these molecules is an embodiment of this disclosure. Further, kits utilizing these primer sequences for the identification of the DP-032218-9 event are provided.

An additional embodiment of the disclosure relates to the specific flanking sequence of DP-032218-9 described herein, which can be used to develop specific identification methods for DP-032218-9 in biological samples. More particularly, the disclosure relates to the 5′ and/or 3′ flanking regions of DP-032218-9 which can be used for the development of specific primers and probes. A further embodiment of the disclosure relates to identification methods for the presence of DP-032218-9 in biological samples based on the use of such specific primers or probes.

According to another embodiment of the disclosure, methods of detecting the presence of DNA corresponding to the corn event DP-032218-9 in a sample are provided.

Such methods comprise: (a) contacting the sample comprising DNA with a DNA primer set, that when used in a nucleic acid amplification reaction with genomic DNA extracted from corn event DP-032218-9 produces an amplicon that is diagnostic for corn event DP-032218-9; (b) performing a nucleic acid amplification reaction, thereby producing the amplicon; and (c) detecting the amplicon.

According to another embodiment of the disclosure, methods of detecting the presence of a DNA molecule corresponding to the DP-032218-9 event in a sample, such methods comprising: (a) contacting the sample comprising DNA extracted from a corn plant with a DNA probe molecule that hybridizes under stringent hybridization conditions with DNA extracted from corn event DP-032218-9 and does not hybridize under the stringent hybridization conditions with a control corn plant DNA; (b) subjecting the sample and probe to stringent hybridization conditions; and (c) detecting hybridization of the probe to the DNA. More specifically, a method for detecting the presence of a DNA molecule corresponding to the DP-032218-9 event in a sample, such methods, consisting of (a) contacting the sample comprising DNA extracted from a corn plant with a DNA probe molecule that consists of sequences that are unique to the event, e.g. junction sequences, wherein said DNA probe molecule hybridizes under stringent hybridization conditions with DNA extracted from corn event DP-032218-9 and does not hybridize under the stringent hybridization conditions with a control corn plant DNA; (b) subjecting the sample and probe to stringent hybridization conditions; and (c) detecting hybridization of the probe to the DNA.

In addition, a kit and methods for identifying event DP-032218-9 in a biological sample which detects a DP-032218-9 specific region are provided.

DNA molecules are provided that comprise at least one junction sequence of DP-032218-9; wherein a junction sequence spans the junction between heterologous DNA inserted into the genome and the DNA from the corn cell flanking the insertion site, i.e. flanking DNA, and is diagnostic for the DP-032218-9 event.

According to another embodiment of the disclosure, methods of producing an insect resistant corn plant that comprise the steps of: (a) sexually crossing a first parental corn line comprising the expression cassettes of the disclosure, which confers resistance to insects, and a second parental corn line that lacks insect resistance, thereby producing a plurality of progeny plants; and (b) selecting a progeny plant that is insect resistant. Such methods may optionally comprise the further step of back-crossing the progeny plant to the second parental corn line to producing a true-breeding corn plant that is insect resistant.

A further embodiment of the disclosure provides a method of producing a corn plant that is resistant to insects comprising transforming a corn cell with the DNA construct PHP36676, growing the transformed corn cell into a corn plant, selecting the corn plant that shows resistance to insects, and further growing the corn plant into a fertile corn plant. The fertile corn plant can be self-pollinated or crossed with compatible corn varieties to produce insect resistant progeny.

Another embodiment of the disclosure further relates to a DNA detection kit for identifying maize event DP-032218-9 in biological samples. The kit comprises a first primer which specifically recognizes the 5′ or 3′ flanking region of DP-032218-9, and a second primer which specifically recognizes a sequence within the foreign DNA of DP-032218-9, or within the flanking DNA, for use in a PCR identification protocol. A further embodiment of the disclosure relates to a kit for identifying event DP-032218-9 in biological samples, which kit comprises a specific probe having a sequence which corresponds or is complementary to, a sequence having between 80% and 100% sequence identity with a specific region of event DP-032218-9. The sequence of the probe corresponds to a specific region comprising part of the 5′ or 3′ flanking region of event DP-032218-9.

The methods and kits encompassed by the embodiments of the present disclosure can be used for different purposes such as, but not limited to the following: to identify event DP-032218-9 in plants, plant material or in products such as, but not limited to, food or feed products (fresh or processed) comprising, or derived from plant material; additionally or alternatively, the methods and kits can be used to identify transgenic plant material for purposes of segregation between transgenic and non-transgenic material; additionally or alternatively, the methods and kits can be used to determine the quality of plant material comprising maize event DP-032218-9. The kits may also contain the reagents and materials necessary for the performance of the detection method.

A further embodiment of this disclosure relates to the DP-032218-9 corn plant or its parts, including, but not limited to, pollen, ovules, vegetative cells, the nuclei of pollen cells, and the nuclei of egg cells of the corn plant DP-032218-9 and the progeny derived thereof. The corn plant and seed of DP-032218-9 from which the DNA primer molecules provide a specific amplicon product is an embodiment of the disclosure.

Specifically, the first cassette contains the proprietary cry2A. 127 gene, a Cry2Ab-like coding sequence that has been functionally optimized using DNA shuffling and directed mutagenesis techniques. The 634 residue protein produced by expression of the cry2A. 127 sequence is targeted to maize chloroplasts through the addition of a 56 amino acid codon-optimized synthetic chloroplast targeting peptide (CTP) as well as 4 synthetic linker amino acids, resulting in a total length of 694 amino acids (approximately 77 kDa) for the precursor protein (the Cry2A.127 CTP sequence is cleaved upon insertion into the chloroplast, resulting in a mature protein of approximately 71 kDa. The expression of the cry2A. 127 gene and attached transit peptide is controlled by the full length promoter from the CYMV promoter (Citrus Yellow Mosaic Virus; Genbank accession AF347695.1) along with a downstream copy of the maize adh1 intron (Dennis et al., 1984). Transcription of the cry2A. 127 gene cassette is terminated by the downstream presence of the Arabidopsis thaliana ubiquitin 3 (UBQ3) termination region (Callis et al., 1995). In addition, a 2.2 kB fragment corresponding to the 3′ un-translated region from an Arabidopsis ribosomal protein gene (TAIR accession AT3G28500; Salanoubat et al., 2000) is located between the cry2A. 127 and cry1A.88 cassettes in order to eliminate any potential read thru transcripts.

The second cassette contains a second shuffled proprietary insect control gene, the Cry1A-like cry1A.88 coding region. This 1182 residue coding region (which produces a precursor protein of approximately 133 kDa, is controlled by a truncated version (470 nucleotides in length) of the full length promoter from Banana Streak Virus (Acuminata Vietnam strain; Lheureux et al., 2007) along with a second copy of the maize adh1 intron. The termination region for the cry1A.88 cassette is a 1.1 kB portion of the Sorghum bi-color genome containing the 3′ termination region from the SB-Actin gene (Paterson et al., 2009)). Three other termination regions are present between the second and third cassettes; the 27 kD gamma zein terminator originally isolated from maize line W64A (Das et al., 1991), a genomic fragment of Arabidopsis thaliana chromosome 4 containing the Ubiquitin-14 (UBQ14) 3′UTR and terminator (Mayer et al., 1999) and the termination sequence from the maize 1n2-1 gene (Hershey and Stoner, 1991).

The third cassette contains the vip3Aa20 gene, which codes for a synthetic version of the insecticidal Vip3Aa20 protein (present in the approved Syngenta event MIR162; Estruch et al., 1996). Expression of the vip3Aa20 gene is controlled by the maize polyubiquitin promoter, including the 5′ untranslated region and intron 1 (Christensen et al., 1992). The terminator for the vip3Aa20 gene is the 3′ terminator sequence from the proteinase inhibitor II gene of Solanum tuberosum (pinII terminator) (Keil et al., 1986; An et al., 1989). The Vip3Aa20 protein is 789 amino acid residues in length with an approximate molecular weight of 88 kDa.

The fourth and final gene cassette contains a version of the phosphinothricin acetyl transferase gene (mo-pat) from Streptomyces viridochromogenes (Wohlleben et al., 1988) that has been optimized for expression in maize. The pat gene expresses the phosphinothricin acetyl transferase enzyme (PAT) that confers tolerance to phosphinothricin. The PAT protein is 183 amino acids residues in length and has a molecular weight of approximately 21 kDa. Expression of the mo-patgene is controlled by a second copy of the maize polyubiquitin promoter/5′UTR/intron in conjunction with a second copy of the pinII terminator. Plants containing the DNA constructs are also provided. A description of the genetic elements in the PHP36676 T-DNA (set forth in SEQ ID NO: 1) and their sources are described further in the Table of Abbreviations below.

The following definitions and methods are provided to better define the present disclosure and to guide those of ordinary skill in the art in the practice of the present disclosure. Unless otherwise noted, terms are to be understood according to conventional usage by those of ordinary skill in the relevant art. Definitions of common terms in molecular biology may also be found in Rieger et al., Glossary of Genetics: Classical and Molecular, 5^{th} edition, Springer-Verlag; New York, 1991; and Lewin, Genes V, Oxford University Press: New York, 1994. The nomenclature for DNA bases as set forth at 37 CFR § 1.822 is used.

The following table sets forth abbreviations used throughout this document, and in particular in the Examples section.

Table of Abbreviations

| | |
|---|---|
| 032218 maize | Maize containing event DP-032218-9 |
| Bp | Base pair |
| BSV | Banana Streak Virus |
| Bt | Bacillus thuringiensis |
| cry2A.127 | cry2A.127-like coding sequence functionally |
| | optimized using DNA shuffling and directed |
| | mutagenesis techniques |
| Cry2A.127 | Protein from cry2A.127 gene |
| cry1A.88 | cry1A.88-like coding sequence (including protoxin |
| | regions) functionally optimized using DNA shuffling |
| | and directed mutagenesis techniques |
| Cry1A.88 | Protein from cry1A.88 gene |
| CYMV | Citrus Yellow Mosaic Virus |
| kb | Kilobase pair |
| kDa | KiloDalton |
| LB | Left T-DNA border |
| mo-pat | Maize-optimized version of the phosphinothricin acetyl |
| | transferase gene (pat) from Streptomyces |
| | viridochromgenes |
| MO-PAT | Protein from phosphinothricin acetyl transferase gene |
| PCR | Polymerase chain reaction |
| pinII | Proteinase inhibitor II gene from Solanum tuberosum |
| RB | Right T-DNA border |
| T-DNA | The transfer DNA portion of the Agrobacterium |
| | transformation plasmid between the Left and Right |
| | Borders that is expected to be transferred to the |
| | plant genome |
| UBQ3 | ubiquitin 3 gene of Arabidopsis thaliana |
| ubiZM1 | Promoter region from Zea mays polyubiquitin gene |
| UTR | Untranslated region |
| vip3Aa20 | Synthetic vip3Aa20 gene (present in approved Syngenta |
| | event MIR162) |
| Vip3Aa20 | Protein from vip3Aa20 gene |
| ECB | European corn borer (Ostrinia nubilalis) |
| FAW | Fall armyworm (Spodoptera frugiperda) |
| CEW | Corn earworm |

Compositions of this disclosure include seed deposited as Patent Deposit No. PTA-13391 and plants, plant cells, and seed derived therefrom. Applicant(s) have made a deposit of at least 2500 seeds of maize event DP-032218-9 with the American Type Culture Collection (ATCC), Manassas, Va. 20110-2209 USA, on Dec. 12, 2012 and the deposits were assigned ATCC Deposit No. PTA-13391. These deposits will be maintained under the terms of the Budapest Treaty on the International Recognition of the Deposit of Microorganisms for the Purposes of Patent Procedure. These deposits were made merely as a convenience for those of skill in the art and are not an admission that a deposit is required under 35 U.S.C. § 112. The seeds deposited with the ATCC on Dec. 12, 2012 were taken from the deposit maintained by Pioneer Hi-Bred International, Inc., 7250 NW 62^{nd} Avenue, Johnston, Iowa 50131-1000. Access to this deposit will be available during the pendency of the application to the Commissioner of Patents and Trademarks and persons determined by the Commissioner to be entitled thereto upon request. Upon allowance of any claims in the application, the Applicant(s) will make available to the public, pursuant to 37 C.F.R. § 1.808, sample(s) of the deposit of at least 2500 seeds of hybrid maize with the American Type Culture Collection (ATCC), 10801 University Boulevard, Manassas, Va. 20110-2209. This deposit of seed of maize event DP-032218-9 will be maintained in the ATCC depository, which is a public depository, for a period of 30 years, or 5 years after the most recent request, or for the enforceable life of the patent, whichever is longer, and will be replaced if it becomes nonviable during that period. Additionally, Applicant(s) have satisfied all the requirements of 37 C.F.R. §§ 1.801-1.809, including providing an indication of the viability of the sample upon deposit. Applicant(s) have no authority to waive any restrictions imposed by law on the transfer of biological material or its transportation in commerce. Applicant(s) do not waive any infringement of their rights granted under this patent or rights applicable to event DP-032218-9 under the Plant Variety Protection Act (7 USC 2321 et seq.). Unauthorized seed multiplication prohibited. The seed may be regulated.

As used herein, the term “comprising” means “including but not limited to.”

As used herein, the term “corn” means Zea mays or maize and includes all plant varieties that can be bred with corn, including wild maize species.

As used herein, the term “DP-032218-9 specific” refers to a nucleotide sequence which is suitable for discriminatively identifying event DP-032218-9 in plants, plant material, or in products such as, but not limited to, food or feed products (fresh or processed) comprising, or derived from plant material.

As used herein, the terms “insect resistant” and “impacting insect pests” refers to effecting changes in insect feeding, growth, and/or behavior at any stage of development, including but not limited to: killing the insect; retarding growth; preventing reproductive capability; inhibiting feeding; and the like.

As used herein, the terms “pesticidal activity” and “insecticidal activity” are used synonymously to refer to activity of an organism or a substance (such as, for example, a protein) that can be measured by numerous parameters including, but not limited to, pest mortality, pest weight loss, pest attraction, pest repellency, and other behavioral and physical changes of a pest after feeding on and/or exposure to the organism or substance for an appropriate length of time. For example “pesticidal proteins” are proteins that display pesticidal activity by themselves or in combination with other proteins.

“Coding sequence” refers to a nucleotide sequence that codes for a specific amino acid sequence. As used herein, the terms “encoding” or “encoded” when used in the context of a specified nucleic acid mean that the nucleic acid comprises the requisite information to guide translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature.

Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. “Foreign” refers to material not normally found in the location of interest. Thus “foreign DNA” may comprise both recombinant DNA as well as newly introduced, rearranged DNA of the plant. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure. The site in the plant genome where a recombinant DNA has been inserted may be referred to as the “insertion site” or “target site”.

As used herein, “insert DNA” refers to the heterologous DNA within the expression cassettes used to transform the plant material while “flanking DNA” can exist of either genomic DNA naturally present in an organism such as a plant, or foreign (heterologous) DNA introduced via the transformation process which is extraneous to the original insert DNA molecule, e.g. fragments associated with the transformation event. A “flanking region” or “flanking sequence” as used herein refers to a sequence of at least 20 bp, preferably at least 50 bp, and up to 5000 bp, which is located either immediately upstream of and contiguous with or immediately downstream of and contiguous with the original foreign insert DNA molecule. Transformation procedures leading to random integration of the foreign DNA will result in transformants containing different flanking regions characteristic and unique for each transformant. When recombinant DNA is introduced into a plant through traditional crossing, its flanking regions will generally not be changed. Transformants will also contain unique junctions between a piece of heterologous insert DNA and genomic DNA, or two (2) pieces of genomic DNA, or two (2) pieces of heterologous DNA. A “junction” is a point where two (2) specific DNA fragments join. For example, a junction exists where insert DNA joins flanking DNA. A junction point also exists in a transformed organism where two (2) DNA fragments join together in a manner that is modified from that found in the native organism. “Junction DNA” refers to DNA that comprises a junction point. Two junction sequences set forth in this disclosure are the junction point between the maize genomic DNA and the 5′ end of the insert as set forth in the forward junction sequences and the junction point between the 3′ end of the insert and maize genomic DNA as set forth in the reverse junction sequences.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous nucleotide sequence can be from a species different from that from which the nucleotide sequence was derived, or, if from the same species, the promoter is not naturally found operably linked to the nucleotide sequence. A heterologous protein may originate from a foreign species, or, if from the same species, is substantially modified from its original form by deliberate human intervention.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences can include, without limitation: promoters, translation leader sequences, introns, and polyadenylation recognition sequences.

“Promoter” refers to a nucleotide sequence capable of controlling the expression of a coding sequence or functional RNA. In general, a coding sequence is located 3′ to a promoter sequence. The promoter sequence consists of proximal and more distal upstream elements, the latter elements are often referred to as enhancers. Accordingly, an “enhancer” is a nucleotide sequence that can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic nucleotide segments. It is understood by those skilled in the art that different promoters may direct the expression of a gene in different tissues or cell types, or at different stages of development, or in response to different environmental conditions. Promoters that cause a nucleic acid fragment to be expressed in most cell types at most times are commonly referred to as “constitutive promoters”. New promoters of various types useful in plant cells are constantly being discovered; numerous examples may be found in the compilation by Okamuro and Goldberg (1989) Biochemistry of Plants 15:1-82. It is further recognized that since in most cases the exact boundaries of regulatory sequences have not been completely defined, nucleic acid fragments of different lengths may have identical promoter activity.

The “translation leader sequence” refers to a nucleotide sequence located between the promoter sequence of a gene and the coding sequence. The translation leader sequence is present in the fully processed mRNA upstream of the translation start sequence. The translation leader sequence may affect numerous parameters including, but not limited to, processing of the primary transcript to mRNA, mRNA stability and/or translation efficiency. Examples of translation leader sequences have been described (Turner and Foster (1995) Mol. Biotechnol. 3:225-236).

The “3′ non-coding sequences” refer to nucleotide sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (1989) Plant Cell 1:671-680.

A “protein” or “polypeptide” is a chain of amino acids arranged in a specific order determined by the coding sequence in a polynucleotide encoding the polypeptide.

A DNA construct is an assembly of DNA molecules linked together that provide one or more expression cassettes. The DNA construct may be a plasmid that is enabled for self-replication in a bacterial cell and contains various endonuclease enzyme restriction sites that are useful for introducing DNA molecules that provide functional genetic elements, i.e., promoters, introns, leaders, coding sequences, 3′ termination regions, among others; or a DNA construct may be a linear assembly of DNA molecules, such as an expression cassette. The expression cassette contained within a DNA construct comprises the necessary genetic elements to provide transcription of a messenger RNA. The expression cassette can be designed to express in prokaryote cells or eukaryotic cells. Expression cassettes of the embodiments of the present disclosure are designed to express in plant cells.

The DNA molecules of embodiments of the disclosure are provided in expression cassettes for expression in an organism of interest. The cassette will include 5′ and 3′ regulatory sequences operably linked to a coding sequence. “Operably linked” means that the nucleic acid sequences being linked are contiguous and, where necessary to join two protein coding regions, contiguous and in the same reading frame. Operably linked is intended to indicate a functional linkage between a promoter and a second sequence, wherein the promoter sequence initiates and mediates transcription of the DNA sequence corresponding to the second sequence. The cassette may additionally contain at least one additional gene to be co-transformed into the organism. Alternatively, the additional gene(s) can be provided on multiple expression cassettes or multiple DNA constructs.

The expression cassette will include in the 5′ to 3′ direction of transcription: a transcriptional and translational initiation region, a coding region, and a transcriptional and translational termination region functional in the organism serving as a host. The transcriptional initiation region (i.e., the promoter) may be native or analogous, or foreign or heterologous to the host organism. Additionally, the promoter may be the natural sequence or alternatively a synthetic sequence. The expression cassettes may additionally contain 5′ leader sequences in the expression cassette construct. Such leader sequences can act to enhance translation.

It is to be understood that as used herein the term “transgenic” includes any cell, cell line, callus, tissue, plant part, or plant, the genotype of which has been altered by the presence of a heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

A transgenic “event” is produced by transformation of plant cells with a heterologous DNA construct(s), including a nucleic acid expression cassette that comprises a transgene of interest, the regeneration of a population of plants resulting from the insertion of the transgene into the genome of the plant, and selection of a particular plant characterized by insertion into a particular genome location. An event is characterized phenotypically by the expression of the transgene. At the genetic level, an event is part of the genetic makeup of a plant. The term “event” also refers to progeny produced by a sexual outcross between the transformant and another variety that include the heterologous DNA. Even after repeated back-crossing to a recurrent parent, the inserted DNA and flanking DNA from the transformed parent is present in the progeny of the cross at the same chromosomal location. The term “event” also refers to DNA from the original transformant comprising the inserted DNA and flanking sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA.

An insect resistant DP-032218-9 corn plant can be bred by first sexually crossing a first parental corn plant consisting of a corn plant grown from the transgenic DP-032218-9 corn plant and progeny thereof derived from transformation with the expression cassettes of the embodiments of the present disclosure that confers insect resistance, and a second parental corn plant that lacks insect resistance, thereby producing a plurality of first progeny plants; and then selecting a first progeny plant that is resistant to insects; and selfing the first progeny plant, thereby producing a plurality of second progeny plants; and then selecting from the second progeny plants an insect resistant plant. These steps can further include the back-crossing of the first insect resistant progeny plant or the second insect resistant progeny plant to the second parental corn plant or a third parental corn plant, thereby producing a corn plant that is resistant to insects.

As used herein, the term “plant” includes reference to whole plants, plant organs (e.g., leaves, stems, roots, etc.), seeds, plant cells, and progeny of same. Parts of transgenic plants understood to be within the scope of the disclosure comprise, for example, plant cells, protoplasts, tissues, callus, embryos as well as flowers, stems, fruits, leaves, and roots originating in transgenic plants or their progeny previously transformed with a DNA molecule of the disclosure and therefore consisting at least in part of transgenic cells, are also an embodiment of the present disclosure.

As used herein, the term “plant cell” includes, without limitation, seeds, suspension cultures, embryos, meristematic regions, callus tissue, leaves, roots, shoots, gametophytes, sporophytes, pollen, and microspores. The class of plants that can be used in the methods of the disclosure is generally as broad as the class of higher plants amenable to transformation techniques, including both monocotyledonous and dicotyledonous plants.

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include Agrobacterium-mediated transformation (De Blaere et al. (1987) Meth. Enzymol. 143:277) and particle-accelerated or “gene gun” transformation technology (Klein et al. (1987) Nature (London) 327:70-73; U.S. Pat. No. 4,945,050, incorporated herein by reference). Additional transformation methods are disclosed below.

Thus, isolated polynucleotides of the disclosure can be incorporated into recombinant constructs, typically DNA constructs, which are capable of introduction into and replication in a host cell. Such a construct can be a vector that includes a replication system and sequences that are capable of transcription and translation of a polypeptide-encoding sequence in a given host cell. A number of vectors suitable for stable transfection of plant cells or for the establishment of transgenic plants have been described in, e.g., Pouwels et al., (1985; Supp. 1987) Cloning Vectors: A Laboratory Manual, Weissbach and Weissbach (1989) Methods for Plant Molecular Biology, (Academic Press, New York); and Flevin et al., (1990) Plant Molecular Biology Manual, (Kluwer Academic Publishers). Typically, plant expression vectors include, for example, one or more cloned plant genes under the transcriptional control of 5′ and 3′ regulatory sequences and a dominant selectable marker. Such plant expression vectors also can contain, without limitation: a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

It is also to be understood that two different transgenic plants can also be crossed to produce progeny that contain two independently segregating added, exogenous genes. Selfing of appropriate progeny can produce plants that are homozygous for both added, exogenous genes. Back-crossing to a parental plant and out-crossing with a non-transgenic plant are also contemplated, as is vegetative propagation. Descriptions of other breeding methods that are commonly used for different traits and crops can be found in one of several references, e.g., Fehr, in Breeding Methods for Cultivar Development, Wilcos J. ed., American Society of Agronomy, Madison Wis. (1987).

### Seed Treatments

In one embodiment, seeds comprising event DP-032218-9 may be combined with a seed treatment formulation or compound.

The formula can be applied by such methods as drenching the growing medium including the seed with a solution or dispersion, mixing with growing medium and planting the seed in the treated growing medium, or various forms of seed treatments whereby the formulation is applied to the seed before it is planted.

In these methods the seed treatment will generally be used as a formulation or compound with an agriculturally suitable carrier comprising at least one of a liquid diluent, a solid diluent or a surfactant. A wide variety of formulations are suitable for this disclosure, the most suitable types of formulations depend upon the method of application.

Depending on the method of application, useful formulations include, without limitation: liquids such as solutions (including emulsifiable concentrates), suspensions, emulsions (including microemulsions and/or suspoemulsions) and the like which optionally can be thickened into gels.

Useful formulations further include, but are not limited to: solids such as dusts, powders, granules, pellets, tablets, films, and the like which can be water-dispersible (“wettable”) or water-soluble. Active ingredient can be microencapsulated and further formed into a suspension or solid formulation; alternatively the entire formulation of active ingredient can be encapsulated (or “overcoated”). Encapsulation can control or delay release of the active ingredient. Sprayable formulations can be extended in suitable media and used at spray volumes from about one to several hundred liters per hectare.

The disclosure includes a seed contacted with a composition comprising a biologically effective amount of a seed treatment compound and an effective amount of at least one other biologically active compound or agent. The compositions used for treating seeds (or plant grown therefrom) according to this disclosure can also comprise an effective amount of one or more other biologically active compounds or agents. Suitable additional compounds or agents include, but are not limited to: insecticides, fungicides, nematocides, bactericides, acaricides, growth regulators such as rooting stimulants, chemosterilants, semiochemicals, repellents, attractants, pheromones, feeding stimulants, other biologically active compounds or entomopathogenic, viruses, bacteria or fungi to form a multi-component pesticide giving an even broader spectrum of agricultural utility. Examples of such biologically active compounds or agents with which compounds of this disclosure can be formulated are: insecticides such as abamectin, acephate, acetamiprid, amidoflumet (S-1955), avermectin, azadirachtin, azinphos-methyl, bifenthrin, binfenazate, buprofezin, carbofuran, chlorfenapyr, chlorfluazuron, chlorpyrifos, chlorpyrifos-methyl, chromafenozide, clothianidin, cyfluthrin, beta-cyfluthrin, cyhalothrin, lambda-cyhalothrin, cypermethrin, cyromazine, deltamethrin, diafenthiuron, diazinon, diflubenzuron, dimethoate, diofenolan, emamectin, endosulfan, esfenvalerate, ethiprole, fenothicarb, fenoxycarb, fenpropathrin, fenproximate, fenvalerate, fipronil, flonicamid, flucythrinate, tau-fluvalinate, flufenerim (UR-50701), flufenoxuron, fonophos, halofenozide, hexaflumuron, imidacloprid, indoxacarb, isofenphos, lufenuron, malathion, metaldehyde, methamidophos, methidathion, methomyl, methoprene, methoxychlor, monocrotophos, methoxyfenozide, nithiazin, novaluron, noviflumuron (XDE-007), oxamyl, parathion, parathion-methyl, permethrin, phorate, phosalone, phosmet, phosphamidon, pirimicarb, profenofos, pymetrozine, pyridalyl, pyriproxyfen, rotenone, spinosad, spiromesifin (BSN 2060), sulprofos, tebufenozide, teflubenzuron, tefluthrin, terbufos, tetrachlorvinphos, thiacloprid, thiamethoxam, thiodicarb, thiosultap-sodium, tralomethrin, trichlorfon and triflumuron; fungicides such as acibenzolar, azoxystrobin, benomyl, blasticidin-S, Bordeaux mixture (tribasic copper sulfate), bromuconazole, carpropamid, captafol, captan, carbendazim, chloroneb, chlorothalonil, copper oxychloride, copper salts, cyflufenamid, cymoxanil, cyproconazole, cyprodinil, (S)-3,5-dichloro-N-(3-chloro-1-ethyl-1-methyl-2-oxopropyl)-4-methylbenzamide (RH 7281), diclocymet (S-2900), diclomezine, dicloran, difenoconazole, (S)-3,5-dihydro-5-methyl-2-(methylthio)-5-phenyl-3-(phenylamino)-4H-imidazol-4-one (RP 407213), dimethomorph, dimoxystrobin, diniconazole, diniconazole-M, dodine, edifenphos, epoxiconazole, famoxadone, fenamidone, fenarimol, fenbuconazole, fencaramid (SZX0722), fenpiclonil, fenpropidin, fenpropimorph, fentin acetate, fentin hydroxide, fluazinam, fludioxonil, flumetover (RPA 403397), flumorf/flumorlin (SYP-L190), fluoxastrobin (HEC 5725), fluquinconazole, flusilazole, flutolanil, flutriafol, folpet, fosetyl-aluminum, furalaxyl, furametapyr (S-82658), hexaconazole, ipconazole, iprobenfos, iprodione, isoprothiolane, kasugamycin, kresoxim-methyl, mancozeb, maneb, mefenoxam, mepronil, metalaxyl, metconazole, metominostrobin/fenominostrobin (SSF-126), metrafenone (AC 375839), myclobutanil, neo-asozin (ferric methanearsonate), nicobifen (BAS 510), orysastrobin, oxadixyl, penconazole, pencycuron, probenazole, prochloraz, propamocarb, propiconazole, proquinazid (DPX-KQ926), prothioconazole (JAU 6476), pyrifenox, pyraclostrobin, pyrimethanil, pyroquilon, quinoxyfen, spiroxamine, sulfur, tebuconazole, tetraconazole, thiabendazole, thifluzamide, thiophanate-methyl, thiram, tiadinil, triadimefon, triadimenol, tricyclazole, trifloxystrobin, triticonazole, validamycin and vinclozolin; nematocides such as aldicarb, oxamyl and fenamiphos; bactericides such as streptomycin; and acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad.

Examples of entomopathic viruses include, but are not limited to, species classified as baculoviruses, ascoviruses, iridoviruses, parvoviruses, polydnavirusespoxviruses, reoviruses and tetraviruses. Examples also include entomopathoic viruses that have been genetically modified with additional beneficial properties (Gramkow, A. W. et al., 2010 Virology Journal 7, art. no. 143; Shim, et al., 2009 Journal of Asia-pacific Entomology 12(4): 217-220).

Examples of entomopathic bacteria include, but are not limited to, species within the genera Bacillus (including B. cereus, B. popilliae, B. sphaericus and B. thuringiensis), Enterococcus, Fischerella, Lysinibacillus, Photorhabdus, Pseudomonas, Saccharopolyspora, Streptomyces, Xenorhabdus and Yersinia (see, for example, Barry, C., 2012 Journal of Invertebrate Pathology 109(1): 1-10; Sanchis, V., 2011 Agronomy for Sustainable Development 31(1): 217-231; Mason, K. L., et al., 2011 mBio 2(3): e00065-11; Muratoglu, H., et al., 2011 Turkish Journal of Biology 35(3): 275-282; Hincliffe, S. J., et al., 2010 The Open Toxinology Journal 3: 101-118; Kirst, H. A., 2010 Journal of Antibiotics 63(3): 101-111; Shu, C. and Zhang, J., 2009 Recent Patents on DNA and Gene Sequences 3(1): 26-28; Becher, P. J., et al., 2007 Phytochemistry68(19): 2493-2497; Dodd, S. J., et al., 2006 Applied and Environmental Microbiology 72(10): 6584-6592; Zhang, J., et al. 1997 Journal of Bacteriology 179(13): 4336-4341.

Examples of entomopathic fungi include, but are not limited to species within the genera Beauveria (e.g., B. bassiana), Cordyceps, Lecanicillium, Metarhizium (e.g., M. anisopliae), Nomuraea and Paecilomyces (US20120128648, WO2011099022, US20110038839, U.S. Pat. No. 7,416,880, U.S. Pat. No. 6,660,290; Tang, L.-C. and Hou, R. F., 1998 Entomolgia Experimentalis et Applicata 88(1): 25-30) Examples of entomopathic nematodes include, but are not limited to, species within the genera Heterorhabditis and Steinernema (U.S. Pat. No. 6,184,434).

A general reference for these agricultural protectants is The Pesticide Manual, 12th Edition, C. D. S. Tomlin, Ed., British Crop Protection Council, Farnham, Surrey, U. K., 2000, L. G. Copping, ed., 2009 The Manual of Biocontrol Agents: A World Compendium (4^{th} ed., CABI Publishing); and Dev, S. and Koul, O., 1997 Insecticides of Natural Origin, CRC Press; EPA Biopesticides web publication, last viewed on May 25, 2012).

### Insect Resistance Management and Event Stacking

In one embodiment, the efficacy of event DP-032218-9 against target pests is increased and the development of resistant insects is reduced by use of a non-transgenic “refuge”—a section of non-insecticidal corn or other crop.

The United States Environmental Protection Agency publishes the requirements for use with transgenic crops producing a single Bt protein active against target pests, see: (epa.gov/oppbppdl/biopesticides/pips/bt_corn_refuge_2006.htm, which can be accessed using the www prefix). In addition, the National Corn Growers Association, on their website: (ncga.com/insect-resistance-management-fact-sheet-bt-corn, which can be accessed using the www prefix) also provides similar guidance regarding refuge requirements.

Expression in a plant of two or more insecticidal compositions toxic to the same insect species, each insecticide being expressed at levels high enough to effectively delay the onset of resistance, would be another way to achieve control of the development of resistance. Roush et al., for example, outlines two-toxin strategies, also called “pyramiding” or “stacking,” for management of insecticidal transgenic crops. (The Royal Society. Phil. Trans. R. Soc. Lond. B. (1998) 353, 1777-1786). Stacking or pyramiding of two different proteins each effective against the target pests and with little or no cross-resistance can allow for use of a smaller refuge. The U.S. Environmental Protection Agency requires significantly less (generally 5%) structured refuge of non-Bt corn be planted than for single trait products (generally 20%). There are various ways of providing the IRM effects of a refuge, including various geometric planting patterns in the fields and in-bag seed mixtures, as discussed further by Roush et al.

In certain embodiments the event of the present disclosure can be “stacked”, or combined, with any combination of polynucleotide sequences of interest in order to create plants with a desired trait. A trait, as used herein, refers to the phenotype derived from a particular sequence or groups of sequences. For example, the event of the present disclosure, may be stacked with any other polynucleotides encoding polypeptides of interest.

In one embodiment, maize event DP-032218-9 can be stacked with other genes conferring pesticidal and/or insecticidal activity, such as other Bacillus thuringiensis toxic proteins (described in U.S. Pat. Nos. 5,366,892; 5,747,450; 5,737,514; 5,723,756; 5,593,881; and Geiser et al. (1986) Gene 48:109), lectins (Van Damme et al. (1994) Plant Mol. Biol. 24:825, pentin (described in U.S. Pat. No. 5,981,722), and the like.

The combinations generated can also include multiple copies of any one of the polynucleotides of interest. The polynucleotides of the present disclosure can also be stacked with any other gene or combination of genes to produce plants with a variety of desired trait combinations including, but not limited to, balanced amino acids (e.g., hordothionins (U.S. Pat. Nos. 5,990,389; 5,885,801; 5,885,802; and 5,703,409); barley high lysine (Williamson et al. (1987) Eur. J. Biochem. 165:99-106; and WO 98/20122) and high methionine proteins (Pedersen et al. (1986) J. Biol. Chem. 261:6279; Kirihara et al. (1988) Gene 71:359 and Musumura et al. (1989) Plant Mol. Biol. 12:123); and thioredoxins (Sewalt et al., U.S. Pat. No. 7,009,087).

The polynucleotides of the present disclosure can also be stacked with traits desirable for disease or herbicide resistance (e.g., fumonisin detoxification genes (U.S. Pat. No. 5,792,931); avirulence and disease resistance genes (Jones et al. (1994) Science 266:789; Martin et al. (1993) Science 262:1432; Mindrinos et al. (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al. (1988) J. Bacteriol. 170:5837-5847) facilitate expression of polyhydroxyalkanoates (PHAs)). One could also combine the polynucleotides of the present disclosure with polynucleotides providing agronomic traits such as male sterility (e.g., see U.S. Pat. No. 5,583,210), stalk strength, flowering time, or transformation technology traits such as cell cycle regulation or gene targeting (e.g., WO 99/61619, WO 00/17364, and WO 99/25821).

Non-limiting examples of events that may be combined with the event of the present disclosure are shown in Table 1.

TABLE 1

| Event | Company | Description |
|---|---|---|
| 176 | Syngenta Seeds, Inc. | Insect-resistant maize produced by |
| | | inserting the cry1Ab gene from Bacillus |
| | | thuringiensis subsp. kurstaki. The genetic |
| | | modification affords resistance to attack by |
| | | the European corn borer (ECB). |
| 3751IR | Pioneer Hi-Bred | Selection of somaclonal variants by culture |
| | International Inc. | of embryos on imidazolinone containing |
| | | media. |
| 676, 678, 680 | Pioneer Hi-Bred | Male-sterile and glufosinate ammonium |
| | International Inc. | herbicide tolerant maize produced by |
| | | inserting genes encoding DNA adenine |
| | | methylase and phosphinothricin |
| | | acetyltransferase (PAT) from Escherichia |
| | | coli and Streptomyces viridochromogenes, |
| | | respectively. |
| B16 (DLL25) | Dekalb Genetics | Glufosinate ammonium herbicide tolerant |
| | Corporation | maize produced by inserting the gene |
| | | encoding phosphinothricin acetyltransferase |
| | | (PAT) from Streptomyces hygroscopicus. |
| BT11 (X4334CBR, | Syngenta Seeds, Inc. | Insect-resistant and herbicide tolerant |
| X4734CBR) | | maize produced by inserting the cry1Ab |
| | | gene from Bacillus thuringiensis subsp. |
| | | kurstaki, and the phosphinothricin N- |
| | | acetyltransferase (PAT) encoding gene |
| | | from S. viridochromogenes. |
| BT11 × GA21 | Syngenta Seeds, Inc. | Stacked insect resistant and herbicide |
| | | tolerant maize produced by conventional |
| | | cross breeding of parental lines BT11 |
| | | (OECD unique identifier: SYN-BTO11-1) |
| | | and GA21 (OECD unique identifier: MON- |
| | | OOO21-9). |
| BT11 × MIR162 | Syngenta Seeds, Inc. | Stacked insect resistant and herbicide |
| | | tolerant maize produced by conventional |
| | | cross breeding of parental lines BT11 |
| | | (OECD unique identifier: SYN-BTO11-1) |
| | | and MIR162 (OECD unique identifier: SYN- |
| | | IR162-4). Resistance to the European Corn |
| | | Borer and tolerance to the herbicide |
| | | glufosinate ammonium (Liberty) is derived |
| | | from BT11, which contains the cry1Ab gene |
| | | from Bacillus thuringiensis subsp. kurstaki, |
| | | and the phosphinothricin N- |
| | | acetyltransferase (PAT) encoding gene |
| | | from S. viridochromogenes. Resistance to |
| | | other lepidopteran pests, including H. zea, |
| | | S. frugiperda, A. ipsilon, and S. albicosta, is |
| | | derived from MIR162, which contains the |
| | | vip3Aa gene from Bacillus thuringiensis |
| | | strain AB88. |
| BT11 × MIR162 × | Syngenta Seeds, Inc. | Bacillus thuringiensis Cry1Ab delta- |
| MIR604 | | endotoxin protein and the genetic material |
| | | necessary for its production (via elements |
| | | of vector pZO1502) in Event Bt11 corn |
| | | (OECD Unique Identifier: SYN-BTO11-1) × |
| | | Bacillus thuringiensis Vip3Aa20 insecticidal |
| | | protein and the genetic material necessary |
| | | for its production (via elements of vector |
| | | pNOV1300) in Event MIR162 maize (OECD |
| | | Unique Identifier: SYN-IR162-4) × modified |
| | | Cry3A protein and the genetic material |
| | | necessary for its production (via elements |
| | | of vector pZM26) in Event MIR604 corn |
| | | (OECD Unique Identifier: SYN-IR6O4-5). |
| BT11 × MIR162 × | Syngenta Seeds, Inc. | Resistance to coleopteran pests, |
| MIR604 × GA21 | | particularly corn rootworm pests (Diabrotica |
| | | spp.) and several lepidopteran pests of |
| | | corn, including European corn borer (ECB, |
| | | Ostrinia nubilalis), corn earworm (CEW, |
| | | Helicoverpa zea), fall army worm (FAW, |
| | | Spodoptera frugiperda), and black cutworm |
| | | (BCW, Agrotis ipsilon); tolerance to |
| | | glyphosate and glufosinate-ammonium |
| | | containing herbicides. |
| BT11 × MIR604 | Syngenta Seeds, Inc. | Stacked insect resistant and herbicide |
| | | tolerant maize produced by conventional |
| | | cross breeding of parental lines BT11 |
| | | (OECD unique identifier: SYN-BTO11-1) |
| | | and MIR604 (OECD unique identifier: SYN- |
| | | IR6O5-5). Resistance to the European Corn |
| | | Borer and tolerance to the herbicide |
| | | glufosinate ammonium (Liberty) is derived |
| | | from BT11, which contains the cry1Ab gene |
| | | from Bacillus thuringiensis subsp. kurstaki, |
| | | and the phosphinothricin N- |
| | | acetyltransferase (PAT) encoding gene |
| | | from S. viridochromogenes. Corn rootworm- |
| | | resistance is derived from MIR604 which |
| | | contains the mcry3A gene from Bacillus |
| | | thuringiensis. |
| BT11 × MIR604 × | Syngenta Seeds, Inc. | Stacked insect resistant and herbicide |
| GA21 | | tolerant maize produced by conventional |
| | | cross breeding of parental lines BT11 |
| | | (OECD unique identifier: SYN-BTO11-1), |
| | | MIR604 (OECD unique identifier: SYN- |
| | | IR6O5-5) and GA21 (OECD unique |
| | | identifier: MON-OOO21-9). Resistance to |
| | | the European Corn Borer and tolerance to |
| | | the herbicide glufosinate ammonium |
| | | (Liberty) is derived from BT11, which |
| | | contains the cry1Ab gene from Bacillus |
| | | thuringiensis subsp. kurstaki, and the |
| | | phosphinothricin N-acetyltransferase (PAT) |
| | | encoding gene from S. viridochromogenes. |
| | | Corn rootworm-resistance is derived from |
| | | MIR604 which contains the mcry3A gene |
| | | from Bacillus thuringiensis. Tolerance to |
| | | glyphosate herbicide is derived from GA21 |
| | | which contains a a modified EPSPS gene |
| | | from maize. |
| CBH-351 | Aventis CropScience | Insect-resistant and glufosinate ammonium |
| | | herbicide tolerant maize developed by |
| | | inserting genes encoding Cry9C protein |
| | | from Bacillus thuringiensis subsp tolworthi |
| | | and phosphinothricin acetyltransferase |
| | | (PAT) from Streptomyces hygroscopicus. |
| DAS-06275-8 | DOW AgroSciences | Lepidopteran insect resistant and |
| | LLC | glufosinate ammonium herbicide-tolerant |
| | | maize variety produced by inserting the |
| | | cry1F gene from Bacillus thuringiensis var |
| | | aizawai and the phosphinothricin |
| | | acetyltransferase (PAT) from Streptomyces |
| | | hygroscopicus. |
| DAS-59122-7 | DOW AgroSciences | Corn rootworm-resistant maize produced by |
| | LLC and Pioneer Hi- | inserting the cry34Ab1 and cry35Ab1 genes |
| | Bred International Inc. | from Bacillus thuringiensis strain PS149B1. |
| | | The PAT encoding gene from Streptomyces |
| | | viridochromogenes was introduced as a |
| | | selectable marker. |
| DAS-59122-7 × | DOW AgroSciences | Stacked insect resistant and herbicide |
| NK603 | LLC and Pioneer Hi- | tolerant maize produced by conventional |
| | Bred International Inc. | cross breeding of parental lines DAS- |
| | | 59122-7 (OECD unique identifier: DAS- |
| | | 59122-7) with NK603 (OECD unique |
| | | identifier: MON-OO6O3-6). Corn rootworm- |
| | | resistance is derived from DAS-59122-7 |
| | | which contains the cry34Ab1 and cry35Ab1 |
| | | genes from Bacillus thuringiensis strain |
| | | PS149B1. Tolerance to glyphosate |
| | | herbicide is derived from NK603. |
| DAS-59122-7 × | DOW AgroSciences | Stacked insect resistant and herbicide |
| TC1507 × NK603 | LLC and Pioneer Hi- | tolerant maize produced by conventional |
| | Bred International Inc. | cross breeding of parental lines DAS- |
| | | 59122-7 (OECD unique identifier: DAS- |
| | | 59122-7) and TC1507 (OECD unique |
| | | identifier: DAS-O15O7-1) with NK603 |
| | | (OECD unique identifier: MON-OO6O3-6). |
| | | Corn rootworm-resistance is derived from |
| | | DAS-59122-7 which contains the cry34Ab1 |
| | | and cry35Ab1 genes from Bacillus |
| | | thuringiensis strain PS149B1. Lepidopteran |
| | | resistance and tolerance to glufosinate |
| | | ammonium herbicide is derived from |
| | | TC1507. Tolerance to glyphosate herbicide |
| | | is derived from NK603. |
| DBT418 | Dekalb Genetics | Insect-resistant and glufosinate ammonium |
| | Corporation | herbicide tolerant maize developed by |
| | | inserting genes encoding Cry1AC protein |
| | | from Bacillus thuringiensis subsp kurstaki |
| | | and phosphinothricin acetyltransferase |
| | | (PAT) from Streptomyces hygroscopicus |
| DK404SR | BASF Inc. | Somaclonal variants with a modified acetyl- |
| | | CoA-carboxylase (ACCase) were selected |
| | | by culture of embryos on sethoxydim |
| | | enriched medium. |
| Event 3272 | Syngenta Seeds, Inc. | Maize line expressing a heat stable alpha- |
| | | amylase gene amy797E for use in the dry- |
| | | grind ethanol process. The |
| | | phosphomannose isomerase gene from |
| | | E. coli was used as a selectable marker. |
| Event 98140 | Pioneer Hi-Bred | Maize event expressing tolerance to |
| | International Inc. | glyphosate herbicide, via expression of a |
| | | modified bacterial glyphosate N- |
| | | acetlytransferase, and ALS-inhibiting |
| | | herbicides, vial expression of a modified |
| | | form of the maize acetolactate synthase |
| | | enzyme. |
| EXP1910IT | Syngenta Seeds, Inc. | Tolerance to the imidazolinone herbicide, |
| | (formerly Zeneca | imazethapyr, induced by chemical |
| | Seeds) | mutagenesis of the acetolactate synthase |
| | | (ALS) enzyme using ethyl |
| | | methanesulfonate (EMS). |
| GA21 | Syngenta Seeds, Inc. | Introduction, by particle bombardment, of a |
| | (formerly Zeneca | modified 5-enolpyruvyl shikimate-3- |
| | Seeds) | phosphate synthase (EPSPS), an enzyme |
| | | involved in the shikimate biochemical |
| | | pathway for the production of the aromatic |
| | | amino acids. |
| GA21 × MON810 | Monsanto Company | Stacked insect resistant and herbicide |
| | | tolerant corn hybrid derived from |
| | | conventional cross-breeding of the parental |
| | | lines GA21 (OECD identifier: MON-OOO21- |
| | | 9) and MON810 (OECD identifier: MON- |
| | | OO81O-6). |
| IT | Pioneer Hi-Bred | Tolerance to the imidazolinone herbicide, |
| | International Inc. | imazethapyr, was obtained by in vitro |
| | | selection of somaclonal variants. |
| LY038 | Monsanto Company | Altered amino acid composition, specifically |
| | | elevated levels of lysine, through the |
| | | introduction of the cordapA gene, derived |
| | | from Corynebacterium glutamicum, |
| | | encoding the enzyme dihydrodipicolinate |
| | | synthase (cDHDPS). |
| MIR162 | Syngenta Seeds, Inc. | Insect-resistant maize event expressing a |
| | | Vip3A protein from Bacillus thuringiensis |
| | | and the Escherichia coli PMI selectable |
| | | marker |
| MIR604 | Syngenta Seeds, Inc. | Corn rootworm resistant maize produced by |
| | | transformation with a modified cry3A gene. |
| | | The phosphomannose isomerase gene |
| | | from E. coli was used as a selectable |
| | | marker. |
| MIR604 × GA21 | Syngenta Seeds, Inc. | Stacked insect resistant and herbicide |
| | | tolerant maize produced by conventional |
| | | cross breeding of parental lines MIR604 |
| | | (OECD unique identifier: SYN-IR6O5-5) |
| | | and GA21 (OECD unique identifier: MON- |
| | | OOO21-9). Corn rootworm-resistance is |
| | | derived from MIR604 which contains the |
| | | mcry3A gene from Bacillus thuringiensis. |
| | | Tolerance to glyphosate herbicide is |
| | | derived from GA21. |
| MON80100 | Monsanto Company | Insect-resistant maize produced by |
| | | inserting the cry1Ab gene from Bacillus |
| | | thuringiensis subsp. kurstaki. The genetic |
| | | modification affords resistance to attack by |
| | | the European corn borer (ECB). |
| MON802 | Monsanto Company | Insect-resistant and glyphosate herbicide |
| | | tolerant maize produced by inserting the |
| | | genes encoding the Cry1Ab protein from |
| | | Bacillus thuringiensis and the 5- |
| | | enolpyruvylshikimate-3-phosphate synthase |
| | | (EPSPS) from A. tumefaciens strain CP4. |
| MON809 | Pioneer Hi-Bred | Resistance to European corn borer |
| | International Inc. | (Ostrinia nubilalis) by introduction of a |
| | | synthetic cry1Ab gene. Glyphosate |
| | | resistance via introduction of the bacterial |
| | | version of a plant enzyme, 5-enolpyruvyl |
| | | shikimate-3-phosphate synthase (EPSPS). |
| MON810 | Monsanto Company | Insect-resistant maize produced by |
| | | inserting a truncated form of the cry1Ab |
| | | gene from Bacillus thuringiensis subsp. |
| | | kurstaki HD-1. The genetic modification |
| | | affords resistance to attack by the |
| | | European corn borer (ECB). |
| MON810 × LY038 | Monsanto Company | Stacked insect resistant and enhanced |
| | | lysine content maize derived from |
| | | conventional cross-breeding of the parental |
| | | lines MON810 (OECD identifier: MON- |
| | | OO81O-6) and LY038 (OECD identifier: |
| | | REN-OOO38-3). |
| MON810 × | Monsanto Company | Stacked insect resistant and glyphosate |
| MON88017 | | tolerant maize derived from conventional |
| | | cross-breeding of the parental lines |
| | | MON810 (OECD identifier: MON-OO81O-6) |
| | | and MON88017 (OECD identifier: MON- |
| | | 88O17-3). European corn borer (ECB) |
| | | resistance is derived from a truncated form |
| | | of the cry1Ab gene from Bacillus |
| | | thuringiensis subsp. kurstaki HD-1 present |
| | | in MON810. Corn rootworm resistance is |
| | | derived from the cry3Bb1 gene from |
| | | Bacillus thuringiensis subspecies |
| | | kumamotoensis strain EG4691 present in |
| | | MON88017. Glyphosate tolerance is |
| | | derived from a 5-enolpyruvylshikimate-3- |
| | | phosphate synthase (EPSPS) encoding |
| | | gene from Agrobacterium tumefaciens |
| | | strain CP4 present in MON88017. |
| MON832 | Monsanto Company | Introduction, by particle bombardment, of |
| | | glyphosate oxidase (GOX) and a modified |
| | | 5-enolpyruvyl shikimate-3-phosphate |
| | | synthase (EPSPS), an enzyme involved in |
| | | the shikimate biochemical pathway for the |
| | | production of the aromatic amino acids. |
| MON863 | Monsanto Company | Corn root worm resistant maize produced |
| | | by inserting the cry3Bb1 gene from Bacillus |
| | | thuringiensis subsp. kumamotoensis. |
| MON863 × MON810 | Monsanto Company | Stacked insect resistant corn hybrid derived |
| | | from conventional cross-breeding of the |
| | | parental lines MON863 (OECD identifier: |
| | | MON-OO863-5) and MON810 (OECD |
| | | identifier: MON-OO81O-6) |
| MON863 × MON810 × | Monsanto Company | Stacked insect resistant and herbicide |
| NK603 | | tolerant corn hybrid derived from |
| | | conventional cross-breeding of the stacked |
| | | hybrid MON-OO863-5 × MON-OO81O-6 |
| | | and NK603 (OECD identifier: MON-OO6O3- |
| | | 6). |
| MON863 × NK603 | Monsanto Company | Stacked insect resistant and herbicide |
| | | tolerant corn hybrid derived from |
| | | conventional cross-breeding of the parental |
| | | lines MON863 (OECD identifier: MON- |
| | | OO863-5) and NK603 (OECD identifier: |
| | | MON-OO6O3-6). |
| MON87460 | Monsanto Company | MON 87460 was developed to provide |
| | | reduced yield loss under water-limited |
| | | conditions compared to conventional maize. |
| | | Efficacy in MON 87460 is derived by |
| | | expression of the inserted Bacillus subtilis |
| | | cold shock protein B (CspB). |
| MON88017 | Monsanto Company | Corn rootworm-resistant maize produced by |
| | | inserting the cry3Bb1 gene from Bacillus |
| | | thuringiensis subspecies kumamotoensis |
| | | strain EG4691. Glyphosate tolerance |
| | | derived by inserting a 5- |
| | | enolpyruvylshikimate-3-phosphate synthase |
| | | (EPSPS) encoding gene from |
| | | Agrobacterium tumefaciens strain CP4. |
| MON89034 | Monsanto Company | Maize event expressing two different |
| | | insecticidal proteins from Bacillus |
| | | thuringiensis providing resistance to |
| | | number of lepidopteran pests. |
| MON89034 × | Monsanto Company | Stacked insect resistant and glyphosate |
| MON88017 | | tolerant maize derived from conventional |
| | | cross-breeding of the parental lines |
| | | MON89034 (OECD identifier: MON-89O34- |
| | | 3) and MON88017 (OECD identifier: MON- |
| | | 88O17-3). Resistance to Lepidopteran |
| | | insects is derived from two cry genes |
| | | present in MON89043. Corn rootworm |
| | | resistance is derived from a single cry |
| | | genes and glyphosate tolerance is derived |
| | | from the 5-enolpyruvylshikimate-3- |
| | | phosphate synthase (EPSPS) encoding |
| | | gene from Agrobacterium tumefaciens |
| | | present in MON88017. |
| MON89034 × NK603 | Monsanto Company | Stacked insect resistant and herbicide |
| | | tolerant maize produced by conventional |
| | | cross breeding of parental lines MON89034 |
| | | (OECD identifier: MON-89O34-3) with |
| | | NK603 (OECD unique identifier: MON- |
| | | OO6O3-6). Resistance to Lepidopteran |
| | | insects is derived from two cry genes |
| | | present in MON89043. Tolerance to |
| | | glyphosate herbicide is derived from |
| | | NK603. |
| MON89034 × | Monsanto Company | Stacked insect resistant and herbicide |
| TC1507 × | and Mycogen Seeds | tolerant maize produced by conventional |
| MON88017 × DAS- | c/o Dow | cross breeding of parental lines: |
| 59122-7 | AgroSciences LLC | MON89034, TC1507, MON88017, and |
| | | DAS-59122. Resistance to the above- |
| | | ground and below-ground insect pests and |
| | | tolerance to glyphosate and glufosinate- |
| | | ammonium containing herbicides. |
| MS3 | Bayer CropScience | Male sterility caused by expression of the |
| | (Aventis | barnase ribonuclease gene from Bacillus |
| | CropScience(AgrEvo)) | amyloliquefaciens; PPT resistance was via |
| | | PPT-acetyltransferase (PAT). |
| MS6 | Bayer CropScience | Male sterility caused by expression of the |
| | (Aventis | barnase ribonuclease gene from Bacillus |
| | CropScience(AgrEvo)) | amyloliquefaciens; PPT resistance was via |
| | | PPT-acetyltransferase (PAT). |
| NK603 | Monsanto Company | Introduction, by particle bombardment, of a |
| | | modified 5-enolpyruvyl shikimate-3- |
| | | phosphate synthase (EPSPS), an enzyme |
| | | involved in the shikimate biochemical |
| | | pathway for the production of the aromatic |
| | | amino acids. |
| NK603 × MON810 | Monsanto Company | Stacked insect resistant and herbicide |
| | | tolerant corn hybrid derived from |
| | | conventional cross-breeding of the parental |
| | | lines NK603 (OECD identifier: MON- |
| | | OO6O3-6) and MON810 (OECD identifier: |
| | | MON-OO81O-6). |
| NK603 × T25 | Monsanto Company | Stacked glufosinate ammonium and |
| | | glyphosate herbicide tolerant maize hybrid |
| | | derived from conventional cross-breeding of |
| | | the parental lines NK603 (OECD identifier: |
| | | MON-OO6O3-6) and T25 (OECD identifier: |
| | | ACS-ZM003-2). |
| T14, T25 | Bayer CropScience | Glufosinate herbicide tolerant maize |
| | (Aventis | produced by inserting the phosphinothricin |
| | CropScience(AgrEvo)) | N-acetyltransferase (PAT) encoding gene |
| | | from the aerobic actinomycete |
| | | Streptomyces viridochromogenes. |
| T25 × MON810 | Bayer CropScience | Stacked insect resistant and herbicide |
| | (Aventis | tolerant corn hybrid derived from |
| | CropScience(AgrEvo)) | conventional cross-breeding of the parental |
| | | lines T25 (OECD identifier: ACS-ZMOO3-2) |
| | | and MON810 (OECD identifier: MON- |
| | | OO81O-6). |
| TC1507 | Mycogen (c/o Dow | Insect-resistant and glufosinate ammonium |
| | AgroSciences); | herbicide tolerant maize produced by |
| | Pioneer (c/o DuPont) | inserting the cry1F gene from Bacillus |
| | | thuringiensis var. aizawai and the |
| | | phosphinothricin N-acetyltransferase |
| | | encoding gene from Streptomyces |
| | | viridochromogenes. |
| TC1507 × DAS- | DOW AgroSciences | Stacked insect resistant and herbicide |
| 59122-7 | LLC and Pioneer Hi- | tolerant maize produced by conventional |
| | Bred International Inc. | cross breeding of parental lines TC1507 |
| | | (OECD unique identifier: DAS-O15O7-1) |
| | | with DAS-59122-7 (OECD unique identifier: |
| | | DAS-59122-7). Resistance to lepidopteran |
| | | insects is derived from TC1507 due the |
| | | presence of the cry1F gene from Bacillus |
| | | thuringiensis var. aizawai. Corn rootworm- |
| | | resistance is derived from DAS-59122-7 |
| | | which contains the cry34Ab1 and cry35Ab1 |
| | | genes from Bacillus thuringiensis strain |
| | | PS149B1. Tolerance to glufosinate |
| | | ammonium herbicide is derived from |
| | | TC1507 from the phosphinothricin N- |
| | | acetyltransferase encoding gene from |
| | | Streptomyces viridochromogenes. |
| TC1507 × NK603 | DOW AgroSciences | Stacked insect resistant and herbicide |
| | LLC | tolerant corn hybrid derived from |
| | | conventional cross-breeding of the parental |
| | | lines 1507 (OECD identifier: DAS-O15O7-1) |
| | | and NK603 (OECD identifier: MON- |
| | | OO6O3-6). |

These stacked combinations can be created by any method including, but not limited to, cross-breeding plants by any conventional or TopCross® methodology, or genetic modification. If the sequences are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. Expression of the sequences can be driven by the same promoter or by different promoters. In certain cases, it may be desirable to introduce a transformation cassette that will suppress the expression of another polynucleotide of interest. This may be combined with any combination of other suppression cassettes or over-expression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853.

In another embodiment, the event of the disclosure can be combined with traits native to certain maize lines that can be identified by a quantitative trait locus (QTL).

The term “quantitative trait locus” or “QTL” refers to a polymorphic genetic locus with at least one allele that correlates with the differential expression of a phenotypic trait in at least one genetic background, e.g., in at least one breeding population or progeny. A QTL can act through a single gene mechanism or by a polygenic mechanism. Examples of QTL traits that may be combined with the event of the disclosure include, but are not limited to: Fusarium resistance (US Pat Pub No: 2010/0269212), Head Smut resistance (US Pat Pub No: 2010/0050291); Colleotrichum resistance (U.S. Pat. No. 8,062,847); and increased oil (U.S. Pat. No. 8,084,208).

In another embodiment, the event of the disclosure can be combined with genes that create a site for site specific DNA integration. This includes the introduction of FRT sites that may be used in the FLP/FRT system and/or Lox sites that may be used in the Cre/Lox system. For example, see Lyznik, et al., Site-Specific Recombination for Genetic Engineering in Plants, Plant Cell Rep (2003) 21:925-932 and WO 99/25821.

A “probe” is an isolated nucleic acid to which is attached a conventional detectable label or reporter molecule, e.g., a radioactive isotope, ligand, chemiluminescent agent, or enzyme. Such a probe is complementary to a strand of a target nucleic acid, in the case of the present disclosure, to a strand of isolated DNA from corn event DP-032218-9 whether from a corn plant or from a sample that includes DNA from the event. Probes according to the present disclosure include not only deoxyribonucleic or ribonucleic acids but also polyamides and other probe materials that bind specifically to a target DNA sequence and can be used to detect the presence of that target DNA sequence.

“Primers” are isolated nucleic acids that are annealed to a complementary target DNA strand by nucleic acid hybridization to form a hybrid between the primer and the target DNA strand, then extended along the target DNA strand by a polymerase, e.g., a DNA polymerase. Primer pairs of the disclosure refer to their use for amplification of a target nucleic acid sequence, e.g., by PCR or other conventional nucleic-acid amplification methods. “PCR” or “polymerase chain reaction” is a technique used for the amplification of specific DNA segments (see, U.S. Pat. Nos. 4,683,195 and 4,800,159; herein incorporated by reference).

Probes and primers are of sufficient nucleotide length to bind to the target DNA sequence specifically in the hybridization conditions or reaction conditions determined by the operator. This length may be of any length that is of sufficient length to be useful in a detection method of choice. Generally, 11 nucleotides or more in length, 18 nucleotides or more, and 22 nucleotides or more, are used. Such probes and primers hybridize specifically to a target sequence under high stringency hybridization conditions. Probes and primers according to embodiments of the present disclosure may have complete DNA sequence similarity of contiguous nucleotides with the target sequence, although probes differing from the target DNA sequence and that retain the ability to hybridize to target DNA sequences may be designed by conventional methods. Probes can be used as primers, but are generally designed to bind to the target DNA or RNA and are not used in an amplification process.

Specific primers can be used to amplify an integration fragment to produce an amplicon that can be used as a “specific probe” for identifying event DP-032218-9 in biological samples. When the probe is hybridized with the nucleic acids of a biological sample under conditions which allow for the binding of the probe to the sample, this binding can be detected and thus allow for an indication of the presence of event DP-032218-9 in the biological sample. Such identification of a bound probe has been described in the art.

In an embodiment of the disclosure the specific probe is a sequence which, under optimized conditions, hybridizes specifically to a region within the 5′ or 3′ flanking region of the event and also comprises a part of the foreign DNA contiguous therewith. The specific probe may comprise a sequence of at least 80%, between 80 and 85%, between 85 and 90%, between 90 and 95%, and between 95 and 100% identical (or complementary) to a specific region of the event.

Methods for preparing and using probes and primers are described, for example, in Sambrook et al., Molecular Cloning: A Laboratory Manual, 2^{nd} ed., vol. 1-3, Cold Spring Harbor Laboratory Press, Cold Spring Harbor, N.Y. 1989 (hereinafter, “Sambrook et al., 1989”); Ausubel et al. eds., Current Protocols in Molecular Biology, Greene Publishing and Wiley-lnterscience, New York, 1995 (with periodic updates) (hereinafter, “Ausubel et al., 1995”); and Innis et al., PCR Protocols: A Guide to Methods and Applications, Academic Press: San Diego, 1990. PCR primer pairs can be derived from a known sequence, for example, by using computer programs intended for that purpose such as the PCR primer analysis tool in Vector NTI version 6 (Informax Inc., Bethesda Md.); PrimerSelect (DNASTAR Inc., Madison, Wis.); and Primer (Version 0.5©, 1991, Whitehead Institute for Biomedical Research, Cambridge, Mass.). Additionally, the sequence can be visually scanned and primers manually identified using guidelines known to one of skill in the art.

Suitable fluorescent dyes include but are not limited to Acridine, AMCA, BODIPY FL-Br2, BODIPY 530/550, BODIPY TMR, BODIPY 558/568, BODIPY 564/570, BODIPY 576/589, BODIPY 581/591, BODIPY TR, BODIPY 630/650*, BODIPY 650/665, Cascade Blue, Cy2, Cy3, Cy3.5, Cy5, Cy5.5, Cy7, Dabcyl, Edans, Eosin, Erythrosin, Fuorescein*, 6-FAM™, TET™, JOE, HEX, LightCycler 640, LightCycler 705, NBD, Oregon Green 488, Oregon Green 500, Oregon Green 514, Rhodamine 6G, Rhodamine Green, Rhodamine Red, Rhodol Green, TAMRA™, ROX, Texas Red, VIC® and NED™. Compatible Quenchers include but are not limited to TAMRA (6-carboxytetramethylrhodamine) and a non-fluorescent quencher dye attached to an minor groove binding moiety (MGB).

A “kit” as used herein refers to a set of reagents for the purpose of performing the method embodiments of the disclosure, more particularly, the identification of event DP-032218-9 in biological samples. The kit of the disclosure can be used, and its components can be specifically adjusted, for purposes of quality control (e.g. purity of seed lots), detection of event DP-032218-9 in plant material, or material comprising or derived from plant material, such as but not limited to food or feed products. “Plant material” as used herein refers to material which is obtained or derived from a plant.

Primers and probes based on the flanking DNA and insert sequences disclosed herein can be used to confirm (and, if necessary, to correct) the disclosed sequences by conventional methods, e.g., by re-cloning and sequencing such sequences. The nucleic acid probes and primers of the present disclosure hybridize under stringent conditions to a target DNA sequence. Any conventional nucleic acid hybridization or amplification method can be used to identify the presence of DNA from a transgenic event in a sample. Nucleic acid molecules or fragments thereof are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure.

A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., 1989, and by Haymes et al., In: Nucleic Acid Hybridization, a Practical Approach, IRL Press, Washington, D.C. (1985). Departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed.

In hybridization reactions, specificity is typically the function of post-hybridization washes, the critical factors being the ionic strength and temperature of the final wash solution. The thermal melting point (Tₘ) is the temperature (under defined ionic strength and pH) at which 50% of a complementary target sequence hybridizes to a perfectly matched probe. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ.

Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) and Sambrook et al. (1989).

As used herein, a substantially homologous sequence is a nucleic acid molecule that will specifically hybridize to the complement of the nucleic acid molecule to which it is being compared under high stringency conditions. Appropriate stringency conditions which promote DNA hybridization, for example, 6× sodium chloride/sodium citrate (SSC) at about 45° C., followed by a wash of 2×SSC at 50° C., are known to those skilled in the art or can be found in Ausubel et al. (1995), 6.3.1-6.3.6. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of a destabilizing agent such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. A nucleic acid of the disclosure may specifically hybridize to one or more of the nucleic acid molecules unique to the DP-032218-9 event or complements thereof or fragments of either under moderately stringent conditions.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller (1988) CABIOS4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 87:2264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0); the ALIGN PLUS program (version 3.0, copyright 1997); and GAP, BESTFIT, BLAST, FASTA, and TFASTA in the Wisconsin Genetics Software Package, Version 10 (available from Accelrys, 9685 Scranton Road, San Diego, Calif. 92121, USA). Alignments using these programs can be performed using the default parameters.

The CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994). The ALIGN and the ALIGN PLUS programs are based on the algorithm of Myers and Miller (1988) supra. The BLAST programs of Altschul et al. (1990) J. Mol. Biol. 215:403 are based on the algorithm of Karlin and Altschul (1990) supra. The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Ausubel, et al., (1995). Alignment may also be performed manually by visual inspection.

To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used.

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Regarding the amplification of a target nucleic acid sequence (e.g., by PCR) using a particular amplification primer pair, “stringent conditions” are conditions that permit the primer pair to hybridize only to the target nucleic-acid sequence to which a primer having the corresponding wild-type sequence (or its complement) would bind and preferably to produce a unique amplification product, the amplicon, in a DNA thermal amplification reaction.

The term “specific for (a target sequence)” indicates that a probe or primer hybridizes under stringent hybridization conditions only to the target sequence in a sample comprising the target sequence.

As used herein, “amplified DNA” or “amplicon” refers to the product of nucleic acid amplification of a target nucleic acid sequence that is part of a nucleic acid template. For example, to determine whether a corn plant resulting from a sexual cross contains transgenic event genomic DNA from the corn plant of the disclosure, DNA extracted from the corn plant tissue sample may be subjected to a nucleic acid amplification method using a DNA primer pair that includes a first primer derived from flanking sequence adjacent to the insertion site of inserted heterologous DNA, and a second primer derived from the inserted heterologous DNA to produce an amplicon that is diagnostic for the presence of the event DNA. Alternatively, the second primer may be derived from the flanking sequence. The amplicon is of a length and has a sequence that is also diagnostic for the event. The amplicon may range in length from the combined length of the primer pairs plus one nucleotide base pair to any length of amplicon producible by a DNA amplification protocol. Alternatively, primer pairs can be derived from flanking sequence on both sides of the inserted DNA so as to produce an amplicon that includes the entire insert nucleotide sequence of the PHP36676 expression construct as well as the sequence flanking the transgenic insert. A member of a primer pair derived from the flanking sequence may be located a distance from the inserted DNA sequence, this distance can range from one nucleotide base pair up to the limits of the amplification reaction, or about 20,000 bp. The use of the term “amplicon” specifically excludes primer dimers that may be formed in the DNA thermal amplification reaction.

Nucleic acid amplification can be accomplished by any of the various nucleic acid amplification methods known in the art, including PCR. A variety of amplification methods are known in the art and are described, inter alia, in U.S. Pat. Nos. 4,683,195 and 4,683,202 and in Innis et al., (1990) supra. PCR amplification methods have been developed to amplify up to 22 Kb of genomic DNA and up to 42 Kb of bacteriophage DNA (Cheng et al., Proc. Natl. Acad. Sci. USA 91:5695-5699, 1994). These methods as well as other methods known in the art of DNA amplification may be used in the practice of the embodiments of the present disclosure. It is understood that a number of parameters in a specific PCR protocol may need to be adjusted to specific laboratory conditions and may be slightly modified and yet allow for the collection of similar results. These adjustments will be apparent to a person skilled in the art.

The amplicon produced by these methods may be detected by a plurality of techniques, including, but not limited to, Genetic Bit Analysis (Nikiforov, et al. Nucleic Acid Res. 22:4167-4175, 1994) where a DNA oligonucleotide is designed which overlaps both the adjacent flanking DNA sequence and the inserted DNA sequence. The oligonucleotide is immobilized in wells of a microwell plate. Following PCR of the region of interest (using one primer in the inserted sequence and one in the adjacent flanking sequence) a single-stranded PCR product can be hybridized to the immobilized oligonucleotide and serve as a template for a single base extension reaction using a DNA polymerase and labeled ddNTPs specific for the expected next base. Readout may be fluorescent or ELISA-based. A signal indicates presence of the insert/flanking sequence due to successful amplification, hybridization, and single base extension.

Another detection method is the pyrosequencing technique as described by Winge (2000) Innov. Pharma. Tech. 00:18-24. In this method an oligonucleotide is designed that overlaps the adjacent DNA and insert DNA junction. The oligonucleotide is hybridized to a single-stranded PCR product from the region of interest (one primer in the inserted sequence and one in the flanking sequence) and incubated in the presence of a DNA polymerase, ATP, sulfurylase, luciferase, apyrase, adenosine 5′ phosphosulfate and luciferin. dNTPs are added individually and the incorporation results in a light signal which is measured. A light signal indicates the presence of the transgene insert/flanking sequence due to successful amplification, hybridization, and single or multi-base extension.

Fluorescence polarization as described by Chen et al., (1999) Genome Res. 9:492-498 is also a method that can be used to detect an amplicon of the disclosure. Using this method an oligonucleotide is designed which overlaps the flanking and inserted DNA junction. The oligonucleotide is hybridized to a single-stranded PCR product from the region of interest (one primer in the inserted DNA and one in the flanking DNA sequence) and incubated in the presence of a DNA polymerase and a fluorescent-labeled ddNTP. Single base extension results in incorporation of the ddNTP. Incorporation can be measured as a change in polarization using a fluorometer. A change in polarization indicates the presence of the transgene insert/flanking sequence due to successful amplification, hybridization, and single base extension.

Taqman® (PE Applied Biosystems, Foster City, Calif.) is described as a method of detecting and quantifying the presence of a DNA sequence and is fully understood in the instructions provided by the manufacturer. Briefly, a FRET oligonucleotide probe is designed which overlaps the flanking and insert DNA junction. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization.

Molecular beacons have been described for use in sequence detection as described in Tyangi et al. (1996) Nature Biotech. 14:303-308. Briefly, a FRET oligonucleotide probe is designed that overlaps the flanking and insert DNA junction. The unique structure of the FRET probe results in it containing secondary structure that keeps the fluorescent and quenching moieties in close proximity. The FRET probe and PCR primers (one primer in the insert DNA sequence and one in the flanking sequence) are cycled in the presence of a thermostable polymerase and dNTPs. Following successful PCR amplification, hybridization of the FRET probe to the target sequence results in the removal of the probe secondary structure and spatial separation of the fluorescent and quenching moieties. A fluorescent signal results. A fluorescent signal indicates the presence of the flanking/transgene insert sequence due to successful amplification and hybridization.

A hybridization reaction using a probe specific to a sequence found within the amplicon is yet another method used to detect the amplicon produced by a PCR reaction.

Maize event DP-032218-9 is effective against insect pests including insects selected from the orders: Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera.

Insects of the order Lepidoptera include, but are not limited to, armyworms, cutworms, loopers, and heliothines in the family Noctuidae: Agrotis ipsilon Hufnagel (black cutworm); A. orthogonia Morrison (western cutworm); A. segetum Denis & Schiffermüller (turnip moth); A. subterranea Fabricius (granulate cutworm); Alabama argillacea Hübner (cotton leaf worm); Anticarsia gemmatalis Hübner (velvetbean caterpillar); Athetis mindara Barnes and McDunnough (rough skinned cutworm); Earias insulana Boisduval (spiny bollworm); E. vittella Fabricius (spotted bollworm); Egira (Xylomyges) curialis Grote (citrus cutworm); Euxoa messoria Harris (darksided cutworm); Helicoverpa armigera Hübner (American bollworm); H. zea Boddie (corn earworm or cotton bollworm); Heliothis virescens Fabricius (tobacco budworm); Hypena scabra Fabricius (green cloverworm); Hyponeuma taltula Schaus; (Mamestra configurata Walker (bertha armyworm); M. brassicae Linnaeus (cabbage moth); Melanchra picta Harris (zebra caterpillar); Mocis latipes Guenée (small mocis moth); Pseudaletia unipuncta Haworth (armyworm); Pseudoplusia includens Walker (soybean looper); Richia albicosta Smith (Western bean cutworm); Spodoptera frugiperda JE Smith (fall armyworm); S. exigua Hübner (beet armyworm); S. litura Fabricius (tobacco cutworm, cluster caterpillar); Trichoplusia ni Hübner (cabbage looper); borers, casebearers, webworms, coneworms, and skeletonizers from the families Pyralidae and Crambidae such as Achroia grisella Fabricius (lesser wax moth); Amyelois transitella Walker (naval orangeworm); Anagasta kuehniella Zeller (Mediterranean flour moth); Cadra cautella Walker (almond moth); Chilo partellus Swinhoe (spotted stalk borer); C. suppressalis Walker (striped stem/rice borer); C. terrenellus Pagenstecher (sugarcane stem borer); Corcyra cephalonica Stainton (rice moth); Crambus caliginosellus Clemens (corn root webworm); C. teterrellus Zincken (bluegrass webworm); Cnaphalocrocis medinalis Guenée (rice leaf roller); Desmia funeralis Hübner (grape leaffolder); Diaphania hyalinata Linnaeus (melon worm); D. nitidalis Stoll (pickleworm); Diatraea flavipennella Box; D. grandiosella Dyar (southwestern corn borer), D. saccharalis Fabricius (surgarcane borer); Elasmopalpus lignosellus Zeller (lesser cornstalk borer); Eoreuma loftini Dyar (Mexican rice borer); Ephestia elutella Hübner (tobacco (cacao) moth); Galleria mellonella Linnaeus (greater wax moth); Hedylepta accepta Butler (sugarcane leafroller); Herpetogramma licarsisalis Walker (sod webworm); Homoeosoma electellum Hulst (sunflower moth); Loxostege sticticalis Linnaeus (beet webworm); Maruca testulalis Geyer (bean pod borer); Orthaga thyrisalis Walker (tea tree web moth); Ostrinia nubilalis Hübner (European corn borer); Plodia interpunctella Hübner (Indian meal moth); Scirpophaga incertulas Walker (yellow stem borer); Udea rubigalis Guenée (celery leaftier); and leafrollers, budworms, seed worms, and fruit worms in the family Tortricidae Acleris gloverana Walsingham (Western blackheaded budworm); A. variana Fernald (Eastern blackheaded budworm); Adoxophyes orana Fischer von Rösslerstamm (summer fruit tortrix moth); Archips spp. including A. argyrospila Walker (fruit tree leaf roller) and A. rosana Linnaeus (European leaf roller); Argyrotaenia spp.; Bonagota salubricola Meyrick (Brazilian apple leafroller); Choristoneura spp.; Cochylis hospes Walsingham (banded sunflower moth); Cydia latiferreana Walsingham (filbertworm); C. pomonella Linnaeus (codling moth); Endopiza viteana Clemens (grape berry moth); Eupoecilia ambiguella Hübner (vine moth); Grapholita molesta Busck (oriental fruit moth); Lobesia botrana Denis & Schiffermüller (European grape vine moth); Platynota flavedana Clemens (variegated leafroller); P. stultana Walsingham (omnivorous leafroller); Spilonota ocellana Denis & Schiffermüller (eyespotted bud moth); and Suleima helianthana Riley (sunflower bud moth).

Selected other agronomic pests in the order Lepidoptera include, but are not limited to, Alsophila pometaria Harris (fall cankerworm); Anarsia lineatella Zeller (peach twig borer); Anisota senatoria J. E. Smith (orange striped oakworm); Antheraea pernyi Guérin-Méneville (Chinese Oak Silkmoth); Bombyx mori Linnaeus (Silkworm); Bucculatrix thurberiella Busck (cotton leaf perforator); Colias eurytheme Boisduval (alfalfa caterpillar); Datana integerrima Grote & Robinson (walnut caterpillar); Dendrolimus sibiricus Tschetwerikov (Siberian silk moth), Ennomos subsignaria Hübner (elm spanworm); Erannis tiliaria Harris (linden looper); Erechthias flavistriata Walsingham (sugarcane bud moth); Euproctis chrysorrhoea Linnaeus (browntail moth); Harrisina americana Guérin-Méneville (grapeleaf skeletonizer); Heliothis subflexa Guenée; Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria lugubrosa Hulst (Western hemlock looper); Leucoma salicis Linnaeus (satin moth); Lymantria dispar Linnaeus (gypsy moth); Malacosoma spp.; Manduca quinquemaculata Haworth (five spotted hawk moth, tomato hornworm); M. sexta Haworth (tomato hornworm, tobacco hornworm); Operophtera brumata Linnaeus (winter moth); Orgyia spp.; Paleacrita vernata Peck (spring cankerworm); Papilio cresphontes Cramer (giant swallowtail, orange dog); Phryganidia californica Packard (California oakworm); Phyllocnistis citrella Stainton (citrus leafminer); Phyllonorycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval & Leconte (Southern cabbageworm); Sabulodes aegrotata Guenée (omnivorous looper); Schizura concinna J. E. Smith (red humped caterpillar); Sitotroga cerealella Olivier (Angoumois grain moth); Telchin licus Drury (giant sugarcane borer); Thaumetopoea pityocampa Schiffermiller (pine processionary caterpillar); Tineola bisselliella Hummel (webbing clothesmoth); Tuta absoluta Meyrick (tomato leafminer) and Yponomeuta padella Linnaeus (ermine moth).

Of interest are larvae and adults of the order Coleoptera including weevils from the families Anthribidae, Bruchidae, and Curculionidae including, but not limited to: Anthonomus grandis Boheman (boll weevil); Cylindrocopturus adspersus LeConte (sunflower stem weevil); Diaprepes abbreviatus Linnaeus (Diaprepes root weevil); Hypera punctata Fabricius (clover leaf weevil); Lissorhoptrus oryzophilus Kuschel (rice water weevil); Metamasius hemipterus hemipterus Linnaeus (West Indian cane weevil); M. hemipterus sericeus Olivier (silky cane weevil); Sitophilus granarius Linnaeus (granary weevil); S. oryzae Linnaeus (rice weevil); Smicronyx fulvus LeConte (red sunflower seed weevil); S. sordidus LeConte (gray sunflower seed weevil); Sphenophorus maidis Chittenden (maize billbug); S. livis Vaurie (sugarcane weevil); Rhabdoscelus obscurus Boisduval (New Guinea sugarcane weevil); flea beetles, cucumber beetles, rootworms, leaf beetles, potato beetles, and leafminers in the family Chrysomelidae including, but not limited to: Chaetocnema ectypa Horn (desert corn flea beetle); C. pulicaria Melsheimer (corn flea beetle); Colaspis brunnea Fabricius (grape colaspis); Diabrotica barberi Smith & Lawrence (northern corn rootworm); D. undecimpunctata howardi Barber (southern corn rootworm); D. virgifera virgifera LeConte (western corn rootworm); Leptinotarsa decemlineata Say (Colorado potato beetle); Oulema melanopus Linnaeus (cereal leaf beetle); Phyllotreta cruciferae Goeze (corn flea beetle); Zygogramma exclamationis Fabricius (sunflower beetle); beetles from the family Coccinellidae including, but not limited to: Epilachna varivestis Mulsant (Mexican bean beetle); chafers and other beetles from the family Scarabaeidae including, but not limited to: Antitrogus parvulus Britton (Childers cane grub); Cyclocephala borealis Arrow (northern masked chafer, white grub); C. immaculata Olivier (southern masked chafer, white grub); Dermolepida albohirtum Waterhouse (Greyback cane beetle); Euetheola humilis rugiceps LeConte (sugarcane beetle); Lepidiota frenchi Blackburn (French's cane grub); Tomarus gibbosus De Geer (carrot beetle); T. subtropicus Blatchley (sugarcane grub); Phyllophaga crinita Burmeister (white grub); P. latifrons LeConte (June beetle); Popillia japonica Newman (Japanese beetle); Rhizotrogus majalis Razoumowsky (European chafer); carpet beetles from the family Dermestidae; wireworms from the family Elateridae, Eleodes spp., Melanotus spp. including M. communis Gyllenhal (wireworm); Conoderus spp.; Limonius spp.; Agriotes spp.; Ctenicera spp.; Aeolus spp.; bark beetles from the family Scolytidae; beetles from the family Tenebrionidae; beetles from the family Cerambycidae such as, but not limited to, Migdolus fryanus Westwood (longhorn beetle); and beetles from the Buprestidae family including, but not limited to, Aphanisticus cochinchinae seminulum Obenberger (leaf-mining buprestid beetle).

Adults and immatures of the order Diptera are of interest, including leafminers Agromyza parvicornis Loew (corn blotch leafminer); midges including, but not limited to: Contarinia sorghicola Coquillett (sorghum midge); Mayetiola destructor Say (Hessian fly); Neolasioptera murtfeldtiana Felt, (sunflower seed midge); Sitodiplosis mosellana Géhin (wheat midge); fruit flies (Tephritidae), Oscinella frit Linnaeus (frit flies); maggots including, but not limited to: Delia spp. including Delia platura Meigen (seedcorn maggot); D. coarctata Fallen (wheat bulb fly); Fannia canicularis Linnaeus, F. femoralis Stein (lesser house flies); Meromyza americana Fitch (wheat stem maggot); Musca domestica Linnaeus (house flies); Stomoxys calcitrans Linnaeus (stable flies)); face flies, horn flies, blow flies, Chrysomya spp.; Phormia spp.; and other muscoid fly pests, horse flies Tabanus spp.; bot flies Gastrophilus spp.; Oestrus spp.; cattle grubs Hypoderma spp.; deer flies Chrysops spp.; Melophagus ovinus Linnaeus (keds); and other Brachycera, mosquitoes Aedes spp.; Anopheles spp.; Culex spp.; black flies Prosimulium spp.; Simulium spp.; biting midges, sand flies, sciarids, and other Nematocera.

Included as insects of interest are those of the order Hemiptera such as, but not limited to, the following families: Adelgidae, Aleyrodidae, Aphididae, Asterolecaniidae, Cercopidae, Cicadellidae, Cicadidae, Cixiidae, Coccidae, Coreidae, Dactylopiidae, Delphacidae, Diaspididae, Eriococcidae, Flatidae, Fulgoridae, Issidae, Lygaeidae, Margarodidae, Membracidae, Miridae, Ortheziidae, Pentatomidae, Phoenicococcidae, Phylloxeridae, Pseudococcidae, Psyllidae, Pyrrhocoridae and Tingidae.

Agronomically important members from the order Hemiptera include, but are not limited to: Acrosternum hilare Say (green stink bug); Acyrthisiphon pisum Harris (pea aphid); Adelges spp. (adelgids); Adelphocoris rapidus Say (rapid plant bug); Anasa tristis De Geer (squash bug); Aphis craccivora Koch (cowpea aphid); A. fabae Scopoli (black bean aphid); A. gossypii Glover (cotton aphid, melon aphid); A. maidiradicis Forbes (corn root aphid); A. pomi De Geer (apple aphid); A. spiraecola Patch (spirea aphid); Aulacaspis tegalensis Zehntner (sugarcane scale); Aulacorthum solani Kaltenbach (foxglove aphid); Bemisia tabaci Gennadius (tobacco whitefly, sweetpotato whitefly); B. argentifolii Bellows & Perring (silverleaf whitefly); Blissus leucopterus leucopterus Say (chinch bug); Blostomatidae spp.; Brevicoryne brassicae Linnaeus (cabbage aphid); Cacopsylla pyricola Foerster (pear psylla); Calocoris norvegicus Gmelin (potato capsid bug); Chaetosiphon fragaefolii Cockerell (strawberry aphid); Cimicidae spp.; Coreidae spp.; Corythuca gossypii Fabricius (cotton lace bug); Cyrtopeltis modesta Distant (tomato bug); C. notatus Distant (suckfly); Deois flavopicta Stal (spittlebug); Dialeurodes citri Ashmead (citrus whitefly); Diaphnocoris chlorionis Say (honeylocust plant bug); Diuraphis noxia Kurdjumov/Mordvilko (Russian wheat aphid); Duplachionaspis divergens Green (armored scale); Dysaphis plantaginea Paaserini (rosy apple aphid); Dysdercus suturellus Herrich-Schfiffer (cotton stainer); Dysmicoccus boninsis Kuwana (gray sugarcane mealybug); Empoasca fabae Harris (potato leafhopper); Eriosoma lanigerum Hausmann (woolly apple aphid); Erythroneoura spp. (grape leafhoppers); Eumetopina flavipes Muir (Island sugarcane planthopper); Eurygaster spp.; Euschistus servus Say (brown stink bug); E. variolarius Palisot de Beauvois (one-spotted stink bug); Graptostethus spp. (complex of seed bugs); and Hyalopterus pruni Geoffroy (mealy plum aphid); Icerya purchasi Maskell (cottony cushion scale); Labopidicola allii Knight (onion plant bug); Laodelphax striatellus Fallen (smaller brown planthopper); Leptoglossus corculus Say (leaf-footed pine seed bug); Leptodictya tabida Herrich-Schaeffer (sugarcane lace bug); Lipaphis erysimi Kaltenbach (turnip aphid); Lygocoris pabulinus Linnaeus (common green capsid); Lygus lineolaris Palisot de Beauvois (tarnished plant bug); L. Hesperus Knight (Western tarnished plant bug); L. pratensis Linnaeus (common meadow bug); L. rugulipennis Poppius (European tarnished plant bug); Macrosiphum euphorbiae Thomas (potato aphid); Macrosteles quadrilineatus Forbes (aster leafhopper); Magicicada septendecim Linnaeus (periodical cicada); Mahanarva fimbriolata Stal (sugarcane spittlebug); M. posticata Stal (little cicada of sugarcane); Melanaphis sacchari Zehntner (sugarcane aphid); Melanaspis glomerata Green (black scale); Metopolophium dirhodum Walker (rose grain aphid); Myzus persicae Sulzer (peach-potato aphid, green peach aphid); Nasonovia ribisnigri Mosley (lettuce aphid); Nephotettix cinticeps Uhler (green leafhopper); N. nigropictus Stal (rice leafhopper); Nezara viridula Linnaeus (southern green stink bug); Nilaparvata lugens Stal (brown planthopper); Nysius ericae Schilling (false chinch bug); Nysius raphanus Howard (false chinch bug); Oebalus pugnax Fabricius (rice stink bug); Oncopeltus fasciatus Dallas (large milkweed bug); Orthops campestris Linnaeus; Pemphigus spp. (root aphids and gall aphids); Peregrinus maidis Ashmead (corn planthopper); Perkinsiella saccharicida Kirkaldy (sugarcane delphacid); Phylloxera devastatrix Pergande (pecan phylloxera); Planococcus citri Risso (citrus mealybug); Plesiocoris rugicollis Fallen (apple capsid); Poecilocapsus lineatus Fabricius (four-lined plant bug); Pseudatomoscelis seriatus Reuter (cotton fleahopper); Pseudococcus spp. (other mealybug complex); Pulvinaria elongata Newstead (cottony grass scale); Pyrilla perpusilla Walker (sugarcane leafhopper); Pyrrhocoridae spp.; Quadraspidiotus perniciosus Comstock (San Jose scale); Reduviidae spp.; Rhopalosiphum maidis Fitch (corn leaf aphid); R. padi Linnaeus (bird cherry-oat aphid); Saccharicoccus sacchari Cockerell (pink sugarcane mealybug); Scaptocoris castanea Perty (brown root stink bug); Schizaphis graminum Rondani (greenbug); Sipha flava Forbes (yellow sugarcane aphid); Sitobion avenae Fabricius (English grain aphid); Sogatella furcifera Horvath (white-backed planthopper); Sogatodes oryzicola Muir (rice delphacid); Spanagonicus albofasciatus Reuter (whitemarked fleahopper); Therioaphis maculata Buckton (spotted alfalfa aphid); Tinidae spp.; Toxoptera aurantii Boyer de Fonscolombe (black citrus aphid); and T. citricida Kirkaldy (brown citrus aphid); Trialeurodes abutiloneus (bandedwinged whitefly) and T. vaporariorum Westwood (greenhouse whitefly); Trioza diospyri Ashmead (persimmon psylla); and Typhlocyba pomaria McAtee (white apple leafhopper).

Also included are adults and larvae of the order Acari (mites) such as Aceria tosichella Keifer (wheat curl mite); Panonychus ulmi Koch (European red mite); Petrobia latens Müller (brown wheat mite); Steneotarsonemus bancrofti Michael (sugarcane stalk mite); spider mites and red mites in the family Tetranychidae, Oligonychus grypus Baker & Pritchard, O. indicus Hirst (sugarcane leaf mite), O. pratensis Banks (Banks grass mite), O. stickneyi McGregor (sugarcane spider mite); Tetranychus urticae Koch (two spotted spider mite); T. mcdanieli McGregor (McDaniel mite); T. cinnabarinus Boisduval (carmine spider mite); T. turkestani Ugarov & Nikolski (strawberry spider mite), flat mites in the family Tenuipalpidae, Brevipalpus lewisi McGregor (citrus flat mite); rust and bud mites in the family Eriophyidae and other foliar feeding mites and mites important in human and animal health, i.e. dust mites in the family Epidermoptidae, follicle mites in the family Demodicidae, grain mites in the family Glycyphagidae, ticks in the order Ixodidae. Ixodes scapularis Say (deer tick);/. holocyclus Neumann (Australian paralysis tick); Dermacentor variabilis Say (American dog tick); Amblyomma americanum Linnaeus (lone star tick); and scab and itch mites in the families Psoroptidae, Pyemotidae, and Sarcoptidae.

Insect pests of the order Thysanura are of interest, such as Lepisma saccharina Linnaeus (silverfish); Thermobia domestica Packard (firebrat).

Additional arthropod pests covered include: spiders in the order Araneae such as Loxosceles reclusa Gertsch & Mulaik (brown recluse spider); and the Latrodectus mactans Fabricius (black widow spider); and centipedes in the order Scutigeromorpha such as Scutigera coleoptrata Linnaeus (house centipede). In addition, insect pests of the order Isoptera are of interest, including those of the Termitidae family, such as, but not limited to, Cornitermes cumulans Kollar, Cylindrotermes nordenskioeldi Holmgren and Pseudacanthotermes militaris Hagen (sugarcane termite); as well as those in the Rhinotermitidae family including, but not limited to Heterotermes tenuis Hagen. Insects of the order Thysanoptera are also of interest, including but not limited to thrips, such as Stenchaetothrips minutus van Deventer (sugarcane thrips).

Embodiments of the present disclosure are further defined in the following Examples. It should be understood that these Examples are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

The disclosure of each reference set forth herein is incorporated by reference in its entirety.

### EXAMPLES

### Example 1. Transformation of Maize by Agrobacterium Transformation and Regeneration of Transgenic Plants Containing the vip3Aa20, cry2A.127, cry1A.88, and Mo-Pat Genes

Maize (Zea mays L.) event DP-032218-9 was produced by Agrobacterium-mediated transformation with plasmid PHP36676. The T-DNA region of the plasmid sequence is provided in SEQ ID NO: 1. A summary of the genetic elements and their positions on plasmid PHP36676 and on the T-DNA are described in Table 2.

The T-DNA of plasmid PHP36676 contains four gene cassettes. The first cassette contains the proprietary cry2A. 127 gene, a Cry2Ab-like coding sequence that has been functionally optimized using DNA shuffling and directed mutagenesis techniques. The 634 residue protein produced by expression of the cry2A. 127 sequence is targeted to maize chloroplasts through the addition of a 56 amino acid codon-optimized synthetic chloroplast targeting peptide (CTP) as well as 4 synthetic linker amino acids, resulting in a total length of 694 amino acids (approximately 77 kDa) for the precursor protein (the Cry2A.127 CTP sequence is cleaved upon insertion into the chloroplast, resulting in a mature protein of approximately 71 kDa. The expression of the cry2A. 127 gene and attached transit peptide is controlled by the Citus Yellow Mosaic Virus (CYMV; Genbank accession AF347695.1) promoter along with a downstream copy of the maize adh1 intron (Dennis et al., 1984). Transcription of the cry2A. 127 gene cassette is terminated by the downstream presence of the Arabidopsis thaliana ubiquitin 3 (UBQ3) termination region (Callis et al., 1995). In addition, a 2.2 kB fragment corresponding to the 3′ un-translated region from an Arabidopsis ribosomal protein gene (TAIR accession AT3G28500; Salanoubat et al., 2000) is located between the cry2A.127 and cry1A.88 cassettes in order to eliminate any potential read thru transcripts.

The second cassette contains a second shuffled proprietary insect control gene, the Cry1A-like cry1A.88 coding region. This 1182 residue coding region (which produces a precursor protein of approximately 133 kDa, is controlled by a truncated version (470 nucleotides in length) of the full length promoter from Banana Streak Virus (Acuminata Vietnam strain; Lheureux et al., 2007) along with a second copy of the maize adh1 intron. The termination region for the cry1A.88 cassette is a 1.1 kB portion of the Sorghum bi-color genome containing the 3′ termination region from the SB-Actin gene (Paterson et al., 2009)). Three other termination regions are present between the second and third cassettes; the 27 kD gamma zein terminator originally isolated from maize line W64A (Das et al., 1991), a genomic fragment of Arabidopsis thaliana chromosome 4 containing the Ubiquitin-14 (UBQ14) 3′UTR and terminator (Mayer et al., 1999) and the termination sequence from the maize In2-1 gene (Hershey and Stoner, 1991).

The third cassette contains the vip3Aa20 gene, which codes for a synthetic version of the insecticidal Vip3Aa20 protein (present in the approved Syngenta event MIR162; Estruch et al., 1996). Expression of the vip3Aa20 gene is controlled by the the maize polyubiquitin promoter, including the 5′ untranslated region and intron 1 (Christensen et al., 1992). The terminator for the vip3Aa20 gene is the 3′ terminator sequence from the proteinase inhibitor II gene of Solanum tuberosum (pinII terminator) (Keil et al., 1986; An et al., 1989). The Vip3Aa20 protein is 789 amino acid residues in length with an approximate molecular weight of 88 kDa.

The fourth and final gene cassette contains a version of the phosphinothricin acetyl transferase gene (mo-pat) from Streptomyces viridochromogenes (Wohlleben et al., 1988) that has been optimized for expression in maize. The pat gene expresses the phosphinothricin acetyl transferase enzyme (PAT) that confers tolerance to phosphinothricin. The PAT protein is 183 amino acids residues in length and has a molecular weight of approximately 21 kDa. Expression of the mo-pat gene is controlled by a second copy of the maize polyubiquitin promoter/5′UTR/intron in conjunction with a second copy of the pinII terminator.

TABLE 2
Genetic Elements in the T-DNA Region of Plasmid PHP36676

| Location on | | Size | |
|---|---|---|---|
| T-DNA (base | | (base | |
| pair position) | Genetic Element | pairs) | Description |
| 1-25 | Right Border | 25 | T-DNA Right Border region from Ti plasmid |
| | | | of Agrobacterium tumefaciens |
| 26-305 | Ti Plasmid Region | 279 | Non-functional sequence from Ti plasmid of A. tumefaciens |
| 306-317 | Mini all stops | 12 | Artificial sequence containing stop codons in all |
| | | | 6 reading frames |
| 318-429 | PSA2 | 112 | A synthetic sequence designed to facilitate PCR |
| | | | analysis of recombined FRT sites |
| 436-469 | loxP site | 34 | bacteriophage P1 recombination site recognized |
| | | | by Cre recombinase (Dale and Ow, 1990) |
| 697-758 | attB3 site | 62 | Bacteriophage lambda integrase recombination |
| | | | site (Cheo et al., 2004) |
| 759-1911 | CYMV promoter | 1153 | Promoter from Citrus Yellow Mosaic Virus |
| | | | (CYMV) (Huang and Hartung, 2001; Genbank |
| | | | accession NC_003382.1) |
| 1939-2481 | adh1 Intron | 543 | Intron 1 from the maize alcohol dehydrogenase |
| | | | gene (Dennis et al., 1984) |
| 2496-2657 | Chloroplast Transit | 162 | Fifty six residue synthetic peptide that allows |
| | Peptide (CTP) | | targeting of mature cry2A.127 gene product to |
| | (complementary) | | the chloroplast (cleaved from the mature |
| | | | protein) |
| 2676-4580 | cry2A.127 gene | 1905 | Cry2A-like coding sequence that has been |
| | (complementary) | | functionally optimized using DNA shuffling |
| | | | and directed mutagenesis techniques |
| 4611-5699 | UBQ3 Terminator | 1089 | Transcription termination region from the |
| | | | ubiquitin 3 gene of Arabidopsis thaliana (Callis et |
| | | | al., 1995) |
| 5705-7932 | RPG 3′ UTR | 2227 | 3′untranslated region from an Arabidopsis |
| | | | ribosomal protein gene (AT3G28500; |
| | | | Salanoubat et al., 2000) |
| 8096-8119 | attB2 | 24 | Bacteriophage lambda integrase recombination |
| | | | site (Hartley et al., 2000) |
| 8139-8172 | All Stops | 34 | Artificial sequence containing stop codons in all |
| | | | 6 reading frames |
| 8183-8652 | BSV (AV) | 470 | A truncated version of the genomic promoter |
| | Promoter | | from Banana Streak Virus (Acuminata Vietnam |
| | | | strain; Lheureux et al., 2007) |
| 8680-9222 | adh1 Intron | 543 | Intron 1 from the maize alcohol dehydrogenase |
| | | | gene (Dennis et al., 1984) |
| 9237-12785 | cry1A.88 gene | 3,549 | A Cry1A-type coding sequence (including |
| | (complementary) | | protoxin regions) that has been functionally |
| | | | optimized using DNA shuffling and directed |
| | | | mutagenesis techniques |
| 12804-13846 | SB-Actin | 1,043 | Portion of sorghum chr9 containing the 3′ |
| | Terminator | | termination region from SB-Actin gene |
| | | | (Paterson et al., 2009) |
| 13880-14359 | GZ-W64A | 480 | Maize 27 kD gamma zein terminator, isolated |
| | Terminator | | from W64A line (Das et al., 1991) |
| 14366-15267 | UBQ14 Terminator | 902 | Fragment of Arabidopsis thaliana. chromosome 4 |
| | | | containing the Ubiquitin-14 (UBQ14) 3′UTR |
| | | | and terminator (Mayer et al., 1999) |
| 15274-15767 | ln2-1 Terminator | 494 | Terminator sequence from the maize In2-1 gene |
| | | | (Hershey and Stoner, 1991). |
| 15818-15851 | All Stops | 6 | Artificial sequence containing stop codons in all |
| | | | 6 reading frames |
| 15857-15880 | attB1 site | 24 | Bacteriophage lambda integrase recombination |
| | | | site (Hartley et al., 2000) |
| 15964-16863 | ubiZM1 Promoter | 900 | Promoter region from Zea mays polyubiquitin |
| | | | gene (Christensen et al., 1992) |
| 16864-16946 | ubiZM1 5′UTR | 83 | 5′ untranslated region from Zea mays |
| | | | polyubiquitin gene (Christensen et al., 1992) |
| 16947-17959 | ubiZM1 Intron | 1,013 | Intron region from Zea mays polyubiquitin gene |
| | | | (Christensen et al., 1992) |
| 17986-20355 | vip3Aa20 gene | 2370 | Synthetic version of insecticidal VIP3A protein |
| | (complementary) | | (Estruch et al., 1996) |
| 20362-20671 | pinII Terminator | 310 | Terminator region from Solanum tuberosum |
| | | | proteinase inhibitor II gene (Keil et al., 1986; An |
| | | | et al., 1989). |
| 20792-20812 | attB4 site | 21 | Bacteriophage lambda integrase recombination |
| | | | site (Hartley et al., 2000) |
| 20888-20921 | loxP site | 34 | bacteriophage P1 recombination site recognized |
| | | | by Cre recombinase (Dale and Ow, 1990) |
| 20941-21840 | ubiZM1 Promoter | 900 | Promoter region from Zea mays polyubiquitin |
| | | | gene (Christensen et al., 1992) |
| 21841-21923 | ubiZM1 5′UTR | 83 | 5′ untranslated region from Zea mays |
| | | | polyubiquitin gene (Christensen et al., 1992) |
| 21924-22936 | ubiZM1 Intron | 1,013 | Intron region from Zea mays polyubiquitin gene |
| | | | (Christensen et al., 1992) |
| 22965-23012 | FRT1 site | 48 | Flp recombinase DNA binding site (Pan et al., |
| | | | 1991) |
| 23039-23590 | mo-pat gene | 552 | Maize optimized version of the |
| | | | phosphinothricin acetyl transferase gene (pat) |
| | | | from Streptomyces viridochromogenes (Wohlleben et |
| | | | al., 1988) |
| 23599-23908 | pinII Terminator | 310 | Terminator region from Solanum tuberosum |
| | | | proteinase inhibitor II gene (Keil et al., 1986; An |
| | | | et al., 1989). |
| 23930-23977 | FRT87 site | 48 | Modified Flp recombinase DNA binding site |
| | | | (Tao et al., 2007) |
| 24001-24095 | PSB1 site | 95 | Synthetic sequence designed to facilitate PCR |
| | | | analysis of recombined FRT sites. |
| 24096-24107 | Mini all stops | 12 | Artificial sequence containing stop codons in all |
| | | | 6 reading frames |
| 24185-24241 | Ti Plasmid Region | 57 | Non-functional sequence from Ti plasmid of A. tumefaciens |
| 24242-24266 | Left Border | 25 | T-DNA Left Border region from Ti plasmid of |
| | | | Agrobacterium tumefaciens |

Immature embryos of maize (Zea mays L.) were aseptically removed from the developing caryopsis nine to eleven days after pollination and inoculated with Agrobacterium tumefaciens strain LBA4404 containing plasmid PHP36676, essentially as described in Zhao et al., 2001. The T-DNA region of PHP36676 was inserted into the 032218 maize event. After three to six days of embryo and Agrobacterium co-cultivation on solid culture medium with no selection, the embryos were then transferred to a medium without herbicide selection but containing carbenicillin for selection against Agrobacterium. After three to five days on this medium, embryos were then transferred to selective medium that was stimulatory to maize somatic embryogenesis and contained bialaphos for selection of cells expressing the mo-pat transgene. The medium also contained carbenicillin select against any remaining Agrobacterium. After six to eight weeks on the selective medium, healthy, growing calli that demonstrated resistance to bialaphos were identified. The putative transgenic calli were subsequently regenerated to produce TO plantlets.

PCR analysis was conducted on samples taken from the TO plantlets for the presence of a single copy cry1A.88, cry2A. 127, mo-pat and vip3Aa20 transgenes from the PHP36676 T-DNA and the absence of certain Agrobacterium binary vector backbone sequences by PCR. Plants that were determined to be single copy for the inserted genes and negative for vector backbone sequences were selected for further greenhouse propagation and trait efficacy confirmation. The TO plants with a single copy of the T-DNA and meeting the trait efficacy criteria, including 032218 maize, were advanced and crossed to inbred lines to produce seed for further testing.

### Example 2. Identification of Maize Event DP-032218-9

The real-time PCR reaction exploits the 5′ nuclease activity of the hot-start DNA polymerase. Two primers (SEQ ID NO: 2 and SEQ ID NO: 3) and one probe (SEQ ID NO: 4) anneal to the target DNA with the probe, which contains a 5′ fluorescent reporter dye and a 3′ quencher dye, sitting between the two primers. With each PCR cycle, the reporter dye is cleaved from the annealed probe by the polymerase, emitting a fluorescent signal that intensifies in each subsequent cycle. The cycle at which the emission intensity of the sample rises above the detection threshold is referred to as the C_{T} value. When no amplification occurs, the C_{T} calculated by the instrument is termed “undetermined,” and is equivalent to a CT value of 40.00 due to assay termination at 40 cycles.

Because the T-DNA is randomly inserted in plant genome, each insert/plant genomic DNA junction is unique. This information could be used for identification of the event. To detect maize event DP-032218-9, the forward primer was designed at the maize genome, the reverse primer at the insert, and the probe between the forward and reverse primers.

### Example 3: Sequence Characterization of Insert and Genomic Flanking Regions of Maize Event DP-032218-9

Maize (Zea mays L.) event DP-032218-9 (032218 maize) has been modified by the insertion of the T-DNA region from plasmid PHP36676 which contains four gene cassettes as disclosed above. Expression of the Vip3Aa20, Cry2A.127, and Cry1A.88 proteins confers resistance to certain lepidopteran insects.

Total genomic DNA was extracted from approximately 1 gram of frozen leaf tissue. The PHP36676 T-DNA insert/flanking genomic border regions were amplified by PCR. Each PCR fragment was then cloned into a commercially available plasmid vector and characterized by Sanger DNA sequencing. Individual sequence reads were assembled and manually inspected for accuracy and quality. A consensus sequence was generated by majority-rule. The resulting sequence comprising the genomic 5′ flanking sequence, inserted fragment from PHP36676, and the genomic 3′ flanking sequence is shown in SEQ ID NO: 5. The 5′ flanking genomic region has 2330 nucleotides from 1-2330 bp of SEQ ID NO: 5 and the 3′ flanking genomic region has 2123 nucleotides from 26550-28672 bp of SEQ ID NO: 5. 24 bp of Right Border were deleted and 23 bp of Left Border were deleted from the PHP36676 (SEQ ID NO: 1) insert after transformation, which is reflected in SEQ ID NO: 5.

### Example 4—Event-Specific Identification System Maize Event DP-032218-9

The event-specific PCR assay for DP-032218-9 maize was designed at the 3′ junction between the genomic DNA and the 32218 insert. The reverse primer (SEQ ID NO: 2) is situated within maize genomic DNA. The forward primer (SEQ ID NO: 3) is situated within the inserted DNA and the probe (SEQ ID NO: 4) spans the junction. Hereafter, this event-specific PCR assay for 32218 maize will be referred to as the 32218 assay.

This method event-specific real-time qualitative Taqman® PCR was developed for DNA template extracted from maize tissues, seed and grain containing both genetically modified and conventional maize for use on both a Roche LC480 Real-Time PCR system and an Applied Biosystems ViiA7 Real-Time PCR system. Table 3 shows the 32218 assay primers and resulting amplicon (SEQ ID NO: 12). Table 4 shows the preparation of the 32218 assay mix. Table 5 shows the PCR cycle profile for the 32218 assay. The resulting DP-32218-9 assay amplicon sequence (Length: 65 bp) is shown in SEQ ID NO: 12.

TABLE 3

| | |
|---|---|
| Forward Primer | AGTTGTCTAAGCGTCAATTTGTGAA (SEQ ID NO: 2) |
| Target genetic element | 3′ of insert |
| Reverse Primer | GATTTTTTGGAGCGGAATGG (SEQ ID NO: 3) |
| Target genetic element | 3′ maize host genome |
| Probe | FAM-ATTCTCCTCAGATCTGG-MGB |
| | (SEQ ID NO: 4) |
| Target genetic element | 3′ maize border region |
| Amplicon | AGTTGTCTAAGCGTCAATTTGTGAATATTCTCCTCAGATCT |
| | GGAACCATTCCGCTCCAAAAAATC |
| | (SEQ ID NO: 12) |

TABLE 4

| | Final | |
|---|---|---|
| Reagent | Concentration | μl/reaction |
| Bioline Lo-Rox ® PCR master | 1x | 5.0 |
| mix (2X) | | |
| DP-Ø32218-9-Forward primer¹ | 300 nM | 0.017 |
| DP-Ø32218-9-Reverse primer¹ | 300 nM | 0.017 |
| DP-Ø32218-9-probe¹ | 80 nM | 0.009 |
| 30% Bovine Serum Albumin² | 0.036% | 0.033 |
| HPLC molecular biology grade | N_A | 3.924 |
| water | | |
| Template DNA | N_A | 1.0 |

| | |
|---|---|
| Total Reaction Volume | 10.0 |

¹Primers to support assay mixture are at a working concentration of 200 μM; probes to support assay mixture are at a working concentration of 100 μM.
²Final concentration of 30% Bovine Serum Albumin (BSA) is based on the percent of BSA present in the final reaction volume.

TABLE 5

| Step | Temperature | Time | # Cycles |
|---|---|---|---|
| Initial | 95° C. | 20 seconds | 1 |
| Denaturation | | | |
| Denaturation | 95° C. | 1 second | 40 (ViiA7); |
| | | | 45 (LC480) |
| Annealing | 60° C. | 20 seconds | 40 (ViiA7); |
| | | | 45 (LC480) |

The maize-specific reference PCR assay used for relative quantification is a pre-validated maize-specific PCR assay (EU-RL-GMFF, 2005) for Zea mays L. High Mobility Group (HMG) Protein A gene (hmgA) (Krech et al., Gene 234: 45-501999). Hereafter this maize-specific reference assay will be referred to as the HMG assay. The HMG assay amplifies a 79 bp product based upon GenBank Accession No. AJ131373. Table 6 shows the HMG assay primers and resulting amplicon (SEQ ID NO: 16). Table 7 shows the preparation of the HGM assay mix. Table 8 shows the PCR cycle profile for the HGM assay. Table 9 shows the Genomic Controls for the assay.

TABLE 6

| | |
|---|---|
| hgmA-Forward Primer | TTGGACTAGAAATCTCGTGCTGA |
| | SEQ ID NO: 13 |
| Target genetic | hmgA maize gene |
| element | |
| hgmA-Reverse Primer | GCTACATAGGGAGCCTTGTCCT |
| | SEQ ID NO: 14 |
| Target genetic | hmgA maize gene |
| element | |
| hgmA-Probe | VIC-GCGTTTGTGTGGATTG-MGB |
| | SEQ ID NO: 15 |
| Target genetic | hmgA maize gene |
| element | |
| Amplicon | TTGGACTAGAAATCTCGTGCTGATTAATTGTTTTACGCGT |
| | GCGTTTGTGTGGATTGTAGGACAAGGCTCCCTATGTAGC |
| | SEQ ID NO: 16 |

TABLE 7

| | Final | |
|---|---|---|
| Reagent | Concentration | μl/reaction |
| Bioline Lo-Rox ® PCR master | 1x | 5.0 |
| mix (2X) | | |
| hmgA-Forward primer¹ | 300 nM | 0.017 |
| hmgA-Reverse primer¹ | 300 nM | 0.017 |
| hmgA-probe¹ | 80 nM | 0.009 |
| 30% Bovine Serum Albumin² | 0.036% | 0.033 |
| HPLC molecular biology grade | N_A | 3.924 |
| water | | |
| Template DNA | N_A | 1.0 |

| | |
|---|---|
| Total Reaction Volume | 10.0 |

¹Primers to support assay mixture are at a working concentration of 200 μM; probes to support assay mixture are at a working concentration of 100 μM.
²Final concentration of 30% Bovine Serum Albumin (BSA) is based on the percent of BSA present in the final reaction volume.

TABLE 8

| Step | Temperature | Time | # Cycles |
|---|---|---|---|
| Initial | 95° C. | 20 seconds | 1 |
| Denaturation | | | |
| Denaturation | 95° C. | 1 second | 40 (ViiA7); |
| | | | 45 (LC480) |
| Annealing | 60° C. | 20 seconds | 40 (ViiA7); |
| | | | 45 (LC480) |

TABLE 9

| Type of Control | Description | Expected Result | Interpretation |
|---|---|---|---|
| No Template Control | HPLC Water | LowDNA; no PCR | Mix is not |
| | Negative control | product | contaminated |
| Homozygous/Positive | Ground seed or | DNA samples will | Amplification is |
| DNA control | leaf derived from | score as Positive | detected for both |
| (Event DP-Ø32218-9) | known transgenic | and 2 copy, or | 6′FAM and VIC |
| | material that is | Homozygous, for | assays (DP- |
| | verified to be | the Target; | Ø32218-9 and |
| | homozygous for | Endogenous gene | hmgA) |
| | DP-Ø32218-9 | will amplify as | |
| | | expected | |
| Heterozygous/Positive | Ground seed or | DNA samples will | Amplification is |
| DNA control | leaf derived from | score as Positive | detected for both |
| (Event DP-Ø32218-9) | known transgenic | and 1 copy, or | 6′FAM and VIC |
| | material that is | Heterozygous for | assays (DP- |
| | verified to be | the Target; | Ø32218-9 and |
| | heterozygous for | Endogenous gene | hmgA) |
| | DP-Ø32218-9 | will amplify as | |
| | | expected | |
| Wild type DNA control | Ground seed or | DNA samples will | Amplification is |
| | leaf derived from | score as Negative | only detected for |
| | known wild type | and Nullizygous for | VIC assay (hmgA) |
| | material that is | the Target; | |
| | verified to be Null | Endogenous gene | |
| | for DP-Ø32218-9 | will amplify as | |
| | | expected | |

The PCR product is measured during each cycle (real-time) by means of a target-specific oligonucleotide probe labeled with two fluorescent dyes: 6-carboxyfluorescein (FAM™) was used as a reporter dye at the 5′ end of target-specific oligonucleotide probe for the event-specific 32218 maize assay and 4,7,2′-trichloro-7′-phenyl-6-carboxyfluorescein (VIC®) a reporter dye at the 5′ end of target-specific oligonucleotide probe for the HMG assay and a non-fluorescent quencher dye attached to an minor groove binding moiety (MGB) was used at the 3′ end. The 5′ nuclease activity of Taq DNA polymerase cleaves the probe and liberates the fluorescent moiety during the amplification process. The resulting increase in fluorescence during amplification is measured and recorded. For each sample, calculate the ACT by subtracting the assigned CT value of the target from the assigned CT value of the endogenous reference gene (calculated on a sample replicate basis). Generate a final composite result for each sample from the data supporting all sample replicates using the defined scoring criteria, as shown in Table 10, which includes a ACT threshold of −5 and a CT threshold of 35. All technical replicates should produce consistent CT values for both assays, with exception to the Null genomic control and the No Template Control, as described in the Genomic Controls Table.

TABLE 10

| | ΔC_{T} | C_{T} value for | C_{T} value for |
|---|---|---|---|
| Score | Value | endogenous gene | target |
| Positive | >−5 | <35 | <35 |
| Negative | <>−5 | <35 | >35 |
| Undetermined | <−5 | <35 | <35 |
| Low DNA | — | >35 | — |

The real-time PCR method was optimized and validated using both a Roche LC480 Real-Time PCR system and an Applied Biosystems ViiA7 Real-Time PCR system. The method can also be applied on a different platform however, with minimal optimization and adaptation.

### Example 5—Zygosity Duplex Assay for Maize Event DP-032218-9

To determine zygosity of the 32218 event real-time PCR was performed essentially as described in Example 4 using the reaction mix as shown in Table 11.

TABLE 11

| | Final | |
|---|---|---|
| Reagent | Concentration | μl/reaction |
| Bioline Lo-Rox ® PCR master | 1x | 5.0 |
| mix (2X) | | |
| DP-Ø32218-9-Forward primer¹ | 600 | 0.033 |
| DP-Ø32218-9-Reverse primer¹ | 600 | 0.033 |
| DP-Ø32218-9-probe¹ | 80 | 0.033 |
| hmgA-Forward primer¹ | 600 | 0.033 |
| hmgA-Reverse primer¹ | 600 | 0.009 |
| hmgA-probe¹ | 80 | 0.009 |
| 30% Bovine Serum Albumin² | 0.036% | 0.033 |
| HPLC molecular biology grade | N_A | 3.817 |
| water | | |
| Template DNA | N_A | 1.0 |

| | |
|---|---|
| Total Reaction Volume | 10.0 |

¹Primers to support assay mixture are at a working concentration of 200 μM; probes to support assay mixture are at a working concentration of 100 μM.
²Final concentration of 30% Bovine Serum Albumin (BSA) is based on the percent of BSA present in the final reaction volume.

For the duplex reaction cited above, the assay has been optimized so that the efficiencies of both the target and the endogenous genes are approximately equivalent, allowing the 2^{-ΔΔC}_{T} method to be used to calculate copy number, or zygosity. The calibrator in this analysis is the heterozygous, or 1 copy genomic control where amplification of the target is normalized to the endogenous reference and relative to the 1 copy calibrator. For the 1 copy calibrator, the ΔΔC_{T} equals zero and 20 equals 1, representing the single copy of the calibrator. Calculations for zygosity of the raw data using the 2^{-ΔΔC}_{T} method are as follows:
Calculate the average C_{T} for each sample set of technical replicates and genomic control/calibrator replicates for both the target and the endogenous genes from the raw data
Calculate the ΔC_{T} for the sample by subtracting the Average Endogenous C_{T}-Average Target C_{T}
Calculate the ΔΔC_{T} for the sample by subtracting the Average ΔC_{T} Calibrator —Average ΔC_{T} Sample
Normalize the sample by taking ΔΔC_{T} value and applying the value to 2^{-ΔΔC}_{T} formula
The acceptable range of each zygosity population, is estimated by incorporation of ΔΔC_{T} plus the standard deviation and ΔΔC_{T} minus the standard deviation
Populations for 2 copy number, or homozygous populations, can be estimated based on the following rules:
   Acceptable differential between 1 copy/heterozygous and 2 copies/homozygous:
      ΔΔC_{T} should fall between 0.7 and 1.3

Having illustrated and described the principles of the present disclosure, it should be apparent to persons skilled in the art that the disclosure can be modified in arrangement and detail without departing from such principles. We claim all modifications that are within the spirit and scope of the appended claims.

All publications and published patent documents cited in this specification are incorporated herein by reference to the same extent as if each individual publication or patent application was specifically and individually indicated to be incorporated by reference.

1. A method of detecting in a sample comprising corn nucleic acids the presence of a nucleic acid molecule unique to event DP-032218-9, the method comprising:
(a) contacting the sample with a first polynucleotide primer of SEQ ID NO: 2 and a second polynucleotide primer of SEQ ID NO: 3;
(b) performing a nucleic acid amplification reaction, thereby producing an amplicon of SEQ ID NO: 12; and
(c) detecting the amplicon using an DP-032218-9 event specific probe of SEQ ID NO: 4.

2. The method of claim 1, wherein the method further comprises contacting the sample with a first polynucleotide primer unique to maize HMG of SEQ ID NO: 13 and a second polynucleotide primer unique to maize HMG of SEQ ID NO: 14; and detecting the amplicon of SEQ ID NO: 16 with a maize HGM specific probe of SEQ ID NO: 15, wherein the HGM amplification serves to determine the quality of the nucleic acid in the sample.

3. The method of claim 1, wherein the amplification method is a real-time PCR method.

4. The method of claim 1, wherein the real-time PCR is performed in thermo-cycler instrument.

5. The method of claim 1, wherein the probe is attached to a conventional detectable label, reporter molecule, and/or quencher molecule.

6. The method of claim 5, wherein the detectable label is a fluorescent dye.

7. The method of claim 6, wherein the fluorescent dye is selected from FAM™ and VIC®.

8. The method of claim 5, wherein the quencher molecule is a non-fluorescent quencher dye attached to a minor groove binding moiety.

9. A kit for detecting nucleic acids that are unique to event DP-032218-9 comprising the nucleic acid molecules of SEQ ID NO: 2, SEQ ID NO: 3 and SEQ ID NO: 4.

10. A kit for detecting nucleic acids that are unique to event DP-032218-9 comprising SEQ ID NO: 12.

11. A method for determining in a sample comprising corn nucleic acids the zygosity of a nucleic acid unique to event DP-032218-9, the method comprising:
(a) contacting the sample with a first polynucleotide primer unique to event DP-032218-9 of SEQ ID NO: 2 and a second polynucleotide primer unique to event DP-032218-9 of SEQ ID NO: 3;
(b) contacting the sample with a first polynucleotide primer unique to maize HMG of SEQ ID NO: 13 and a second polynucleotide primer unique to maize HMG of SEQ ID NO: 14;
(c) performing a nucleic acid amplification reaction, thereby producing an amplicon unique to detecting the amplicon of SEQ ID NO: 12 and an amplicon unique to maize HMG of SEQ ID NO: 16;
(d) detecting the amplicon of SEQ ID NO: 12 with tan event DP-032218-9 specific probe of SEQ ID NO: 4;
(e) detecting the amplicon of SEQ ID NO: 16 with a maize HMG specific probe of SEQ ID NO: 15; and
(f) determining the zygosity of the DP-032218-9 event.

12. The method of claim 11, wherein the amplification method is a real-time PCR method.

13. The method of claim 11, wherein the real-time PCR is performed in thermo-cycler instrument.

14. The method of claim 11, wherein the probes are attached to a conventional detectable label, reporter molecule and/or quencher molecule.

15. The method of claim 14, wherein the detectable label is a fluorescent dye.

16. The method of claim 15, wherein the fluorescent dye is selected from FAM™ and VIC®.

17. The method of claim 14, wherein the quencher molecule is a non-fluorescent quencher dye attached to a minor groove binding moiety.

18. The method of claim 11, wherein the zygosity is determined by the 2^{-ΔΔC}_{T} method.

19. A kit for determining in a sample comprising corn nucleic acids the zygosity of a nucleic acid unique to event DP-032218-9 comprising SEQ ID NO: 2, SEQ ID NO: 3, SEQ ID NO: 4, SEQ ID NO: 13, SEQ ID NO: 14 and SEQ ID NO: 15.


SEQUENCE LISTING (16 sequences)

SEQ ID NO: 1 - DNA, 24266 bp, Artificial Sequence
  32218-9 event T-DNA
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccaccggtggtaccgagctcgtttaaacgctcttcaactggaagagcggttaccagagctggtcacctttgtccaccaagatggaactggcgcgcctcattaattaagtcagcggccgctctagttgaagacacgttcatgtcttcatcgtaagaagacactcagtagtcttcggccagaatggccatctggattcagcaggcctagaaggccatttatctatcaactttgtataataaagttgcccggtccttaggcggaccgggccatctaggccgcggccgcactgtcaagctattattagcttctttaataagtccaatgtgaacaaaccgtctagggttagatggattgctttcacagatttccttactggtctaggaatccctgtaaatatagagcacatagatggaaaaaataaccatctggctgatgctctgtccagattagtaactggttttgtttttgcagaaccacaatgtcaagacaagttccaggacgatttagggaaattggaagcagctcttcaggagaagaaagaggctccgcaagcaatgcacgtagaatatgtctccctgttgatcagatcagcggaccgcattacccgctcgctctgctttatgagggactcgtctcacagcagaatttactcatgcaggccaggcaaagaaccaatgaaggccttaatctgcgaacagaagtcatgccaatccaaaggcgacttagggaatacgaggactgtgcactccaagagtgcattcaatcagcaagacaactggtggccctccaccagcacaaactcgcttacatcagaagcaaagctacaagggacaacgcatatgccgataggctacccacatgcaatcgggaccacgagcaactgtgtgaagtggtcgagctattagaaggaatctcggaaagaatcagcgatacagctgtctaggacagctggcttcaattatggagcgtgatggacccccccgcaataatccaaagtttggtgtgcttttagtagtgcgtctttatggaccactactttattgtaataatcgatgctttttgtagtgcgctcttcgtgcgctctactttatgcttttgcttttgtaagtgcgctgtaagtgcgcctgtctttcttcagatgcttatcctttaagcatcttttgctttttgcgtggcatcctttagttcacaatttaaagaatgacgatggggcccaagatgtgcacccggttctctaaattgcctatataaggatatgccatagccttgtttttgcaagtcaggaatacctgagcataacttggctaagcaaaagtttgtaagtgttctaagctttcatttgtaaactttctgtttggttttaataaaatctctcgtcaatcgttgtgaacatatattgtttgtttgtattgttgtatcttatttgttgtggtgataaggatcttcgatatcccggactggcgccaggtccgccttgtttctcctctgtctcttgatctgactaatcttggtttatgattcgttgagtaattttggggaaagcttcgtccacagtttttttttcgatgaacagtgccgcagtggcgctgatcttgtatgctatcctgcaatcgtggtgaacttatttcttttatatccttcactcccatgaaaaggctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggcgccttgggatccatggctgcgaccactctcacgagcgctctcccaggagcctttagcagctctcagagaccttcggctccgttcaacctccagaggagccctagagtcctcagacgcttcaaccgcaagaccggtagacagccacgcggtctcgtcagagctgctaaggctcagcgctctggtaccagatccatgggcaactccgttctcaattccggaaggactacgatctgtgatgcgtacaacgttgcagctcatgatccgttctcattccagcacaagtcacttgacactgttcagagggagtggactgagtggaagaagaacaaccattcgctgtatctcgatccgatcgttggaactgtggcttcattcctgctcaagaaggtcggttctctcgttggtaagaggattctctcggaactcaggaacttgatcttcccatctggtagcacaaacctcatgcaggacatacttagggaaactgagcagttcctgaaccaacgccttgacactgataccttggcaagggtcaatgctgagttgacaggtcttcaagcgaacgttgaggagttcaatcgccaagttgacaacttccttaaccctaaccggaatgccgttcctctgtctatcacgtcatctgtcaacacgatgcagcagctgttcttgaaccggcttcctcaattccagatgcaaggttaccaactgttgctccttccactgttcgctcaagctgctaatctgcatctgagcttcatcagggatgtcatcctgaatgccgacgaatggggtatatctgcagctacacttcgcacttacagggactacctgaagaactacacgcgcgactactcgaactactgcatcaacacctatcagtccgccttcaaaggcctgaacacgaggcttcatggtacgttggagtttcggacgtacatgttcctgaacgtgttcgagtatgtctccatctggtcactcttcaagtaccagtcattgctggtctcgtcaggtgctaacctgtacgcatcaggatcaggacctcaacagacgcaatcgttcacgtctcaagactggccattcctgtatagcttgttccaagtcaactccaactacgtgctgaacggcttctctggtgctaggttgtccaacactttcccaaacatcggtggacttccaggaagcactacgactcatgcactgcttgctgcaagggtcaactactctggaggtatctcatctggtgacattggagcttcaccgttcaaccagaacttcaactgcagcacattccttccacctttgcttacgccattcgttagatcatggcttgactctggatctgatagggaaggagtcgctactgtgaccaactggcagacagagtcattcgagacaacactcggtcttcgctcaggagcattcacagcaagaggcaacagcaactacttcccagactacttcattcgcaacatctctggagttcctcttgtcgttaggaacgaggaccttcgcagacctctgcactacaatgagatcaggaacattgcctcaccttcaggtacacctggtggagcaagggcttacatggtctcagttcacaaccgcaagaacaacatccatgcagttcatgagaacggatcgatgatccacttggcacctaacgactacactggattcacgatctcacctatccatgctactcaggtgaacaaccagactcgcactttcatcagcgagaagttcggcaaccaaggcgattctctgaggtttgagcagaacaacacgactgcaaggtacactctcagaggtaacggcaactcgtacaacctgtacttgcgcgtctccagcataggcaactcaacgatccgcgttaccatcaacggtcgcgtttacactgctacaaacgtcaacacgaccactaacaacgatggtgtcaacgacaatggtgctcgcttcagcgacatcaacatcggtaacgttgtcgcaagcagcaactctgacgttcctctggacatcaacgttacgttcaactctggaacacagttcgatttgatgaacaccatgctggttccgacgaacatcagcccattgtactgagttgcgtggaccgaagcttgcgcgcctaggtttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaaagcttgaaaagaaaatgcagccaagcttaccaagtaagttatgtgtattaaccagaggaagagagaatcttgcaaaatttcaacaaacacaaaaagaagtattactacgattggtggagaaagaaaacgattccaaatcttgaactgttgttgtaaaagcatagcagaaagtgggagacaaccgaaatagaaatgactataacttaatttaatgttatcattataatttcttctagcaaatatttagaaagtaaatatcacatcaacctttaatgtaattaagctttctctttttgattcatgtgagatgaaaagaaaaaaaagaagagaaaagtgtagaaaacacatcatttctaagctgaaggtacatagtacccttgtacttttggtttcacctgcatagagaaaacccacaagaatatgacagtctgatttgtcagtctcattctcaagcaacatttctctatccgttactttcatggtgaataacacaatccatcatcaatactttgtgttactcagaaactgaaagttattccgagtcttgcatatctttgggcctactcgtttttctaccattattgctgattgttaagctctcgctacttgaatcggcattgttggagtgggaaggttcaaaaaattggagttatgactagttgtctctttctatgtacgatggagaaaatgaataaacaactgagaaaatggctcttgtttagttgatgatgctcttaagctttccactggttgccatatatgatttgggcatttcactttgatcttaatgggccttgtaaggcccaagactcatgattatctttagttgatgctcttaattaggtgtgggcaaataattcaaactgtatgtacccgaccaaaaccaaagcaaaaataatcgaaccaaaccgaaaatttaaaaataaccgaatgaaaactaaatcctataactgaaagaactgaaaccgaatcaaaatatttaatgtaaccaaaaatatccgaaatataattatattgtcaaaaatattaataatttctagattaaataattaaaaatacttaaaaatttatataaaatagtaaaaatactcgaaaataaccacaaatattcaaaaacaaccgaaatatcccaaaatattcaaagcaaaataaccgaatggataccaaattttaaaaccgaaaaaactggaacaaaaccagaatcgaaccaaaatttcaaaaatcgaataaatactaaactttagaacaaaaaaaaacgataaccgaatgtatacgaaccaaagccgaattagataaccgaacgtccaggactactcttaatctttccgccacttatgatttgggctattactttgtttataatgagccttttcaagctcaagttcatgattgtccgtgagatgagaaactgacttgttggattcgaaaccctagctagtattggttaatacttaatacataaatgacctgcattgacatcatcatccaagaaaataaaaattgtatgcttgagatatttagttttcctagctaggttttctttattttagtaccgaatctttaggtgtgccacgttaatttagacccattttttcatacttaccaactgagtctagtttaatcatgactataatcgtataaaatgattcagtcgacgtcattgcgaacgtatataaaatcatccaaattgacgtcattccaaagaggtaagcatgcttatctaagagtccgagcatactaaacaagacgacattttatttgcactctaaatcaaattttgtattgcctaaagaaaaacaatcaaactcaagtttcttaaaattaatttcattcaaactaatcactttcaatatctcacatattattcatgccatttctatttgtctaaacatgatttaaaaaaaaagtaaaatacaaagattactatgcaaaaactctataaaaaaaaattcaaatttcttatttatttgtgacatcaaattttcaaaataatttttttaattatcggttgatccggtcagtcgataaaaacataaactttcagcgaccgttaaaactttcctactaccgatttagagaaaatcttagcttgaaacgtaattgtaacctgccttcatgcaagtcgcaagatatgtcatcctaagttgtatatgttttctcaaaagatgtatttacttgagaaaatacgtttcaacgttgatggacaaccaattaagaatcaagcacctttcgtaatcaatttaggcttatcgtctaaggtatactgatttacgacagttgactagacttataaggaacaaaataatagaataatttcgtcaagaaaaattgattttggactcatactttacataatattttactcttaaatttatttaagtggctcctcgcatgatcccaaagagcaagcctagactatatggaaaagtttctaaacacttcacctaatcatagagactaagatggtaattcgtaaacgacaaagcctagtgacactgtccattgtaaaattccacatcatattagtattaaacatatacatgtagtttcctgaacacatgtagtatcaaacacacttcgtggcttcttcctcgaaatcgaggcctaggcttaaggtttaaacagcccgggcgcgcccggaccgggccatctaggccccttagggagctctcgcgacgtcaatcgagtacgtacgtaagggcgacaccccctaattagcccgggtctagagtcgacagatctccatggatccgttaacggccactttgtacaagaaagctgggtgcccgggaataagtgactagggtcacgtgaccctagtcacttaggtgaccaagcttcggccgcaggatagaggacatcctggacctactgaacgtcagcaatgacgactgaaagattcccaggacaccggcggaagtggtggacccagtctaggtgcgatgcttagtcgcgcacgatgactatgtcggaaggcatctttgctttcggcaaactttagtaatactttaaggaaagtattgtacaagttaggtgcagagacaataatgcacccagctttagctttgtttatggaattattgtgtcggttgcattattggatgcctgcgtgcaccctaagcaatccccggccctcttctctataagaggagcccttgcaatcagttgcaagcatgcaagtttcccactgcaagcttacttctgagtttgagttcaagttcaataaaattcaagctttcctcttacattctgttcttgaaaggttcgatctaatcgagcgagtagagaacaagatcttttgggatttccgccgttccggatcttcgatatcccggactggcgccaggtccgccttgtttctcctctgtctcttgatctgactaatcttggtttatgattcgttgagtaattttggggaaagcttcgtccacagtttttttttcgatgaacagtgccgcagtggcgctgatcttgtatgctatcctgcaatcgtggtgaacttatttcttttatatccttcactcccatgaaaaggctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggcgccttgggatccatgggccacaacaacccgaacatcaacgagtgcatcccgtacaactgcctgtccaacccggaggtggaggtgcttggaggcgagagaatcgagaccggctacactcccatcgacatcagcctcagccttacccagttcctgctctcggagttcgtgccaggagcaggtttcgtgctgggactggtcgacgtgatctggggcatcttcggtccgtcccaatgggatgcgttcctggttcagatcgagcagctgatcaaccagcgcatcgaggagttcgccaggaaccaggccatctctagggtcgagggcctcagcaacctgtaccagatctacgcagagtccttcagagagtgggaggccgatccgaccaatccagcgctcaaggaggagatgcgcacgcagttcaacgacatgaactccgctctgacgacagccattccgctgtttgcggtccagaactaccaggtgccgctgcttagcgtgtacgtccaggctgctaacctccacctgtcggttcttcgggacgtgtcagtgttcggccagaggtggggattcgacgctgcgacgatcaactcgcgctacaacgacctcaccaggctcatcgggaactacacagaccacgcagtgcgctggcacaacaccgggttggagcggatatggggcccggactcgagagattggattcggtacaaccagttccgccgcgagctgaccctcacggtgctggacatcgtgtcgctgttcccgaactacgactcgcgcacgtacccgatccgcacggcgagccaactgaccagggagatctacaccaacccggttctcgagaacttcgacggcagctttcgcggaagcgcgcaaggcatcgaaggttcgatccgctcgccgcacctgatggacatactcaacagcatcaccatctacacggacgcgcacagaggcgagtactactggagcggacaccagatcatggcgagccctgtcggcttctctggaccagagttcacattcccgctgtacggcacgatgggtaacgctgctccgcaacagaggatcgttgctcagctcggccaaggcgtctacagaaccctgtcctcgactctgtaccggaggccgttcaacatcggcatcaacaaccagcagctttccgtccttgacggtacggagttcgcgtatggcacctcatccaacctgccttccgccgtttaccggaagtccgggacagtggacagcctcgacgagatcccgccgcagaacaacaacgtgcctccaaggcaaggcttctctcacaggctctcacacgtgtcgatgttccgctctgggttcagcaactcctccgtctccatcatccgcgctcccatgttctcgtggattcacaggagcgccgagttcaacaacacgatcgacccggagcgcatcaaccagatcccgctgaccaagagcacgaacctcggctcaggcacctctgtggtcaaaggacccggtttcactggcggcgacatcttgaggaggacaagcccagggcagatctccacgcttcgcgtcaacatcacagctccgctgtcccagcgctaccgcgttcggatcaggtacgcctcgacgaccaacctccaattccacacctcgatcgatgggaggccgatcaaccagggcaacttctccgcgacaatgtcctccggcagcaacttgcagagcggttccttccgcaccgtgggcttcaccacgccgttcaacttcagcaacgggtcctctgtcttcaccctgtcggcacatgtgttcaacagcgggaacgaggtctacatcgaccgcatcgagtttgtgccagccgaggttacgtttgaagcggagtacgacctggagcgcgcgcagaaagtggtcaacgcgctgttcacgtcctcgaaccagatcgggctcaagaccgacgtgacggactaccacatcgaccaggtgtccaacctcgtggactgcctgtccgacgagttctgcctcgacgagaagcgcgaactgtccgagaaggtgaagcacgcgaagcggctgtctgacgagcggaaccttctgcaagacccgaacttcagaggtatcaacaggcaacctgaccgcgggtggcgcggatcgacggacatcacgatccagggcggcgacgacgtgttcaaggagaactacgttacactgcccggcacagtggacgagtgttacccgacctacctgtaccagaagatcgacgagtcgaagctcaaggcgtacacgaggtacgagcttcgcggctacatcgaggactcgcaagacctggagatctacctgatccgctacaacgccaagcacgagatcgtgaacgtgcctggtactggttcactgtggccactgagcgcgcaaagcccgattgggaagtgcggtgaacccaacaggtgcgctcctcacctggaatggaatccggacctggattgttcttgccgcgatggcgagaaatgcgcgcaccactcccaccacttcaccctggacatcgacgtcggttgcaccgatctcaacgaggacttgggcgtgtgggtgatcttcaagatcaagacccaggatgggcacgccaggctcggcaacctggagttcctggaggagaagcctctgcttggtgaagcgcttgccagagtcaagagggcggagaagaagtggcgcgacaagcgcgagaagctccagctggagacgaacatcgtctacaaggaggccaaggagtccgtcgacgccctctttgtgaacagccagtacgaccggctccaggtggacacgaacatcgccatgatccatgcagccgacaagcgggttcacaggatcagggaggcttatcttccggagctgagcgtcataccgggcgtgaacgctgcgatcttcgaggagcttgagggccggatcttcacggcttacagcctctacgacgcgaggaacgtgatcaagaacggcgacttcaacaacggcctgctctgctggaacgtcaagggccacgttgacgtcgaggagcagaacaatcaccggagcgtgctggtgatccctgagtgggaagccgaggtgtctcaggaggtcagggtctgtcctggacgcggatacatccttcgcgtcacagcctacaaggagggctatggcgagggctgcgtcaccattcacgagatcgaggacaacaccgacgagctgaagttcagcaattgcgtcgaggaggaggtgtacccgaacaacaccgtcacctgcaacaactacacgggcacacaggaggagtatgagggcacctacacctctcgcaaccagggctacgatgaagcgtacggcaacaacccatcagttcccgccgactacgcctccgtctacgaggagaagtcgtacaccgacggcagacgcgagaatccttgtgagtccaacagaggctacggcgactacacgccactgccggctggatatgtgaccaaggacctggagtacttcccggagaccgacaaggtgtggatcgagatcggcgagaccgagggaaccttcatcgtcgacagcgtcgagctgctcctgatggaggagtaggttaattcgattactagtgtttttctcagacagttttctaaaaaaagggcgtttctggggaagttcgagatggttcgtaaggtgttactggctcctgtgaaccaatacatgatactgccatgataagggttataattagtcaagcagagtaagaagaaacaacagtagcagtgactccgattcctgaagatgagtcatatttgtcttgtgctcctgctgtatgaaatggatcgcatgtgtatattcgtcgccgcgccgcactggtgtaacctgttgcctcagagtttgcttttagctggttctgttttaaaaataagtactgttttttggttggctgcaagccattctgaacttcagtttaccaattgtttttatgttgtggttgaatattttaattttttatttaatgtttggttctttttttatatatatttgcaaaaatgatacaagtggtcaagttttcatatagtatgggctctatttcctagagctctacctctaggaacgaattttgtggaggttttcttttggctagttaggcaaagtccccatatcttgcaggctaaatcaagaagaagctctgtcaaacagttttttttactgaaaagtgattaaagagtagtttctcctagatcacttcagagtttatcctagagaatcatgggaatcaaattcagttagaggatcatttcttacaaagaatcaactttcgtagagaatctaaagcagaaagagctttgacaaacttacccttagagcaattccaacattctcgcgtgagtttcttcgcgccgttgttttgcggtgacttcatctggacgtcccgcgacatagagacgcttgtattgatcatgagagcttgtgtggtcatacacaatataattgttaaagatgaaagagatgtggaccttaatgagcgattcgactttgatggtgaaaatgtgcaaccttctcatggtatttctactcgcacactagctgaatttattgaagctcataaaaagatccgagacaaagaaatacattttcaattgaaagaagacctaatcaagcacttatgggaattcctaggcttaaggtttaaacagccccctccggcggtgtcccccactgaagaaactatgtgctgtagtatagccgctggctagctagctagttgagtcatttagcggcgatgattgagtaataatgtgtcacgcatcaccatgcatgggtggcagtctcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacgaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtcaacggaagcgctgcagaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagagaattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtgaccaagcttggcgcgccgtcccattctggccgaatttaagtgactagggtcacgtgaccctagtcacttaccggattctggccggagcctgcttttttgtacaaacttgaagctggccttctaggcccggaccgggtgaccaagcttgggccgcgtttaaacttcgaaacgcgtggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatgaacaagaacaacaccaagctgagcacccgcgccctgccgagcttcatcgactacttcaacggcatctacggcttcgccaccggcatcaaggacatcatgaacatgatcttcaagaccgacaccggcggcgacctgaccctggacgagatcctgaagaaccagcagctgctgaacgacatcagcggcaagctggacggcgtgaacggcagcctgaacgacctgatcgcccagggcaacctgaacaccgagctgagcaaggagatccttaagatcgccaacgagcagaaccaggtgctgaacgacgtgaacaacaagctggacgccatcaacaccatgctgcgcgtgtacctgccgaagatcaccagcatgctgagcgacgtgattaagcagaactacgccctgagcctgcagatcgagtacctgagcaagcagctgcaggagatcagcgacaagctggacatcatcaacgtgaacgtcctgatcaacagcaccctgaccgagatcaccccggcctaccagcgcatcaagtacgtgaacgagaagttcgaagagctgaccttcgccaccgagaccagcagcaaggtgaagaaggacggcagcccggccgacatcctggacgagctgaccgagctgaccgagctggcgaagagcgtgaccaagaacgacgtggacggcttcgagttctacctgaacaccttccacgacgtgatggtgggcaacaacctgttcggccgcagcgccctgaagaccgccagcgagctgatcaccaaggagaacgtgaagaccagcggcagcgaggtgggcaacgtgtacaacttcctgatcgtgctgaccgccctgcaggcccaggccttcctgaccctgaccacctgtcgcaagctgctgggcctggccgacatcgactacaccagcatcatgaacgagcacttgaacaaggagaaggaggagttccgcgtgaacatcctgccgaccctgagcaacaccttcagcaacccgaactacgccaaggtgaagggcagcgacgaggacgccaagatgatcgtggaggctaagccgggccacgcgttgatcggcttcgagatcagcaacgacagcatcaccgtgctgaaggtgtacgaggccaagctgaagcagaactaccaggtggacaaggacagcttgagcgaggtgatctacggcgacatggacaagctgctgtgtccggaccagagcgagcaaatctactacaccaacaacatcgtgttcccgaacgagtacgtgatcaccaagatcgacttcaccaagaagatgaagaccctgcgctacgaggtgaccgccaacttctacgacagcagcaccggcgagatcgacctgaacaagaagaaggtggagagcagcgaggccgagtaccgcaccctgagcgcgaacgacgacggcgtctacatgccactgggcgtgatcagcgagaccttcctgaccccgatcaacggctttggcctgcaggccgacgagaacagccgcctgatcaccctgacctgtaagagctacctgcgcgagctgctgctagccaccgacctgagcaacaaggagaccaagctgatcgtgccaccgagcggcttcatcagcaacatcgtggagaacggcagcatcgaggaggacaacctggagccgtggaaggccaacaacaagaacgcctacgtcgaccacaccggcggcgtgaacggcaccaaggccctgtacgtgcacaaggacggcggcatcagccagttcatcggcgacaagctgaagccgaagaccgagtacgtgatccagtacaccgtgaagggcaagccatcgattcacctgaaggacgagaacaccggctacatccactacgaggacaccaacaacaacctggaggactaccagaccatcaacaagcgcttcaccaccggcaccgacctgaagggcgtgtacctgatcctgaagagccagaacggcgacgaggcctggggcgacaacttcatcatcctggagatcagcccgagcgagaagctgctgagcccggagctgatcaacaccaacaactggaccagcaccggcagcaccaacatcagcggcaacaccctgaccctgtaccagggcggcaggggcatcctgaagcagaacctgcagctggacagcttcagcacctaccgcgtgtacttcagcgtgagcggcgacgccaacgtgcgcatccgcaactcccgcgaggtgctgttcgagaagaggtacatgagcggcgccaaggacgtgagcgagatgttcaccaccaagttcgagaaggacaacttctacatcgagctgagccagggcaacaacctgtacggcggcccgatcgtgcacttctacgacgtgagcatcaagtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgacctcgaggcctaggcttaaggtttaaacagcccgggcgcgccggtaccgagctcgaattcggtaacccggtccgggccattctggccgtaccgagctcgaattcggcccaacttttctatacaaagttgatagcgataaatcctgaggatctggtcttcctaaggacccgggatatcggaccgattaaactttaattcggtccgataacttcgtatagcatacattatacgaagttatacctggtggcgccgctagcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcaattcgctagcgaagttcctattccgaagttcctattctctagaaagtataggaacttcagatccaccgggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccctagcgtatacgaagttcctattccgaagttcctattctccagaaagtataggaacttctgtacacctgagctgattccgatgacttcgtaggttcctagctcaagccgctcgtgtccaagcgtcacttacgattagctaatgattacggcatctaggaccgactagctaactaactagtacgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 2 - DNA, 25 bp, Artificial Sequence
  32218-9 event PCR forward primer
agttgtctaagcgtcaatttgtgaa

SEQ ID NO: 3 - DNA, 20 bp, Artificial Sequence
  32218-9 event PCR reverse primer
gattttttggagcggaatgg

SEQ ID NO: 4 - DNA, 17 bp, Artificial Sequence
  32218-9 event PCR probe
attctcctcagatctgg

SEQ ID NO: 5 - DNA, 28672 bp, Artificial Sequence
  32218-9 event with 5′ and 3′ flanking sequences
caaccgaaagtgctattgtacaaggtaaaaggattgaacatactgacaaaatctgaaactaactttaaccaacatgtagtgctgtttccttttttacaaacacaaataaccaacagcatcttaaataaagtagcagcaactgtcaagaaatcaagaccttggaagctcgatgcagatgaactctacaccaagcagtcaaaccatttatttaagtccaaaaacattccagagttgcagtaacttgaacaataagcagtcagaatctgaaagtaacttcaaccaacaagacgtatatggcatgcggatcattcaagacacacacccttggcaactgccaaaagtcagcgcatgttgcctacggaaagctacttgccgtgggagaagtttgtttcgtactattacacgcgttattaatggtataatgtatgtcgttatacatgtagcatgcactaattaagttgagatctaaaccttgtgctaccgaaaacccattgagatcaaaataccaagttccatgagtgggaatcatattgagatgagattaaaaaaaaaattctagctcgtccgagtccgactgaacagatcactagatccctccggcggcaaagagaaaaagaaaccccggcaggcgccaggcccgatccaaccaattggtaagcgggtagccaactcacacatgcagctaaagcatgcaacggcagcaaatccatgacacagacagaaggcagcacgcagcagggaaggtggggaagagaccttggtgtcgagcatgacggcgcagaggatgccggtgttgtgcatggcctggcggaggctgtcgagcgtctcctggtggtactggtgggtgccgtgggagaagttgaagcgcgcgacgttcatgccggcgcggagcagcttctcgagcatgggcacggagcgggaggccgggccaagcgtgcagacgagcttggtcttgggcagccgcgcgtcgccggcgccgcggtccaggtccgccaggatcttcgccatgtcgatgttcgccatcgcgcggcgctccggatctcgcctctgtctgctccgcggtccggggcacggggaatgggggagggagaggtgtcggcgtcggcgtcggcgtcgagcggcggagagcagcgagatgggattcgagatttggtggcgaagtttccctttatagaaaggagaaggggggtgggagtggggaattgcgattccgtgggcgtctctgggctgggcgaggacgacgagcggagaccggccggagatggaaggcgcatgcaccaggcaggggtcagtcgctggacacgtggtgcccactgaatttgggatagtaccttggagtgggagcgagagcgatggtcgcaggaaagagcatggcgggcctacccgtcagtgacccctgcgtccacctgactgttagggcatgtacagtggtgtttaatgtggagtctcttaagatgtttaagggggttatttgcaaaaaacatcttagagccgtctctccgtgaagagacgcctctggctcgtaaaccaagtagcaacagacgcctcacttcccactgtacgaatttgtcgtctgttctatcgatctgatgctatacaaatacatttaattctgtatttattaatagactacgtttatagacactccattgtacaatagagtctcttagttgtctcctgtgcttggagaaccgttttggtgtctctccactgtacatgcccttatgccgatgtgtcgttgttagtcaggcctgtctgaggtttccctgcactgcaacggtgaaagggtcttcttttgcgacggcaattgagaaagaataataattataatatctaaattagatctgaaattagtgctgttgaagtggtgggatttgcccgccttttcgaatttcgatgcgagcgcgggtgatggtggggtggatgattcctgggcatttgccaaaaccagcgtcgatcgtcagaaaccaggccctccgttttggatgctttcggttcgtgttcctcgacagcgttcgttgtcggcggctctgcgtctgcccgtttctttcgtcacggtcgttttcatctctcgtttttccctggcattctcccattccacaacaaaaggcacactaatcttctgaacgtttcctgttgctgttagcaacggttctaattatttgagcctcaacggctggaccagaccatttctcgtcaaaatatttatacgctcgtcacatcgagaatgtggacagatggagctacgaaaatatacgttctctccgtgctaaaataaattttaaattttgcatataaatattcaatcttctccgtcttaatatgaatttaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccaccggtggtaccgagctcgtttaaacgctcttcaactggaagagcggttaccagagctggtcacctttgtccaccaagatggaactggcgcgcctcattaattaagtcagcggccgctctagttgaagacacgttcatgtcttcatcgtaagaagacactcagtagtcttcggccagaatggccatctggattcagcaggcctagaaggccatttatctatcaactttgtataataaagttgcccggtccttaggcggaccgggccatctaggccgcggccgcactgtcaagctattattagcttctttaataagtccaatgtgaacaaaccgtctagggttagatggattgctttcacagatttccttactggtctaggaatccctgtaaatatagagcacatagatggaaaaaataaccatctggctgatgctctgtccagattagtaactggttttgtttttgcagaaccacaatgtcaagacaagttccaggacgatttagggaaattggaagcagctcttcaggagaagaaagaggctccgcaagcaatgcacgtagaatatgtctccctgttgatcagatcagcggaccgcattacccgctcgctctgctttatgagggactcgtctcacagcagaatttactcatgcaggccaggcaaagaaccaatgaaggccttaatctgcgaacagaagtcatgccaatccaaaggcgacttagggaatacgaggactgtgcactccaagagtgcattcaatcagcaagacaactggtggccctccaccagcacaaactcgcttacatcagaagcaaagctacaagggacaacgcatatgccgataggctacccacatgcaatcgggaccacgagcaactgtgtgaagtggtcgagctattagaaggaatctcggaaagaatcagcgatacagctgtctaggacagctggcttcaattatggagcgtgatggacccccccgcaataatccaaagtttggtgtgcttttagtagtgcgtctttatggaccactactttattgtaataatcgatgctttttgtagtgcgctcttcgtgcgctctactttatgcttttgcttttgtaagtgcgctgtaagtgcgcctgtctttcttcagatgcttatcctttaagcatcttttgctttttgcgtggcatcctttagttcacaatttaaagaatgacgatggggcccaagatgtgcacccggttctctaaattgcctatataaggatatgccatagccttgtttttgcaagtcaggaatacctgagcataacttggctaagcaaaagtttgtaagtgttctaagctttcatttgtaaactttctgtttggttttaataaaatctctcgtcaatcgttgtgaacatatattgtttgtttgtattgttgtatcttatttgttgtggtgataaggatcttcgatatcccggactggcgccaggtccgccttgtttctcctctgtctcttgatctgactaatcttggtttatgattcgttgagtaattttggggaaagcttcgtccacagtttttttttcgatgaacagtgccgcagtggcgctgatcttgtatgctatcctgcaatcgtggtgaacttatttcttttatatccttcactcccatgaaaaggctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggcgccttgggatccatggctgcgaccactctcacgagcgctctcccaggagcctttagcagctctcagagaccttcggctccgttcaacctccagaggagccctagagtcctcagacgcttcaaccgcaagaccggtagacagccacgcggtctcgtcagagctgctaaggctcagcgctctggtaccagatccatgggcaactccgttctcaattccggaaggactacgatctgtgatgcgtacaacgttgcagctcatgatccgttctcattccagcacaagtcacttgacactgttcagagggagtggactgagtggaagaagaacaaccattcgctgtatctcgatccgatcgttggaactgtggcttcattcctgctcaagaaggtcggttctctcgttggtaagaggattctctcggaactcaggaacttgatcttcccatctggtagcacaaacctcatgcaggacatacttagggaaactgagcagttcctgaaccaacgccttgacactgataccttggcaagggtcaatgctgagttgacaggtcttcaagcgaacgttgaggagttcaatcgccaagttgacaacttccttaaccctaaccggaatgccgttcctctgtctatcacgtcatctgtcaacacgatgcagcagctgttcttgaaccggcttcctcaattccagatgcaaggttaccaactgttgctccttccactgttcgctcaagctgctaatctgcatctgagcttcatcagggatgtcatcctgaatgccgacgaatggggtatatctgcagctacacttcgcacttacagggactacctgaagaactacacgcgcgactactcgaactactgcatcaacacctatcagtccgccttcaaaggcctgaacacgaggcttcatggtacgttggagtttcggacgtacatgttcctgaacgtgttcgagtatgtctccatctggtcactcttcaagtaccagtcattgctggtctcgtcaggtgctaacctgtacgcatcaggatcaggacctcaacagacgcaatcgttcacgtctcaagactggccattcctgtatagcttgttccaagtcaactccaactacgtgctgaacggcttctctggtgctaggttgtccaacactttcccaaacatcggtggacttccaggaagcactacgactcatgcactgcttgctgcaagggtcaactactctggaggtatctcatctggtgacattggagcttcaccgttcaaccagaacttcaactgcagcacattccttccacctttgcttacgccattcgttagatcatggcttgactctggatctgatagggaaggagtcgctactgtgaccaactggcagacagagtcattcgagacaacactcggtcttcgctcaggagcattcacagcaagaggcaacagcaactacttcccagactacttcattcgcaacatctctggagttcctcttgtcgttaggaacgaggaccttcgcagacctctgcactacaatgagatcaggaacattgcctcaccttcaggtacacctggtggagcaagggcttacatggtctcagttcacaaccgcaagaacaacatccatgcagttcatgagaacggatcgatgatccacttggcacctaacgactacactggattcacgatctcacctatccatgctactcaggtgaacaaccagactcgcactttcatcagcgagaagttcggcaaccaaggcgattctctgaggtttgagcagaacaacacgactgcaaggtacactctcagaggtaacggcaactcgtacaacctgtacttgcgcgtctccagcataggcaactcaacgatccgcgttaccatcaacggtcgcgtttacactgctacaaacgtcaacacgaccactaacaacgatggtgtcaacgacaatggtgctcgcttcagcgacatcaacatcggtaacgttgtcgcaagcagcaactctgacgttcctctggacatcaacgttacgttcaactctggaacacagttcgatttgatgaacaccatgctggttccgacgaacatcagcccattgtactgagttgcgtggaccgaagcttgcgcgcctaggtttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaaagcttgaaaagaaaatgcagccaagcttaccaagtaagttatgtgtattaaccagaggaagagagaatcttgcaaaatttcaacaaacacaaaaagaagtattactacgattggtggagaaagaaaacgattccaaatcttgaactgttgttgtaaaagcatagcagaaagtgggagacaaccgaaatagaaatgactataacttaatttaatgttatcattataatttcttctagcaaatatttagaaagtaaatatcacatcaacctttaatgtaattaagctttctctttttgattcatgtgagatgaaaagaaaaaaaagaagagaaaagtgtagaaaacacatcatttctaagctgaaggtacatagtacccttgtacttttggtttcacctgcatagagaaaacccacaagaatatgacagtctgatttgtcagtctcattctcaagcaacatttctctatccgttactttcatggtgaataacacaatccatcatcaatactttgtgttactcagaaactgaaagttattccgagtcttgcatatctttgggcctactcgtttttctaccattattgctgattgttaagctctcgctacttgaatcggcattgttggagtgggaaggttcaaaaaattggagttatgactagttgtctctttctatgtacgatggagaaaatgaataaacaactgagaaaatggctcttgtttagttgatgatgctcttaagctttccactggttgccatatatgatttgggcatttcactttgatcttaatgggccttgtaaggcccaagactcatgattatctttagttgatgctcttaattaggtgtgggcaaataattcaaactgtatgtacccgaccaaaaccaaagcaaaaataatcgaaccaaaccgaaaatttaaaaataaccgaatgaaaactaaatcctataactgaaagaactgaaaccgaatcaaaatatttaatgtaaccaaaaatatccgaaatataattatattgtcaaaaatattaataatttctagattaaataattaaaaatacttaaaaatttatataaaatagtaaaaatactcgaaaataaccacaaatattcaaaaacaaccgaaatatcccaaaatattcaaagcaaaataaccgaatggataccaaattttaaaaccgaaaaaactggaacaaaaccagaatcgaaccaaaatttcaaaaatcgaataaatactaaactttagaacaaaaaaaaacgataaccgaatgtatacgaaccaaagccgaattagataaccgaacgtccaggactactcttaatctttccgccacttatgatttgggctattactttgtttataatgagccttttcaagctcaagttcatgattgtccgtgagatgagaaactgacttgttggattcgaaaccctagctagtattggttaatacttaatacataaatgacctgcattgacatcatcatccaagaaaataaaaattgtatgcttgagatatttagttttcctagctaggttttctttattttagtaccgaatctttaggtgtgccacgttaatttagacccattttttcatacttaccaactgagtctagtttaatcatgactataatcgtataaaatgattcagtcgacgtcattgcgaacgtatataaaatcatccaaattgacgtcattccaaagaggtaagcatgcttatctaagagtccgagcatactaaacaagacgacattttatttgcactctaaatcaaattttgtattgcctaaagaaaaacaatcaaactcaagtttcttaaaattaatttcattcaaactaatcactttcaatatctcacatattattcatgccatttctatttgtctaaacatgatttaaaaaaaaagtaaaatacaaagattactatgcaaaaactctataaaaaaaaattcaaatttcttatttatttgtgacatcaaattttcaaaataatttttttaattatcggttgatccggtcagtcgataaaaacataaactttcagcgaccgttaaaactttcctactaccgatttagagaaaatcttagcttgaaacgtaattgtaacctgccttcatgcaagtcgcaagatatgtcatcctaagttgtatatgttttctcaaaagatgtatttacttgagaaaatacgtttcaacgttgatggacaaccaattaagaatcaagcacctttcgtaatcaatttaggcttatcgtctaaggtatactgatttacgacagttgactagacttataaggaacaaaataatagaataatttcgtcaagaaaaattgattttggactcatactttacataatattttactcttaaatttatttaagtggctcctcgcatgatcccaaagagcaagcctagactatatggaaaagtttctaaacacttcacctaatcatagagactaagatggtaattcgtaaacgacaaagcctagtgacactgtccattgtaaaattccacatcatattagtattaaacatatacatgtagtttcctgaacacatgtagtatcaaacacacttcgtggcttcttcctcgaaatcgaggcctaggcttaaggtttaaacagcccgggcgcgcccggaccgggccatctaggccccttagggagctctcgcgacgtcaatcgagtacgtacgtaagggcgacaccccctaattagcccgggtctagagtcgacagatctccatggatccgttaacggccactttgtacaagaaagctgggtgcccgggaataagtgactagggtcacgtgaccctagtcacttaggtgaccaagcttcggccgcaggatagaggacatcctggacctactgaacgtcagcaatgacgactgaaagattcccaggacaccggcggaagtggtggacccagtctaggtgcgatgcttagtcgcgcacgatgactatgtcggaaggcatctttgctttcggcaaactttagtaatactttaaggaaagtattgtacaagttaggtgcagagacaataatgcacccagctttagctttgtttatggaattattgtgtcggttgcattattggatgcctgcgtgcaccctaagcaatccccggccctcttctctataagaggagcccttgcaatcagttgcaagcatgcaagtttcccactgcaagcttacttctgagtttgagttcaagttcaataaaattcaagctttcctcttacattctgttcttgaaaggttcgatctaatcgagcgagtagagaacaagatcttttgggatttccgccgttccggatcttcgatatcccggactggcgccaggtccgccttgtttctcctctgtctcttgatctgactaatcttggtttatgattcgttgagtaattttggggaaagcttcgtccacagtttttttttcgatgaacagtgccgcagtggcgctgatcttgtatgctatcctgcaatcgtggtgaacttatttcttttatatccttcactcccatgaaaaggctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggcgccttgggatccatgggccacaacaacccgaacatcaacgagtgcatcccgtacaactgcctgtccaacccggaggtggaggtgcttggaggcgagagaatcgagaccggctacactcccatcgacatcagcctcagccttacccagttcctgctctcggagttcgtgccaggagcaggtttcgtgctgggactggtcgacgtgatctggggcatcttcggtccgtcccaatgggatgcgttcctggttcagatcgagcagctgatcaaccagcgcatcgaggagttcgccaggaaccaggccatctctagggtcgagggcctcagcaacctgtaccagatctacgcagagtccttcagagagtgggaggccgatccgaccaatccagcgctcaaggaggagatgcgcacgcagttcaacgacatgaactccgctctgacgacagccattccgctgtttgcggtccagaactaccaggtgccgctgcttagcgtgtacgtccaggctgctaacctccacctgtcggttcttcgggacgtgtcagtgttcggccagaggtggggattcgacgctgcgacgatcaactcgcgctacaacgacctcaccaggctcatcgggaactacacagaccacgcagtgcgctggcacaacaccgggttggagcggatatggggcccggactcgagagattggattcggtacaaccagttccgccgcgagctgaccctcacggtgctggacatcgtgtcgctgttcccgaactacgactcgcgcacgtacccgatccgcacggcgagccaactgaccagggagatctacaccaacccggttctcgagaacttcgacggcagctttcgcggaagcgcgcaaggcatcgaaggttcgatccgctcgccgcacctgatggacatactcaacagcatcaccatctacacggacgcgcacagaggcgagtactactggagcggacaccagatcatggcgagccctgtcggcttctctggaccagagttcacattcccgctgtacggcacgatgggtaacgctgctccgcaacagaggatcgttgctcagctcggccaaggcgtctacagaaccctgtcctcgactctgtaccggaggccgttcaacatcggcatcaacaaccagcagctttccgtccttgacggtacggagttcgcgtatggcacctcatccaacctgccttccgccgtttaccggaagtccgggacagtggacagcctcgacgagatcccgccgcagaacaacaacgtgcctccaaggcaaggcttctctcacaggctctcacacgtgtcgatgttccgctctgggttcagcaactcctccgtctccatcatccgcgctcccatgttctcgtggattcacaggagcgccgagttcaacaacacgatcgacccggagcgcatcaaccagatcccgctgaccaagagcacgaacctcggctcaggcacctctgtggtcaaaggacccggtttcactggcggcgacatcttgaggaggacaagcccagggcagatctccacgcttcgcgtcaacatcacagctccgctgtcccagcgctaccgcgttcggatcaggtacgcctcgacgaccaacctccaattccacacctcgatcgatgggaggccgatcaaccagggcaacttctccgcgacaatgtcctccggcagcaacttgcagagcggttccttccgcaccgtgggcttcaccacgccgttcaacttcagcaacgggtcctctgtcttcaccctgtcggcacatgtgttcaacagcgggaacgaggtctacatcgaccgcatcgagtttgtgccagccgaggttacgtttgaagcggagtacgacctggagcgcgcgcagaaagtggtcaacgcgctgttcacgtcctcgaaccagatcgggctcaagaccgacgtgacggactaccacatcgaccaggtgtccaacctcgtggactgcctgtccgacgagttctgcctcgacgagaagcgcgaactgtccgagaaggtgaagcacgcgaagcggctgtctgacgagcggaaccttctgcaagacccgaacttcagaggtatcaacaggcaacctgaccgcgggtggcgcggatcgacggacatcacgatccagggcggcgacgacgtgttcaaggagaactacgttacactgcccggcacagtggacgagtgttacccgacctacctgtaccagaagatcgacgagtcgaagctcaaggcgtacacgaggtacgagcttcgcggctacatcgaggactcgcaagacctggagatctacctgatccgctacaacgccaagcacgagatcgtgaacgtgcctggtactggttcactgtggccactgagcgcgcaaagcccgattgggaagtgcggtgaacccaacaggtgcgctcctcacctggaatggaatccggacctggattgttcttgccgcgatggcgagaaatgcgcgcaccactcccaccacttcaccctggacatcgacgtcggttgcaccgatctcaacgaggacttgggcgtgtgggtgatcttcaagatcaagacccaggatgggcacgccaggctcggcaacctggagttcctggaggagaagcctctgcttggtgaagcgcttgccagagtcaagagggcggagaagaagtggcgcgacaagcgcgagaagctccagctggagacgaacatcgtctacaaggaggccaaggagtccgtcgacgccctctttgtgaacagccagtacgaccggctccaggtggacacgaacatcgccatgatccatgcagccgacaagcgggttcacaggatcagggaggcttatcttccggagctgagcgtcataccgggcgtgaacgctgcgatcttcgaggagcttgagggccggatcttcacggcttacagcctctacgacgcgaggaacgtgatcaagaacggcgacttcaacaacggcctgctctgctggaacgtcaagggccacgttgacgtcgaggagcagaacaatcaccggagcgtgctggtgatccctgagtgggaagccgaggtgtctcaggaggtcagggtctgtcctggacgcggatacatccttcgcgtcacagcctacaaggagggctatggcgagggctgcgtcaccattcacgagatcgaggacaacaccgacgagctgaagttcagcaattgcgtcgaggaggaggtgtacccgaacaacaccgtcacctgcaacaactacacgggcacacaggaggagtatgagggcacctacacctctcgcaaccagggctacgatgaagcgtacggcaacaacccatcagttcccgccgactacgcctccgtctacgaggagaagtcgtacaccgacggcagacgcgagaatccttgtgagtccaacagaggctacggcgactacacgccactgccggctggatatgtgaccaaggacctggagtacttcccggagaccgacaaggtgtggatcgagatcggcgagaccgagggaaccttcatcgtcgacagcgtcgagctgctcctgatggaggagtaggttaattcgattactagtgtttttctcagacagttttctaaaaaaagggcgtttctggggaagttcgagatggttcgtaaggtgttactggctcctgtgaaccaatacatgatactgccatgataagggttataattagtcaagcagagtaagaagaaacaacagtagcagtgactccgattcctgaagatgagtcatatttgtcttgtgctcctgctgtatgaaatggatcgcatgtgtatattcgtcgccgcgccgcactggtgtaacctgttgcctcagagtttgcttttagctggttctgttttaaaaataagtactgttttttggttggctgcaagccattctgaacttcagtttaccaattgtttttatgttgtggttgaatattttaattttttatttaatgtttggttctttttttatatatatttgcaaaaatgatacaagtggtcaagttttcatatagtatgggctctatttcctagagctctacctctaggaacgaattttgtggaggttttcttttggctagttaggcaaagtccccatatcttgcaggctaaatcaagaagaagctctgtcaaacagttttttttactgaaaagtgattaaagagtagtttctcctagatcacttcagagtttatcctagagaatcatgggaatcaaattcagttagaggatcatttcttacaaagaatcaactttcgtagagaatctaaagcagaaagagctttgacaaacttacccttagagcaattccaacattctcgcgtgagtttcttcgcgccgttgttttgcggtgacttcatctggacgtcccgcgacatagagacgcttgtattgatcatgagagcttgtgtggtcatacacaatataattgttaaagatgaaagagatgtggaccttaatgagcgattcgactttgatggtgaaaatgtgcaaccttctcatggtatttctactcgcacactagctgaatttattgaagctcataaaaagatccgagacaaagaaatacattttcaattgaaagaagacctaatcaagcacttatgggaattcctaggcttaaggtttaaacagccccctccggcggtgtcccccactgaagaaactatgtgctgtagtatagccgctggctagctagctagttgagtcatttagcggcgatgattgagtaataatgtgtcacgcatcaccatgcatgggtggcagtctcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacgaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtcaacggaagcgctgcagaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagagaattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtgaccaagcttggcgcgccgtcccattctggccgaatttaagtgactagggtcacgtgaccctagtcacttaccggattctggccggagcctgcttttttgtacaaacttgaagctggccttctaggcccggaccgggtgaccaagcttgggccgcgtttaaacttcgaaacgcgtggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatgaacaagaacaacaccaagctgagcacccgcgccctgccgagcttcatcgactacttcaacggcatctacggcttcgccaccggcatcaaggacatcatgaacatgatcttcaagaccgacaccggcggcgacctgaccctggacgagatcctgaagaaccagcagctgctgaacgacatcagcggcaagctggacggcgtgaacggcagcctgaacgacctgatcgcccagggcaacctgaacaccgagctgagcaaggagatccttaagatcgccaacgagcagaaccaggtgctgaacgacgtgaacaacaagctggacgccatcaacaccatgctgcgcgtgtacctgccgaagatcaccagcatgctgagcgacgtgattaagcagaactacgccctgagcctgcagatcgagtacctgagcaagcagctgcaggagatcagcgacaagctggacatcatcaacgtgaacgtcctgatcaacagcaccctgaccgagatcaccccggcctaccagcgcatcaagtacgtgaacgagaagttcgaagagctgaccttcgccaccgagaccagcagcaaggtgaagaaggacggcagcccggccgacatcctggacgagctgaccgagctgaccgagctggcgaagagcgtgaccaagaacgacgtggacggcttcgagttctacctgaacaccttccacgacgtgatggtgggcaacaacctgttcggccgcagcgccctgaagaccgccagcgagctgatcaccaaggagaacgtgaagaccagcggcagcgaggtgggcaacgtgtacaacttcctgatcgtgctgaccgccctgcaggcccaggccttcctgaccctgaccacctgtcgcaagctgctgggcctggccgacatcgactacaccagcatcatgaacgagcacttgaacaaggagaaggaggagttccgcgtgaacatcctgccgaccctgagcaacaccttcagcaacccgaactacgccaaggtgaagggcagcgacgaggacgccaagatgatcgtggaggctaagccgggccacgcgttgatcggcttcgagatcagcaacgacagcatcaccgtgctgaaggtgtacgaggccaagctgaagcagaactaccaggtggacaaggacagcttgagcgaggtgatctacggcgacatggacaagctgctgtgtccggaccagagcgagcaaatctactacaccaacaacatcgtgttcccgaacgagtacgtgatcaccaagatcgacttcaccaagaagatgaagaccctgcgctacgaggtgaccgccaacttctacgacagcagcaccggcgagatcgacctgaacaagaagaaggtggagagcagcgaggccgagtaccgcaccctgagcgcgaacgacgacggcgtctacatgccactgggcgtgatcagcgagaccttcctgaccccgatcaacggctttggcctgcaggccgacgagaacagccgcctgatcaccctgacctgtaagagctacctgcgcgagctgctgctagccaccgacctgagcaacaaggagaccaagctgatcgtgccaccgagcggcttcatcagcaacatcgtggagaacggcagcatcgaggaggacaacctggagccgtggaaggccaacaacaagaacgcctacgtcgaccacaccggcggcgtgaacggcaccaaggccctgtacgtgcacaaggacggcggcatcagccagttcatcggcgacaagctgaagccgaagaccgagtacgtgatccagtacaccgtgaagggcaagccatcgattcacctgaaggacgagaacaccggctacatccactacgaggacaccaacaacaacctggaggactaccagaccatcaacaagcgcttcaccaccggcaccgacctgaagggcgtgtacctgatcctgaagagccagaacggcgacgaggcctggggcgacaacttcatcatcctggagatcagcccgagcgagaagctgctgagcccggagctgatcaacaccaacaactggaccagcaccggcagcaccaacatcagcggcaacaccctgaccctgtaccagggcggcaggggcatcctgaagcagaacctgcagctggacagcttcagcacctaccgcgtgtacttcagcgtgagcggcgacgccaacgtgcgcatccgcaactcccgcgaggtgctgttcgagaagaggtacatgagcggcgccaaggacgtgagcgagatgttcaccaccaagttcgagaaggacaacttctacatcgagctgagccagggcaacaacctgtacggcggcccgatcgtgcacttctacgacgtgagcatcaagtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgacctcgaggcctaggcttaaggtttaaacagcccgggcgcgccggtaccgagctcgaattcggtaacccggtccgggccattctggccgtaccgagctcgaattcggcccaacttttctatacaaagttgatagcgataaatcctgaggatctggtcttcctaaggacccgggatatcggaccgattaaactttaattcggtccgataacttcgtatagcatacattatacgaagttatacctggtggcgccgctagcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcaattcgctagcgaagttcctattccgaagttcctattctctagaaagtataggaacttcagatccaccgggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccctagcgtatacgaagttcctattccgaagttcctattctccagaaagtataggaacttctgtacacctgagctgattccgatgacttcgtaggttcctagctcaagccgctcgtgtccaagcgtcacttacgattagctaatgattacggcatctaggaccgactagctaactaactagtacgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtgaatattctcctcagatctggaaccattccgctccaaaaaatcaaccggacggagccactccgttcccatctcgctctcacagtcacgctccgttccgttcactctacaaccaaacaaaaaacagagccgctctgttccagattaccaaacacaaaatagagcggctccgttcctagaatcatggatggaacggctccgttctacttggctccttaaccaaacactactaatgcatctctgtctctgcaccatatttttttcattactaatgagctctcgtgtccaaatcatctaacaagtgtctatttggttcgtgatcaaatttatcaaaaagattactcatttgctgctagaatttaccaacgtctcacaattgacatgtaaaatacatgccaaacatttttttaatcatagttcgataacttttgcacacagtctttgttatgatatattttagttgtcggctaagcaggccggtaatttattgtaacgcagctgtactgtacatcatgtcattgtcatgtgccgatgtgtccgtgctctggactctggacaggtgggtgcaatagagcgtcggagcctgttgcgaaagcaaacgaaaatggttggactacaaaatctgcggtgaacggactagtttgcctacacatctagaagaactgaagaagaagaagataggtggtgacctgctttgttttcaagggcaagaaaacaaagacattgaggaatggatcgtgggcggcagcatgggtgcacgccacgtgaccccgaattgatgacgggtttggtgaccagataattggagagaatttataagggagggatcatcttgttattcaaaattaaatagtaagaggatttctccaaaccttccctttgataggatcaccaaatcagtcctgagagtttagctgaagtttgagaaagctcaatttgagttaatggtgtaggaaacgggtgacgactaagagggtgggatgaattaggacttctaaaactttcgttaatttaggccacaaataaatccctaaagcaaaatctatgcaaataatcaaactagaatgtgcagactaggttttgtctaagtgttgctatctctaccgcaatgactaagtttcaatctacactgaataagtataaatacaagattgaaacttaaatgcttaatataaatgcggaaacttaaagagcaaagtagagatgcaaactctcgtggatgacgccggtatttttaccgaggtatccggaaccgcgcaaggtcccgactaatcctcgttggtgcccctacgcaaagggaagcctacgcgagggccaagcacctcggtcgagtaactccgtagggagccgcgggccttctccacgcgcaagtggtgctccgctttcggctcctctcggacgctccccgccgtctccactatcgagcttccagctgaaacgccgcgggcctcgttccctccggtacacgatggcggtcgtgacacaaacgcggttgtcacggtctctcaagactcgcgtcccactcggcacatttacaacggctcgcacaagagctaaggggttgtgtggttttacaaaactcactcaactaactagggttcacctagagcaagcgctagagcggtaagcacttcgcaaagcacctatgctaatcaccgagtgattctattaagcacttgggtgtaagagcacttgtgaatgtctattgtatgccttggtatgtctcttgggctcccacactaggaaatggccggttggggtgtatttatagcccccaacacaaaagtagtcgttggaggaaagttgttgctttctgcggtcacaccagacggtccggtggggcaccagacaacgcattgttccttgtccggtgcgcctagccgttaccctgtcagagcaggtgactgttggcgccgcaggcttttcacaccggacagtccggtggtcttccctctgagtgccaccaggaactagttgttggggctgaggttcttggtgcaccagacagtccggcgtgaggacatcggacagtccggtgtgccaccggacagtcccgtgctcctcgcacagacagtccgcaggcaacacgtcttcacttcttggacttctcttagatctttgtcgg

SEQ ID NO: 6 - DNA, 32 bp, Artificial Sequence
  Gel-based detection forward primer
ctcttcaggatgaagagctatgtttaaacgtg

SEQ ID NO: 7 - DNA, 27 bp, Artificial Sequence
  gel-based detection reverse primer
tgattttttggagcggaatggttccag

SEQ ID NO: 8 - protein, 694 aa, Artificial Sequence
  Cry2A.127 variant
MAATTLTSALPGAFSSSQRPSAPFNLQRSPRVLRRFNRKTGRQPRGLVRAAKAQRSGTRSMGNSVLNSGRTTICDAYNVAAHDPFSFQHKSLDTVQREWTEWKKNNHSLYLDPIVGTVASFLLKKVGSLVGKRILSELRNLIFPSGSTNLMQDILRETEQFLNQRLDTDTLARVNAELTGLQANVEEFNRQVDNFLNPNRNAVPLSITSSVNTMQQLFLNRLPQFQMQGYQLLLLPLFAQAANLHLSFIRDVILNADEWGISAATLRTYRDYLKNYTRDYSNYCINTYQSAFKGLNTRLHGTLEFRTYMFLNVFEYVSIWSLFKYQSLLVSSGANLYASGSGPQQTQSFTSQDWPFLYSLFQVNSNYVLNGFSGARLSNTFPNIGGLPGSTTTHALLAARVNYSGGISSGDIGASPFNQNFNCSTFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVASSNSDVPLDINVTFNSGTQFDLMNTMLVPTNISPLY

SEQ ID NO: 9 - protein, 1182 aa, Artificial Sequence
  Cry1A.88 variant
MGHNNPNINECIPYNCLSNPEVEVLGGERIETGYTPIDISLSLTQFLLSEFVPGAGFVLGLVDVIWGIFGPSQWDAFLVQIEQLINQRIEEFARNQAISRVEGLSNLYQIYAESFREWEADPTNPALKEEMRTQFNDMNSALTTAIPLFAVQNYQVPLLSVYVQAANLHLSVLRDVSVFGQRWGFDAATINSRYNDLTRLIGNYTDHAVRWHNTGLERIWGPDSRDWIRYNQFRRELTLTVLDIVSLFPNYDSRTYPIRTASQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNTIDPERINQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKVVNALFTSSNQIGLKTDVTDYHIDQVSNLVDCLSDEFCLDEKRELSEKVKHAKRLSDERNLLQDPNFRGINRQPDRGWRGSTDITIQGGDDVFKENYVTLPGTVDECYPTYLYQKIDESKLKAYTRYELRGYIEDSQDLEIYLIRYNAKHEIVNVPGTGSLWPLSAQSPIGKCGEPNRCAPHLEWNPDLDCSCRDGEKCAHHSHHFTLDIDVGCTDLNEDLGVWVIFKIKTQDGHARLGNLEFLEEKPLLGEALARVKRAEKKWRDKREKLQLETNIVYKEAKESVDALFVNSQYDRLQVDTNIAMIHAADKRVHRIREAYLPELSVIPGVNAAIFEELEGRIFTAYSLYDARNVIKNGDFNNGLLCWNVKGHVDVEEQNNHRSVLVIPEWEAEVSQEVRVCPGRGYILRVTAYKEGYGEGCVTIHEIEDNTDELKFSNCVEEEVYPNNTVTCNNYTGTQEEYEGTYTSRNQGYDEAYGNNPSVPADYASVYEEKSYTDGRRENPCESNRGYGDYTPLPAGYVTKDLEYFPETDKVWIEIGETEGTFIVDSVELLLMEE

SEQ ID NO: 10 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 11 - protein, 183 aa, Streptomyces viridochromogenes
MSPERRPVEIRPATAADMAAVCDIVNHYIETSTVNFRTEPQTPQEWIDDLERLQDRYPWLVAEVEGVVAGIAYAGPWKARNAYDWTVESTVYVSHRHQRLGLGSTLYTHLLKSMEAQGFKSVVAVIGLPNDPSVRLHEALGYTARGTLRAAGYKHGGWHDVGFWQRDFELPAPPRPVRPVTQI

SEQ ID NO: 12 - DNA, 65 bp, Artificial Sequence
  32218 specific amplicon
agttgtctaagcgtcaatttgtgaatattctcctcagatctggaaccattccgctccaaaaaatc

SEQ ID NO: 13 - DNA, 23 bp, Artificial Sequence
  HGM forward primer
ttggactagaaatctcgtgctga

SEQ ID NO: 14 - DNA, 22 bp, Artificial Sequence
  HGM reverse primer
gctacatagggagccttgtcct

SEQ ID NO: 15 - DNA, 15 bp, Artificial Sequence
  HGM probe
gcgtttgtgtggatt

SEQ ID NO: 16 - DNA, 79 bp, Artificial Sequence
  HGM amplicon
ttggactagaaatctcgtgctgattaattgttttacgcgtgcgtttgtgtggattgtaggacaaggctccctatgtagc